(19) United States
(12) Patent Application Publication
(10) Pub. No.: US 2005/0165089 A1
(43) Pub. Date: Jul. 28, 2005

(54) Compounds, compositions and methods

(76) Inventors: Gustave Bergnes, Pacifica, CA (US); Dashyant Dhanak, West Chester, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/959,610

(22) Filed: Oct. 5, 2004

Related U.S. Application Data
(60) Provisional application No. 60/509,400, filed on Oct. 6, 2003.

(51) Int. Cl. A61K 31/353

(52) U.S. Cl. 514/456; 549/403

(57) ABSTRACT

Compounds useful for treating cellular proliferative diseases and disorders by modulating the activity of KSP are disclosed.

### CROSS-REFERENCE TO RELATED APPLICATIONS

This application claims the benefit of U.S. Provisional Patent Application No. 60/509,400, filed Oct. 6, 2003, which is incorporated herein by reference for all purposes.

### FIELD OF THE INVENTION

This invention relates to compounds that are inhibitors of the mitotic kinesin KSP and are useful in the treatment of cellular proliferative diseases, for example cancer, hyperplasias, restenosis, cardiac hypertrophy, immune disorders, fungal disorders, and inflammation.

### BACKGROUND OF THE INVENTION

Among the therapeutic agents used to treat cancer are the taxanes and vinca alkaloids, which act on microtubules. Microtubules are the primary structural element of the mitotic spindle. The mitotic spindle is responsible for distribution of replicate copies of the genome to each of the two daughter cells that result from cell division. It is presumed that disruption of the mitotic spindle by these drugs results in inhibition of cancer cell division, and induction of cancer cell death. However, microtubules form other types of cellular structures, including tracks for intracellular transport in nerve processes. Because these agents do not specifically target mitotic spindles, they have side effects that limit their usefulness.

Improvements in the specificity of agents used to treat cancer is of considerable interest because of the therapeutic benefits which would be realized if the side effects associated with the administration of these agents could be reduced. Traditionally, dramatic improvements in the treatment of cancer are associated with identification of therapeutic agents acting through novel mechanisms. Examples of this include not only the taxanes, but also the camptothecin class of topoisomerase I inhibitors. From both of these perspectives, mitotic kinesins are attractive targets for new anti-cancer agents.

Mitotic kinesins are enzymes essential for assembly and function of the mitotic spindle, but are not generally part of other microtubule structures, such as in nerve processes. Mitotic kinesins play essential roles during all phases of mitosis. These enzymes are “molecular motors” that transform energy released by hydrolysis of ATP into mechanical force which drives the directional movement of cellular cargoes along microtubules. The catalytic domain sufficient for this task is a compact structure of approximately 340 amino acids. During mitosis, kinesins organize microtubules into the bipolar structure that is the mitotic spindle. Kinesins mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death.

Among the mitotic kinesins which have been identified is KSP. KSP belongs to an evolutionarily conserved kinesin subfamily of plus end-directed microtubule motors that assemble into bipolar homotetramers consisting of antiparallel homodimers. During mitosis KSP associates with microtubules of the mitotic spindle. Microinjection of antibodies directed against KSP into human cells prevents spindle pole separation during prometaphase, giving rise to monopolar spindles and causing mitotic arrest and induction of programmed cell death. KSP and related kinesins in other, non-human, organisms, bundle antiparallel microtubules and slide them relative to one another, thus forcing the two spindle poles apart. KSP may also mediate in anaphase B spindle elongation and focussing of microtubules at the spindle pole.

Human KSP (also termed HsEg5) has been described (Blangy, et al., Cell, 83: 1159-69 (1995); Whitehead, et al., Arthritis Rheum., 39: 1635-42 (1996); Galgio et al., J. Cell Biol., 135: 339-414 (1996); Blangy, et al., J. Biol. Chem., 272: 19418-24 (1997); Blangy, et al., Cell Motil Cytoskeleton, 40: 174-82 (1998); Whitehead and Rattner, J. Cell Sci., 111: 2551-61 (1998); Kaiser, et al., JBC 274: 18925-31 (1999); GenBank accession numbers: X85137, NM004523 and U37426), and a fragment of the KSP gene (TRIP5) has been described (Lee, et al., Mol Endocrinol., 9: 243-54 (1995); GenBank accession number L40372). Xenopus KSP homologs (Eg5), as well as Drosophila KLP61 F/KRP1 30 have been reported.

Mitotic kinesins, including KSP, are attractive targets for the discovery and development of novel antimitotic chemotherapeutics. Accordingly, it is an object of the present invention to provide compounds, compositions and methods useful in the inhibition of KSP.

### SUMMARY OF THE INVENTION

In accordance with the objects outlined above, the present invention provides compounds that can be used to treat cellular proliferative diseases. The compounds are KSP inhibitors, such as human KSP inhibitors. The present invention also provides compositions comprising such compounds, and methods utilizing such compounds or compositions, which can be used to treat cellular proliferative diseases.

In one aspect, the invention relates to methods for treating cellular proliferative diseases, and for treating disorders by inhibiting the activity of KSP. The methods employ one or more compounds represented by Formula I: 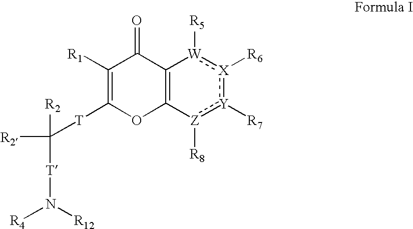
wherein:
W, X, Y, and Z are independently N, C, O, or S, and Z is optionally absent, provided that:
   the ring comprising W, X, Y, and optionally Z is heteroaromatic;
   at least one of W, X, Y, or Z is other than C;
   no more than two of W, X, Y, and Z is —N═; and
   W, X, or Y can be O or S only when Z is absent;
the dashed lines in the structure depict optional double bonds;
T and T′ are independently a covalent bond or optionally substituted lower alkylene;
R₁ is chosen from hydrogen, optionally substituted alkyl-, optionally substituted aryl-, optionally substituted aralkyl-, optionally substituted heteroaryl-, and optionally substituted heteroaralkyl-;
R₂ and R₂, are independently chosen from hydrogen, optionally substituted alkyl-, optionally substituted aryl-, optionally substituted aralkyl-, optionally substituted heteroaryl-, and optionally substituted heteroaralkyl-; or R₂ and R₂′ taken together form an optionally substituted 3- to 7-membered ring which optionally incorporates from one to two additional heteroatoms, selected from N, O, and S, in the ring;
R₁₂ is chosen from hydrogen, optionally substituted alkyl-, optionally substituted aryl-, optionally substituted aralkyl-, optionally substituted heteroaryl-, optionally substituted heteroaralkyl-, —C(O)—R₃, and —S(O)₂—R₃ₐ;
R₄ is chosen from hydrogen, optionally substituted alkyl-, optionally substituted aryl-, optionally substituted aralkyl-, optionally substituted heteroaralkyl-, and optionally substituted heterocyclyl-;
or R₄ taken together with R₁₂, and the nitrogen to which they are bound, form an optionally substituted 5- to 12-membered nitrogen-containing heterocycle, which optionally incorporates from one to two additional heteroatoms, selected from N, O, and S, in the heterocycle ring;
or R₄ taken together with R₂ form an optionally substituted 5- to 12-membered nitrogen-containing heterocycle, which optionally incorporates from one to two additional heteroatoms, selected from N, O, and S, in the heterocycle ring;
R₃ is chosen from hydrogen, optionally substituted alkyl-, optionally substituted aryl-, optionally substituted aralkyl-, optionally substituted heteroaryl-, optionally substituted heteroaralkyl-, R₁₅O—, and R₁₇—NH—;
R₃ₐ is chosen from optionally substituted alkyl-, optionally substituted aryl-, optionally substituted aralkyl-, optionally substituted heteroaryl-, optionally substituted heteroaralkyl-, and R₁₇—NH—;
R₅, R₆, R₇ and R₈ are independently chosen from hydrogen, acyl, optionally substituted alkyl-, optionally substituted alkoxy, halogen, hydroxyl, nitro, cyano, optionally substituted amino, alkylsulfonyl-, alkylsulfonamido-, alkylthio-, carboxyalkyl-, carboxamido-, aminocarbonyl-, optionally substituted aryl, and optionally substituted heteroaryl-, provided that R₅, R₆, R₇ or R₈ is absent where W, X, Y, or Z, respectively, is —N═, O, S, or absent;
R₁₅ is chosen from optionally substituted alkyl-, optionally substituted aryl-, optionally substituted aralkyl-, optionally substituted heteroaryl-, and optionally substituted heteroaralkyl-; and
R₁₇ is hydrogen, optionally substituted alkyl-, optionally substituted aryl-, optionally substituted aralkyl-, optionally substituted heteroaryl-, or optionally substituted hetero-aralkyl-, including single stereoisomers, mixtures of stereoisomers;
a pharmaceutically acceptable salt of a compound of Formula I;
a pharmaceutically acceptable solvate of a compound of Formula I; or a pharmaceutically acceptable solvate of a pharmaceutically acceptable salt of a compound of Formula I.

In some embodiments, the methods employ one or more compounds represented by Formula II: 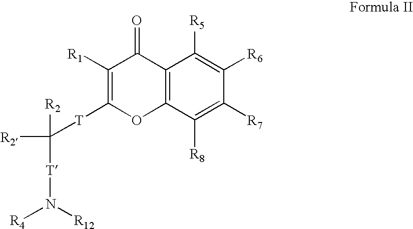
wherein R₁, R₂, R_{2′}, R₄ through R₈, R₁₂, T, and T′ are as defined above; provided that T and T′ are not both covalent bonds, including single stereoisomers, mixtures of stereoisomers;
a pharmaceutically acceptable salt of a compound of Formula II;
a pharmaceutically acceptable solvate of a compound of Formula II; or
a pharmaceutically acceptable solvate of a pharmaceutically acceptable salt of a compound of Formula II.

In some embodiments, the methods employ one or more compounds represented by Formula III: 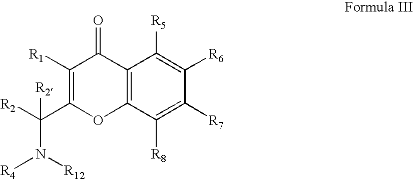
wherein:
R₁ is chosen from hydrogen, optionally substituted alkyl-, optionally substituted aryl-, optionally substituted aralkyl-, optionally substituted heteroaryl-, and optionally substituted heteroaralkyl-;
   R₂ and R₂ are independently chosen from hydrogen, optionally substituted alkyl-, optionally substituted aryl-, optionally substituted aralkyl-, optionally substituted heteroaryl-, and optionally substituted heteroaralkyl-; or R₂ and R₂ taken together form an optionally substituted 3- to 7-membered ring which optionally incorporates from one to two additional heteroatoms, selected from N, O, and S, in the ring;
   R₁₂ taken together with R₄, and the nitrogen to which they are bound, form an optionally substituted 5- to 12-membered nitrogen-containing heterocycle, which optionally incorporates from one to two additional heteroatoms, selected from N, O, and S, in the heterocycle ring, provided that such 5-membered nitrogen-containing heterocycle is not an optionally substituted imidazolyl or imidazolinyl ring; or
   R₄ taken together with R₂ form an optionally substituted 5- to 12-membered nitrogen-containing heterocycle, which optionally incorporates from one to two additional heteroatoms, selected from N, O, and S, in the heterocycle ring; and
   R₁₂ is chosen from hydrogen, optionally substituted alkyl-, optionally substituted aryl-, optionally substituted aralkyl-, optionally substituted heteroaralkyl-, and optionally substituted heterocyclyl-; and
   R₅, R₆, R₇ and R₉ are independently chosen from hydrogen, acyl, optionally substituted alkyl-, optionally substituted alkoxy, halogen, hydroxyl, nitro, cyano, optionally substituted amino, alkylsulfonyl-, alkylsulfonamido-, alkylthio-, carboxyalkyl-, carboxamido-, aminocarbonyl-, optionally substituted aryl, and optionally substituted heteroaryl-; including single stereoisomers, mixtures of stereoisomers;
   a pharmaceutically acceptable salt of a compound of Formula III;
   a pharmaceutically acceptable solvate of a compound of Formula III; or
   a pharmaceutically acceptable solvate of a pharmaceutically acceptable salt of a compound of Formula III.

In another aspect, the invention relates to compounds useful in inhibiting KSP kinesin. The compounds have the structures shown above in Formula I, II, or III; a pharmaceutically acceptable salt of a compound of Formula I, II, or III; a pharmaceutically acceptable solvate of a compound of Formula I, II, or III; or a pharmaceutically acceptable solvate of a pharmaceutically acceptable salt of a compound of Formula I, II, or III. The invention also relates to pharmaceutical compositions containing a therapeutically effective amount of a compound of Formula I, II, or III; a pharmaceutically acceptable salt of a compound of Formula I, II, or III; a pharmaceutically acceptable solvate of a compound of Formula I, II, or III; or a pharmaceutically acceptable solvate of a pharmaceutically acceptable salt of a compound of Formula I, II, or III, admixed with at least one pharmaceutical excipient. In another aspect, the composition further comprises a chemotherapeutic agent other than a compound of the present invention.

In an additional aspect, the present invention provides methods of screening for compounds that will bind to a KSP kinesin, for example compounds that will displace or compete with the binding of a compound of the invention. The methods comprise combining a labeled compound of the invention, a KSP kinesin, and at least one candidate agent and determining the binding of the candidate agent to the KSP kinesin.

In a further aspect, the invention provides methods of screening for modulators of KSP kinesin activity. The methods comprise combining a compound of the invention, a KSP kinesin, and at least one candidate agent and determining the effect of the candidate agent on the KSP kinesin activity.

### DETAILED DESCRIPTION OF THE INVENTION

Definitions

As used in the present specification, the following words and phrases are generally intended to have the meanings as set forth below, except to the extent that the context in which they are used indicates otherwise. The following abbreviations and terms have the indicated meanings throughout:
Ac=acetyl
Boc=t-butyloxy carbonyl
Bu=butyl
c-=cyclo
CBZ=carbobenzoxy=benzyloxycarbonyl
DCM=dichloromethane=methylene chloride=CH₂Cl₂
DIEA=N,N-diisopropylethylamine
DMF=N,N-dimethylformamide
DMSO=dimethyl sulfoxide
Et=ethyl
HBTU=O-benzotriazol-1-yl-N,N,N′,N′-tetramethyluronium hexafluorophosphate
HMDS=hexamethyldisilazane
HOAc=acetic acid
IPA=isopropyl alcohol
Me=methyl
Ph=phenyl
Py=pyridine
rt=room temperature
sat'd=saturated
s-=secondary
t-=tertiary
TEA=triethylamine
TFA=trifluoroacetic acid
THF=tetrahydrofuran
Tf=triflate
XRPD=x-ray powder diffraction

Alkyl is intended to include linear, branched, or cyclic aliphatic hydrocarbon structures and combinations thereof, which structures may be saturated or unsaturated. Lower-alkyl refers to alkyl groups of from 1 to 5 carbon atoms, such as from 1 to 4 carbon atoms. Examples of lower-alkyl groups include methyl, ethyl, propyl, isopropyl, butyl, s-and t-butyl and the like. In some embodiments, alkyl groups are those of C₂₀ or below. In some embodiments, alkyl groups are those of C₁₃ or below. Cycloalkyl is a subset of alkyl and includes cyclic aliphatic hydrocarbon groups of from 3 to 13 carbon atoms. Examples of cycloalkyl groups include c-propyl, c-butyl, c-pentyl, norbornyl, adamantyl and the like. Cycloalkyl-alkyl- is another subset of alkyl and refers to cycloalkyl attached to the parent structure through a non-cyclic alkyl. Examples of cycloalkyl-alkyl- include cyclohexylmethyl, cyclopropylmethyl, cyclohexylpropyl, and the like. In this application, alkyl includes alkanyl, alkenyl and alkynyl residues; it is intended to include vinyl, allyl, isoprenyl and the like. Alkylene-, alkenylene-, and alkynylene- are other subsets of alkyl, including the same residues as alkyl, but having two points of attachment within a chemical structure. Examples of alkylene include ethylene (—CH₂CH₂—), propylene (—CH₂CH₂CH₂—), dimethylpropylene (—CH₂C(CH₃)₂CH₂—) and cyclohexylpropylene (—CH₂CH₂CH(C₆H₁₃)—). Likewise, examples of alkenylene include ethenylene (—CH═CH—), propenylene (—CH═CH—CH₂—), and cyclohexylpropenylene (—CH═CHCH(C₆H₁₃)—). Examples of alkynylene include ethynylene (—C≡C—) and propynylene (—CH≡CH—CH₂—). When an alkyl residue having a specific number of carbons is named, all geometric isomers having that number of carbons are intended to be encompassed; thus, for example, “butyl” is meant to include n-butyl, sec-butyl, isobutyl and t-butyl; “propyl” includes n-propyl, isopropyl, and c-propyl.

Cycloalkenyl is a subset of alkyl and includes unsaturated cyclic hydrocarbon groups of from 3 to 13 carbon atoms. Examples of cycloalkenyl groups include c-hexenyl-, c-pentenyl and the like.

Alkoxy or alkoxyl refers to an alkyl group, such as including from 1 to 8 carbon atoms, of a straight, branched, or cyclic configuration, or a combination thereof, attached to the parent structure through an oxygen (i.e., the group alkyl-O—). Examples include methoxy-, ethoxy-, propoxy-, isopropoxy-, cyclopropyloxy-, cyclohexyloxy- and the like. Lower-alkoxy refers to alkoxy groups containing one to four carbons.

Acyl refers to groups of from 1 to 8 carbon atoms of a straight, branched, or cyclic configuration or a combination thereof, attached to the parent structure through a carbonyl functionality. Such groups may be saturated or unsaturated, and aliphatic or aromatic. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to acyl groups containing one to four carbons.

Amino refers to the group —NH₂. The term “substituted amino” refers to the group —NHR or —NRR where each R is independently selected from the group: optionally substituted alkyl, optionally substituted alkoxy, optionally substituted amino carbonyl, optionally substituted aryl, optionally substituted heteroaryl, optionally substituted heterocyclyl, acyl, alkoxycarbonyl, sulfinyl and sulfonyl, e.g., diethylamino, methylsulfonylamino, furanyl-oxy-sulfonamino.

Aminocarbonyl—refers to the group —NR^{c}COR^{b}, —NR^{c}CO₂R^{a}, or —NR^{c}CONR^{b}R^{c}, where
R^{a} is optionally substituted C₁-C₆ alkyl, aryl, heteroaryl, aryl-C₁-C₄ alkyl-, or heteroaryl-C₁-C₄ alkyl-group;
R^{b} is H or optionally substituted C₁-C₆ alkyl, aryl, heteroaryl, aryl-C₁-C₄ alkyl-, or heteroaryl-C₁-C₄ alkyl-group; and
R^{c} is hydrogen or C₁-C₄ alkyl; and
   where each optionally substituted R^{b} group is independently unsubstituted or substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

Antimitotic refers to a drug for inhibiting or preventing mitosis, for example, by causing metaphase arrest. Some antitumour drugs block proliferation and are considered antimitotics.

Aryl and heteroaryl mean a 5- or 6-membered aromatic or heteroaromatic ring containing 0 or 1-4 heteroatoms, respectively, selected from O, N, or S; a bicyclic 9- or 10-membered aromatic or heteroaromatic ring system containing 0 or 1-4 (or more) heteroatoms, respectively, selected from O, N, or S; or a tricyclic 12- to 14-membered aromatic or heteroaromatic ring system containing 0 or 1-4 (or more) heteroatoms, respectively, selected from O, N, or S. The aromatic 6- to 14-membered carbocyclic rings include, e.g., phenyl, naphthyl, indanyl, tetralinyl, and fluorenyl and the 5- to 10-membered aromatic heterocyclic rings include, e.g., imidazolyl, pyridinyl, indolyl, thienyl, benzopyranonyl, thiazolyl, furanyl, benzimidazolyl, quinolinyl, isoquinolinyl, quinoxalinyl, pyrimidinyl, pyrazinyl, tetrazolyl and pyrazolyl.

Aralkyl—refers to a residue in which an aryl moiety is attached to the parent structure via an alkyl residue. Examples include benzyl, phenethyl, phenylvinyl, phenylallyl and the like. Heteroaralkyl—refers to a residue in which a heteroaryl moiety is attached to the parent structure via an alkyl residue. Examples include furanylmethyl, pyridinylmethyl, pyrimidinylethyl and the like.

Aralkoxy—refers to the group —O-aralkyl. Similarly, heteroaralkoxy—refers to the group —O-heteroaralkyl; aryloxy—refers to the group —O-aryl; acyloxy—refers to the group —O-acyl; heteroaryloxy—refers to the group —O-heteroaryl; and heterocyclyloxy—refers to the group —O-heterocyclyl (i.e., aralkyl, heteroaralkyl, aryl, acyl, heterocyclyl, or heteroaryl is attached to the parent structure through an oxygen).

Carboxyalkyl—refers to the group —alkyl-COOH.

Carboxamido refers to the group —CONR^{b}R^{c}, where
R^{b} is H or optionally substituted C₁-C₆ alkyl, aryl, heteroaryl, aryl-C₁-C₄ alkyl-, or heteroaryl-C₁-C₄ alkyl-group; and
R^{c} is hydrogen or C₁-C₄ alkyl; and
where each optionally substituted R^{b} group is independently unsubstituted or substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

Halogen or halo refers to fluorine (or fluoro), chlorine (or chloro), bromine (or bromo) or iodine (or iodo). Fluorine, chlorine and bromine are preferred. Dihaloaryl, dihaloalkyl, trihaloaryl etc. refer to aryl and alkyl substituted with the designated plurality of halogens (here, 2, 2 and 3, respectively), but not necessarily a plurality of the same halogen; thus 4-chloro-3-fluorophenyl is within the scope of dihaloaryl.

Heterocyclyl means a cycloalkyl or aryl residue in which one to four of the carbons is replaced by a heteroatom such as oxygen, nitrogen or sulfur. Examples of heterocycles that fall within the scope of the invention include azetidinyl, imidazolinyl, pyrrolidinyl, pyrazolyl, pyrrolyl, indolyl, quinolinyl, isoquinolinyl, tetrahydroisoquinolinyl, benzofuranyl, benzodioxanyl, benzodioxyl (commonly referred to as methylenedioxyphenyl, when occurring as a substituent), tetrazolyl, morpholinyl, thiazolyl, pyridinyl, pyridazinyl, piperidinyl, pyrimidinyl, thienyl, furanyl, oxazolyl, oxazolinyl, isoxazolyl, dioxanyl, tetrahydrofuranyl and the like. “N-heterocyclyl” refers to a nitrogen-containing heterocycle. The term heterocyclyl encompasses heteroaryl, which is a subset of heterocyclyl. Examples of N-heterocyclyl residues include azetidinyl, 4-morpholinyl, 4-thiomorpholinyl, 1-piperidinyl, 1-pyrrolidinyl, 3-thiazolidinyl, piperazinyl and 4-(3,4-dihydrobenzoxazinyl). Examples of substituted heterocyclyl include 4-methyl-1-piperazinyl and 4-benzyl-1-piperidinyl.

A leaving group or atom is any group or atom that will, under the reaction conditions, cleave from the starting material, thus promoting reaction at a specified site. Suitable examples of such groups unless otherwise specified are halogen atoms, mesyloxy, p-nitrobenzensulphonyloxy and tosyloxy groups.

Optional or optionally means that the subsequently described event or circumstance may or may not occur, and that the description includes instances where said event or circumstances occurs and instances in which it does not. For example, “optionally substituted alkyl” includes “alkyl” and “substituted alkyl” as defined herein. It will be understood by those skilled in the art with respect to any group containing one or more substituents that such groups are not intended to introduce any substitution or substitution patterns that are sterically impractical and/or synthetically non-feasible and/or inherently unstable.

Substituted alkoxy refers to alkoxy wherein the alkyl constituent is substituted (i.e., —O-(substituted alkyl)). In some embodiments, a substituted alkoxy group is “polyalkoxy” or —O-(optionally substituted alkylene)— (optionally substituted alkoxy), and includes groups such as —OCH₂CH₂OCH₃, and residues of glycol ethers such as polyethyleneglycol, and —O(CH₂CH₂O)ₓCH₃, where x is an integer of about 2-20, such as about 2-10, and for example, about 2-5. Another substituted alkoxy group is hydroxyalkoxy or —OCH₂(CH₂)_{y}OH, where y is an integer of about 1-10, such as about 1-4.

Substituted—alkyl, aryl, and heteroaryl, which includes the substituted alkyl, aryl and heteroaryl moieties of any group containing an optionally substituted alkyl, aryl and heteroaryl moiety (e.g., alkoxy, aralkyl and heteroaralkyl), refer respectively to alkyl, aryl, and heteroaryl wherein one or more (up to about 5, such as up to about 3) hydrogen atoms are replaced by a substituent independently selected from the group:
—R^{a}, —OR^{b}, —O(C₁-C₂ alkyl)O— (e.g., methylenedioxy-), —SR^{b}, guanidine, guanidine wherein one or more of the guanidine hydrogens are replaced with a lower-alkyl group, —NR^{b}R^{c}, halogen, cyano, nitro, —COR^{b}, —CO₂R^{b}, —CONR^{b}R^{c}, —OCOR^{b}, —OCO₂R^{a}, —OCONR^{b}R^{c}, —NR^{c}COR^{b}, —NR^{c}CO₂R^{a}, —NR^{c}CONR^{b}R^{c}, —CO₂R^{b}, —CONR^{b}R^{c}, —NR^{c}OR^{b}, —SOR^{a}, —SO₂R^{a}, —SO₂NR^{b}R^{c}, and —NR^{c}SO₂R^{a},
where R^{a} is an optionally substituted C₁-C₆ alkyl, aryl, heteroaryl, aryl-C₁-C₄ alkyl-, or heteroaryl-C₁-C₄ alkyl-group,
R^{b} is H or optionally substituted C₁-C₆ alkyl, aryl, heteroaryl, aryl-C₁-C₄ alkyl-, or heteroaryl-C₁-C₄ alkyl-group;
R^{c} is hydrogen or C₁-C₄ alkyl;
   where each optionally substituted R^{a} group and R^{b} group is independently unsubstituted or substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl). In the compounds of Formula I or II where T and/or T′ are substituted alkylene, the term “substituted” also refers to alkylene groups where one or more (up to about 3, such as 1) carbon atoms are replaced by a heteroatom independently selected from O, N or S, such as —CH₂—S—CH₂—.

Sulfanyl refers to the groups: —S-(optionally substituted alkyl), —S-(optionally substituted aryl), —S-(optionally substituted heteroaryl), and —S-(optionally substituted heterocyclyl).

Sulfinyl refers to the groups: —S(O)—H, —S(O)— (optionally substituted alkyl), —S(O)— optionally substituted aryl), —S(O)— (optionally substituted heteroaryl), —S(O)— (optionally substituted heterocyclyl); and —S(O)— (optionally substituted amino).

Sulfonyl refers to the groups: —S(O₂)—H, —S(O₂)— (optionally substituted alkyl), —S(O₂)— optionally substituted aryl), —S(O₂)— (optionally substituted heteroaryl), —S(O₂)— (optionally substituted heterocyclyl), —S(O₂)— (optionally substituted alkoxy), —S(O₂)— optionally substituted aryloxy), —S(O₂)— (optionally substituted heteroaryloxy), —S(O₂)— (optionally substituted heterocyclyloxy); and —S(O₂)— (optionally substituted amino).

Pharmaceutically acceptable salts refers to those salts that retain the biological effectiveness of the free compound and that are not biologically or otherwise undesirable, formed with a suitable acid or base, and includes pharmaceutically acceptable acid addition salts and base addition salts. Pharmaceutically acceptable acid addition salts include those derived from inorganic acids such as hydrochloric acid, hydrobromic acid, sulfuric acid, nitric acid, phosphoric acid and the like, and those derived from organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

Pharmaceutically acceptable base addition salts include those derived from inorganic bases such as sodium, potassium, lithium, ammonium, calcium, magnesium, iron, zinc, copper, manganese, aluminum salts and the like. In some embodiments, the pharmaceutically acceptable base addition salt is chosen from ammonium, potassium, sodium, calcium, and magnesium salts. Base additioin salts also include those derived from pharmaceutically acceptable organic non-toxic bases, including salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

Protecting group has the meaning conventionally associated with it in organic synthesis, i.e. a group that selectively blocks one or more reactive sites in a multifunctional compound such that a chemical reaction can be carried out selectively on another unprotected reactive site and such that the group can readily be removed after the selective reaction is complete. A variety of protecting groups are disclosed, for example, in T. H. Greene and P. G. M. Wuts, Protective Groups in Organic Synthesis, Third Edition, John Wiley & Sons, New York (1999), which is incorporated herein by reference in its entirety. For example, a hydroxy protected form is where at least one of the hydroxyl groups present in a compound is protected with a hydroxy protecting group. Likewise, amines and other reactive groups may similarly be protected.

Solvate refers to the compound formed by the interaction of a solvent and a compound of Formula I, II, or III or salt thereof. Suitable solvates of the compounds of the Formula I, II, or III are pharmaceutically acceptable solvates, such as hydrates, including monohydrates and hemi-hydrates.

Many of the compounds described herein contain one or more asymmetric centers (e.g. the carbon to which R₂ and R_{2′} are attached where R₂ differs from R_{2′}) and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms and rotational isomers are also intended to be included.

When desired, the R- and S-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where the desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents, or by converting one enantiomer to the other by asymmetric transformation.

Compounds of the Present Invention

The present invention is directed to a class of novel compounds that are inhibitors of one or more mitotic kinesins. By inhibiting mitotic kinesins, but not other kinesins (e.g., transport kinesins), specific inhibition of cellular proliferation is accomplished. While not intending to be bound by any theory, the present invention capitalizes on the finding that perturbation of mitotic kinesin function causes malformation or dysfunction of mitotic spindles, frequently resulting in cell cycle arrest and cell death. According to some embodiments of the invention, the compounds described herein inhibit the mitotic kinesin, KSP. In some embodiments, the compounds inhibit the mitotic kinesin, KSP, as well as modulating one or more of the human mitotic kinesins selected from the group consisting of HSET (see, U.S. Pat. No. 6,361,993, which is incorporated herein by reference); MCAK (see, U.S. Pat. No. 6,331,424, which is incorporated herein by reference); CENP-E (see, U.S. Pat. No. 6,645,748, which is incorporated herein by reference); Kif4 (see, U.S. Pat. No. 6,440,684, which is incorporated herein by reference); MKLP1 (see, U.S. Pat. No. 6,448,025, which is incorporated herein by reference); Kif15 (see, U.S. Pat. No. 6,355,466, which is incorporated herein by reference); Kid (see, U.S. Pat. No. 6,387,644, which is incorporated herein by reference); Mpp1, CMKrp, KinI-3 (see, U.S. Pat. No. 6,461,855, which is incorporated herein by reference); Kip3a (see, U.S. Pat. No. 6,680,369, which is incorporated herein by reference); Kip3d (see, U.S. Pat. No. 6,492,151, which is incorporated herein by reference); and RabK6.

The methods of inhibiting a human KSP kinesin comprise contacting an inhibitor of the invention with a kinesin, such a human kinesin, such as human KSP or fragments and variants thereof. The inhibition can be of the ATP hydrolysis activity of the KSP kinesin and/or the mitotic spindle formation activity, such that the mitotic spindles are disrupted. Meiotic spindles may also be disrupted.

An object of the present invention is to develop inhibitors of mitotic kinesins, in particular KSP, such as human KSP, for the treatment of disorders associated with cell proliferation. Traditionally, dramatic improvements in the treatment of cancer, one type of cellular proliferative disorder, have been associated with identification of therapeutic agents acting through novel mechanisms. Examples of this include not only the taxane class of agents that appear to act on microtubule formation, but also the camptothecin class of topoisomerase I inhibitors. The compounds, compositions and methods described herein can differ in their selectivity and are used to treat diseases of cellular proliferation, including, but not limited to cancer, hyperplasias, restenosis, cardiac hypertrophy, immune disorders, fungal disorders and inflammation.

Accordingly, the present invention relates to methods employing compounds represented by Formula I, II, or III: 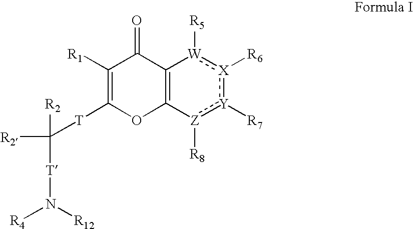
wherein:
W, X, Y, and Z are independently N, C, O, or S, and Z is optionally absent, provided that:
   the ring comprising W, X, Y, and optionally Z is heteroaromatic;
   at least one of W, X, Y, or Z is other than C;
   no more than two of W, X, Y, and Z is —N═; and
   W, X, or Y can be O or S only when Z is absent;
the dashed lines in the structure depict optional double bonds;
T and T′ are independently a covalent bond or optionally substituted lower alkylene;
R₁ is chosen from hydrogen, optionally substituted alkyl-, optionally substituted aryl-, optionally substituted aralkyl-, optionally substituted heteroaryl-, and optionally substituted heteroaralkyl-;
R₂ and R_{2′} are independently chosen from hydrogen, optionally substituted alkyl-, optionally substituted aryl-, optionally substituted aralkyl-, optionally substituted heteroaryl-, and optionally substituted heteroaralkyl-; or R₂ and R₂ taken together form an optionally substituted 3- to 7-membered ring which optionally incorporates from one to two additional heteroatoms, selected from N, O, and S, in the ring;
R₁₂ is chosen from hydrogen, optionally substituted alkyl-, optionally substituted aryl-, optionally substituted aralkyl-, optionally substituted heteroaryl-, optionally substituted heteroaralkyl-, —C(O)—R₃, and —S(O)₂—R₃ₐ;
R₄ is chosen from hydrogen, optionally substituted alkyl-, optionally substituted aryl-, optionally substituted aralkyl-, optionally substituted heteroaralkyl-, and optionally substituted heterocyclyl-;
or R₄ taken together with R₁₂, and the nitrogen to which they are bound, form an optionally substituted 5- to 12-membered nitrogen-containing heterocycle, which optionally incorporates from one to two additional heteroatoms, selected from N, O, and S, in the heterocycle ring;
or R₄ taken together with R₂ form an optionally substituted 5- to 12-membered nitrogen-containing heterocycle, which optionally incorporates from one to two additional heteroatoms, selected from N, O, and S, in the heterocycle ring;
R₃ is chosen from hydrogen, optionally substituted alkyl-, optionally substituted aryl-, optionally substituted aralkyl-, optionally substituted heteroaryl-, optionally substituted heteroaralkyl-, R₁₅O—, and R₁₇—NH—;
R₃ₐ is chosen from optionally substituted alkyl-, optionally substituted aryl-, optionally substituted aralkyl-, optionally substituted heteroaryl-, optionally substituted heteroaralkyl-, and R₁₇—NH—;
R₅, R₆, R₇ and R₈ are independently chosen from hydrogen, acyl, optionally substituted alkyl-, optionally substituted alkoxy, halogen, hydroxyl, nitro, cyano, optionally substituted amino, alkylsulfonyl-, alkylsulfonamido-, alkylthio-, carboxyalkyl-, carboxamido-, aminocarbonyl-, optionally substituted aryl, and optionally substituted heteroaryl-, provided that R₅, R₆, R₇ or R₈ is absent where W, X, Y, or Z, respectively, is —N═, O, S, or absent;
R₁₅ is chosen from optionally substituted alkyl-, optionally substituted aryl-, optionally substituted aralkyl-, optionally substituted heteroaryl-, and optionally substituted heteroaralkyl-; and
R₁₇ is hydrogen, optionally substituted alkyl-, optionally substituted aryl-, optionally substituted aralkyl-, optionally substituted heteroaryl-, or optionally substituted hetero-aralkyl-, including single stereoisomers, mixtures of stereoisomers;
a pharmaceutically acceptable salt of a compound of Formula I;
a pharmaceutically acceptable solvate of a compound of Formula I; or
a pharmaceutically acceptable solvate of a pharmaceutically acceptable salt of a compound of Formula I.

In some embodiments, the methods employ compounds represented by Formula II: 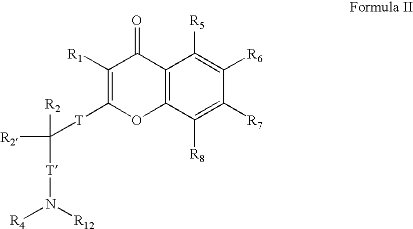
wherein R₁, R₂, R_{2′}, R₄ through R₈, R₁₂, T, and T′ are as defined above; provided that T and T′ are not both covalent bonds, including single stereoisomers, mixtures of stereoisomers;
a pharmaceutically acceptable salt of a compound of Formula II;
a pharmaceutically acceptable solvate of a compound of Formula II; or
a pharmaceutically acceptable solvate of a pharmaceutically acceptable salt of a compound of Formula II.

In some embodiments, the methods employ one or more compounds represented by Formula III: 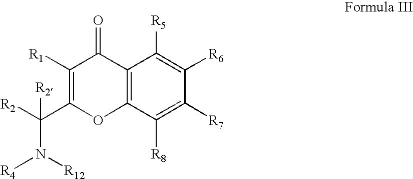
wherein:
R₁ is chosen from hydrogen, optionally substituted alkyl-, optionally substituted aryl-, optionally substituted aralkyl-, optionally substituted heteroaryl-, and optionally substituted heteroaralkyl-;
R₂ and R₂ are independently chosen from hydrogen, optionally substituted alkyl-, optionally substituted aryl-, optionally substituted aralkyl-, optionally substituted heteroaryl-, and optionally substituted heteroaralkyl-; or R₂ and R₂ taken together form an optionally substituted 3- to 7-membered ring which optionally incorporates from one to two additional heteroatoms, selected from N, O, and S, in the ring;
R₁₂ taken together with R₄, and the nitrogen to which they are bound, form an optionally substituted 5- to 12-membered nitrogen-containing heterocycle, which optionally incorporates from one to two additional heteroatoms, selected from N, O, and S, in the heterocycle ring, provided that such 5-membered nitrogen-containing heterocycle is not an optionally substituted imidazolyl or imidazolinyl ring; or
R₄ taken together with R₂ form an optionally substituted 5- to 12-membered nitrogen—containing heterocycle, which optionally incorporates from one to two additional heteroatoms, selected from N, O, and S, in the heterocycle ring; and
R₁₂ is chosen from hydrogen, optionally substituted alkyl-, optionally substituted aryl-, optionally substituted aralkyl-, optionally substituted heteroaralkyl-, and optionally substituted heterocyclyl-; and
R₅, R₆, R₇ and R₉ are independently chosen from hydrogen, acyl, optionally substituted alkyl-, optionally substituted alkoxy, halogen, hydroxyl, nitro, cyano, optionally substituted amino, alkylsulfonyl-, alkylsulfonamido-, alkylthio-, carboxyalkyl-, carboxamido-, aminocarbonyl-, optionally substituted aryl, and optionally substituted heteroaryl-; including single stereoisomers, mixtures of stereoisomers;
a pharmaceutically acceptable salt of a compound of Formula III;
a pharmaceutically acceptable solvate of a compound of Formula III; or
a pharmaceutically acceptable solvate of a pharmaceutically acceptable salt of a compound of Formula III.

In some embodiments, R₂ differs from R₂ and the stereogenic center to which R₂ and R₂ are attached is of the R configuration.

Nomenclature

The compounds of Formula I, II, or III can be named and numbered in the manner (e.g., using AutoNom version 2.1 or ISIS-DRAW, each of which utilizes the IUPAC system of nomenclature) described below. For example, the compound: 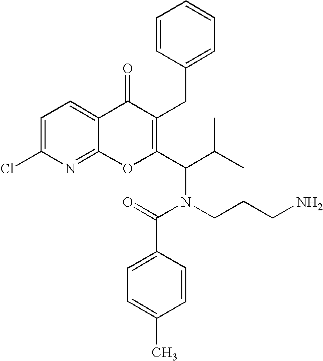
i.e., the compound according to Formula I where W; X, and Y are C, and Z is N, T and T′ are absent (i.e., covalent bonds), R₁ is benzyl, R₂ is i-propyl; R₂ is hydrogen; R₁₂ is —(CO)(R₃); R₃ is 4-methylphenyl-; R₄ is 3-aminopropyl-; R₅ and R₆ are hydrogen, R₇ is chloro; and R₈ is absent is named N-(3-aminopropyl)-N-{1-[7-chloro-4-oxo-3-(phenylmethyl)-4H-pyrano[2,3-b]pyridin-2-yl]-2-methylpropyl}-4-methylbenzamide.

Likewise, the compound: 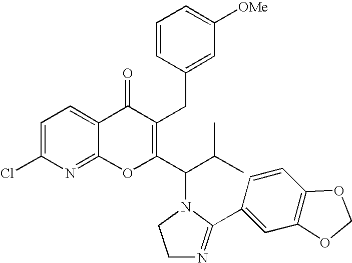
i.e., the compound according to Formula I where W, X and Y are C and Z is N, T and T′ are absent (i.e., covalent bonds), R₁ is 3-methoxy-benzyl, R₂ is i-propyl, R_{2′} is hydrogen, R₄ and R₁₂ taken together form a substituted imidazoline; R₅ and R₆ are hydrogen, R₇ is chloro and R₈ is absent can be named 2-{1-[2-(1,3-benzodioxol-5-yl)-4,5-dihydro-1H-imidazol-1-yl]-2-methylpropyl}-7-chloro-3-{[3-(methyloxy)phenyl]methyl}-4H-pyrano[2,3-b]pyridin-4-one.

Likewise, the compound: 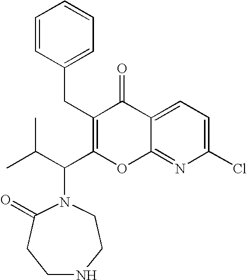
i.e., the compound according to Formula I where W, X and Y are C and Z is N, T and T′ are absent (i.e., covalent bonds), R₁ is benzyl, R₂ is i-propyl, R₂ is hydrogen, R₄ and R₁₂ taken together form a substituted diazepinone ring, R₅ and R₆ are hydrogen, R₇ is chloro, and R₈ is absent can be named 7-chloro-2-[2-methyl-1-(7-oxohexahydro-1H-1,4-diazepin-1-yl)propyl]-3-(phenylmethyl)-4H-pyrano[2,3-b]pyridin-4-one.

### Synthesis of the Compounds of Formula I, II, and III

The compounds of Formula I, II, and III can be prepared by following the procedures described with reference to the Reaction Schemes below or utilizing techniques well known in the art. See, for example, Hirao et al. (1984) Synthesis 1076-1078; Cecchi et al. (1988) Journal of Heterocyclic Chemistry 1367-1371 and Coppola et al. (1981) Synthesis 523-526, which are incorporated herein by reference.

Unless specified otherwise, the terms “solvent”, “inert organic solvent” or “inert solvent” mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary, the solvents used in the reactions of the present invention are inert organic solvents.

In general, esters of carboxylic acids may be prepared by conventional esterification procedures, for example alkyl esters may be prepared by treating the required carboxylic acid with the appropriate alkanol, generally under acidic conditions. Likewise, amides may be prepared using conventional amidation procedures, for example amides may be prepared by treating the relevant activated carboxylic acid with the appropriate amine. Alternatively, a lower-alkyl ester such as a methyl ester of the acid may be treated with an amine to provide the required amide, optionally in presence of trimethylalluminium following the procedure described in Tetrahedron Lett. 48, 4171-4173, (1977). Carboxyl groups may be protected as alkyl esters, for example methyl esters, which esters may be prepared and removed using conventional procedures, one convenient method for converting carbomethoxy to carboxyl is to use aqueous lithium hydroxide.

The salts and solvates of the compounds mentioned herein may as required be produced by methods conventional in the art. For example, if an inventive compound is an acid, a desired base addition salt can be prepared by treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine and arginine; ammonia; primary, secondary, and tertiary amines; such as ethylenediamine, and cyclic amines, such as cyclohexylamine, piperidine, morpholine, and piperazine; as well as inorganic salts derived from sodium, calcium, potassium, magnesium, manganese, iron, copper, zinc, aluminum, and lithium.

If a compound is a base, a desired acid addition salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid, or the like.

Isolation and purification of the compounds and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples hereinbelow. However, other equivalent separation or isolation procedures can, of course, also be used.

### Brief Description Of Reaction Schemes

Reaction Scheme 1 illustrates a synthesis of compounds of formula 109, which can be used as intermediates for the synthesis of other compounds of Formula I, II, or III

Reaction Scheme 2 illustrates a synthesis of compounds of Formula I wherein R₁₂ is —C(O)R₃. One of skill in the art will appreciate that it can be readily applied to compounds of Formula II.

Reaction Scheme 3 illustrates a synthesis of compounds of Formula I wherein R₁₂ is —S(O)₂R₃ₐ. One of skill in the art will appreciate that it can be readily applied to compounds of Formula II.

Reaction Scheme 4 illustrates a synthesis of compounds of Formula I. One of skill in the art will appreciate that it can be readily applied to compounds of Formula II.

Reaction Scheme 5 illustrates a synthesis of compounds of Formula I wherein R₁₂ together with R₄ and the nitrogen to which they are bound, form an optionally substituted imidazolyl. One of skill in the art will appreciate that it can be readily applied to compounds of Formula II.

Reaction Scheme 6 illustrates another synthesis of compounds of Formula I wherein R₁₂ together with R₄ and the nitrogen to which they are bound, form an optionally substituted imidazolyl. One of skill in the art will appreciate that it can be readily applied to compounds of Formula II.

Reaction Scheme 7 illustrates a synthesis of compounds of Formula I wherein R₁₂ together with R₄ and the nitrogen to which they are bound, form an optionally substituted imidazolinyl. One of skill in the art will appreciate that it can be readily applied to compounds of Formula II.

Reaction Scheme 8 illustrates a second synthesis of compounds of Formula I wherein R₁₂ together with R₄ and the nitrogen to which they are bound, form an optionally substituted imidazolinyl. One of skill in the art will appreciate that it can be readily applied to compounds of Formula II.

Reaction Scheme 9 illustrates a synthesis of compounds of Formula I wherein R₁₂ is —C(O)R₃ wherein R₃ is —OR₁₅. One of skill in the art will appreciate that it can be readily applied to compounds of Formula II.

Reaction Scheme 10 illustrates a synthesis of compounds of Formula I wherein R₁₂ is —C(O)R₃ wherein R₃ is —NHR₁₇. One of skill in the art will appreciate that it can be readily applied to compounds of Formula II.

Reaction Scheme 11 illustrates a synthesis of compounds of Formula 1107, which can be used as intermediates in the synthesis of compounds of Formula I, II, or III.

Reaction Scheme 12 illustrates a synthesis of compounds of Formula 105, which can be used as intermediates in the synthesis of compounds of Formula I, II, or III.

Reaction Scheme 13 illustrates a synthesis of compounds of Formula I wherein R₁₂ together with R₄ and the nitrogen to which they are bound, form an optionally substituted diazepinone. One of skill in the art will appreciate that it can be readily applied to compounds of Formula II or III.

Reaction Scheme 14 illustrates a synthesis of compounds of Formula I wherein R₁₂ together with R₄ and the nitrogen to which they are bound, form an optionally substituted diazepinone. One of skill in the art will appreciate that it can be readily applied to compounds of Formula II or III.

Reaction Scheme 15 illustrates a synthesis of compounds of Formula I wherein R₁₂ together with R₄ and the nitrogen to which they are bound, form an optionally substituted piperazine ring. One of skill in the art will appreciate that it can be readily applied to compounds of Formula II or III.

Reaction Scheme 16 illustrates a synthesis of compounds of Formula I wherein wherein R₄ taken together with R₂ form an optionally substituted 5- to 12-membered nitrogen-containing heterocycle.

Starting Materials

The optionally substituted compounds of Formula 101 and other reactants are commercially available, e.g., from Aldrich Chemical Company, Milwaukee, Wis., or may be readily prepared by those skilled in the art using commonly employed synthetic methodology. See, for example, PCT WO 03/39460, WO 03/49678, WO 03/50122, WO 03/49527, WO 03/49679, WO 03/50064, and PCT/US03/11432, each of which is incorporated herein by reference for all purposes. 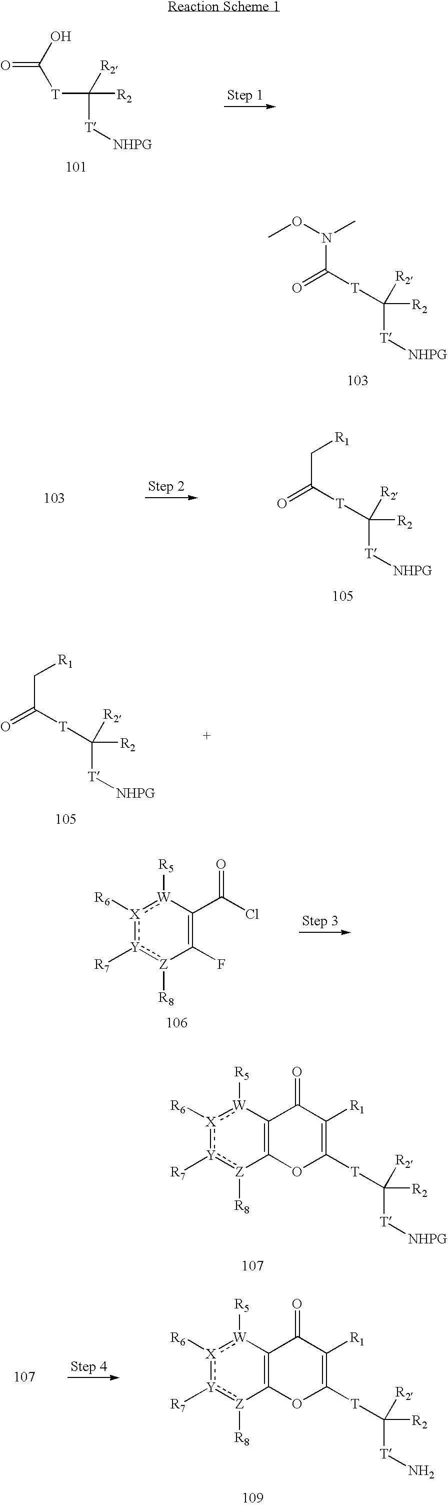
Preparation of Compounds of Formula 103

Referring to Reaction Scheme 1, Step 1, about an equivalent of ethyl chloroformate is added over about one minute to a 0-5° C. solution of a compound of Formula 101 (such as wherein the amino protecting group, PG, is a Boc group) and a base such as triethylamine in a nonpolar, aprotic solvent such as THF. After about 15 minutes, a mixture of an excess of dimethylhydroxylamine hydrochloride (such as about 1.2 equivalents) and a base such as triethylamine in a nonpolar, aprotic solvent such as THF is added over about 5 minutes. The product, a compound of Formula 103, is isolated and used without further purification.

Preparation of Compounds of Formula 105

Referring to Reaction Scheme 1, Step 2, a Grignard reagent is prepared by mixing a compound of formula R₁CH₂Br (generally about 3 equivalents) and magnesium turnings in a nonpolar, aprotic solvent such as diethyl ether. After about 1.5 hours, the Grignard reaction is generally complete. A solution of a compound of Formula 103 in a nonpolar, aprotic solvent such as ether, is added to the Grignard reagent. The temperature should be monitored and not allowed to exceed ˜30° C. The product, a compound of Formula 105, is isolated and purified.

Preparation of Compounds of Formula 107

Referring to Reaction Scheme 1, Step 3, lithium bis(trimethylsilyl)amide (about 3.3 equivalents) is added slowly over ˜3 minutes to a −78° C. solution of a compound of Formula 105 in a nonpolar, aprotic solvent such as THF. The reaction solution temperature should be monitored and the addition of base conducted at a rate sufficient to prevent the temperature from exceeding about −54° C. After the addition is complete, the resulting solution is maintained at −78° C. for about 30 minutes. An acid chloride of Formula 106 (preferably, neat) is then added. The reaction solution is maintained at −78° C. for about 30 minutes. The product is isolated and used without further purification.

A mixture of the above crude product, a base such as potassium carbonate, and a polar, aprotic solvent such as DMF is maintained at about room temperature for about 30 minutes. The product, a compound of Formula 107 is isolated and purified.

Preparation of Compounds of Formula 109

Referring to Reaction Scheme 1, Step 4, optionally, the protecting group, PG, may be removed from the amine. One of skill in the art will appreciate that the conditions for removal of the protecting group will vary with different protecting groups. Such conditions are well known in the art and can be found, e.g., in Greene et al. supra. When PG is Boc, it may be removed by treatment of a compound of Formula 107 with a mixture of aqueous TFA (such as TFA:H₂O, 97.5:2.5) at room temperature. The product, a compound of Formula 109, is isolated and purified.

Preparation of Optically Active Compounds

In compounds of the invention where R₂ differs from R_{2′}, a particular stereo configuration (such as the (R) isomer) may be preferred at the stereogenic center to which R₂ and R₂ are attached. The optically active compound can be prepared by methods known in the art. For example, an amine of Formula 109 is dissolved in an inert organic solvent (such as IPA) and warmed to 60° C. In a separate vessel, a resolving agent (such as dibenzoyl-D-tartaric acid) is dissolved, typically in the same warm solvent, and then quickly added (with agitation) to the warm amine solution. The reaction mixture is left to crystallize by cooling to room temperature over 16 hours under continuing agitation. The desired isomer, e.g., the (R) isomer of a compound of Formula 109, is isolated and purified.

For the sake of brevity in the remaining description of the synthesis of compounds of Formula I, II, or III, it should be understood that either a single isomer or a mixture of isomers may be employed to give the corresponding product. 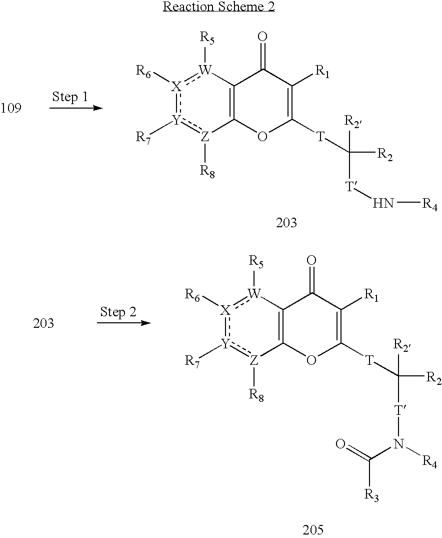
Preparation of Formula 203

Referring to Reaction Scheme 2, Step 1, to a solution of a compound of Formula 109 is added successively a slight excess (such as about 1.2 equivalents) of an aldehyde comprising R₄ (i.e., a compound having the formula R₄.CHO where R₄.CH₂— is equivalent to R₄ and R₄ is as described herein or is a protected precursor to such a substituent, e.g., (3-oxo-propyl)-carbamic acid tert-butyl ester) and a reducing agent such as sodium triacetoxyborohydride. The resulting mixture is stirred for several hours. The product, a compound of Formula 203 is isolated and purified.

Preparation of Formula 205

Referring to Reaction Scheme 2, Step 2, to a solution of a compound of Formula 203 and an amine base such as diisopropylethylamine in a nonpolar, aprotic solvent such as dichloromethane is added an R₃ acyl chloride (such as Cl—C(O)—R₃ where R₃ is as described herein). The resulting solution is stirred under nitrogen at room temperature for several hours. The product, a compound of Formula 205 is isolated and purified.

Optionally, any protecting groups on compounds of Formula 205 are then removed. For example, if R₄ comprises a protected amine wherein the protecting group is a Boc group, the Boc group can be removed by treatment of the compound of Formula 205 with an acid such as trifluoroacetic acid in a nonpolar, aprotic solvent such as dichloromethane, while maintaining the reaction at about room temperature. The reaction is monitored e.g., by TLC. Upon completion, the product is isolated and purified.

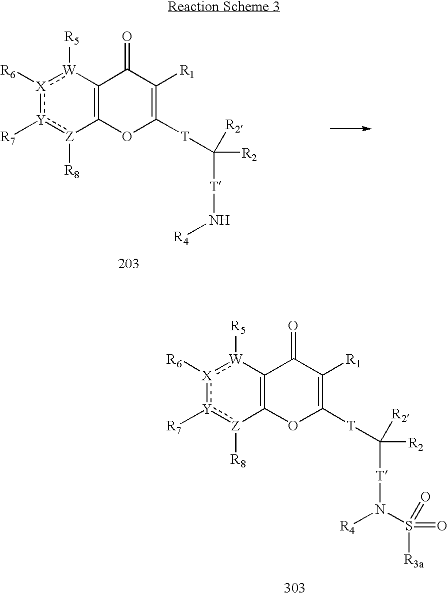

Referring to Reaction Scheme 3, to a solution of a compound of Formula 203 and an amine base such as diisopropylethylamine in a nonpolar, aprotic solvent such as dichloromethane is added a compound having the formula Cl—S(O)₂—R₃ₐ or O—(S(O)₂—R₃ₐ)₂ where R₃ₐ is as described herein. The resulting solution is stirred under nitrogen at room temperature for several hours. The product, a compound of Formula 303 is isolated and purified.

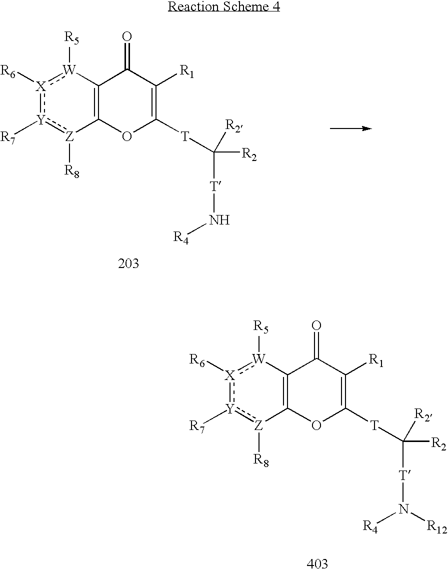

Referring to Reaction Scheme 4, to a solution of a compound of Formula 203 and an amine base such as diisopropylethylamine in a nonpolar, aprotic solvent such as dichloromethane is added a compound having the formula X—R₁₂ where R₁₂ is as described herein and X is a leaving group such as Br, Cl, mesylate, or tosylate. The resulting solution is stirred under nitrogen at room temperature or with heat for several hours. The product, a compound of Formula 403 is isolated and purified. 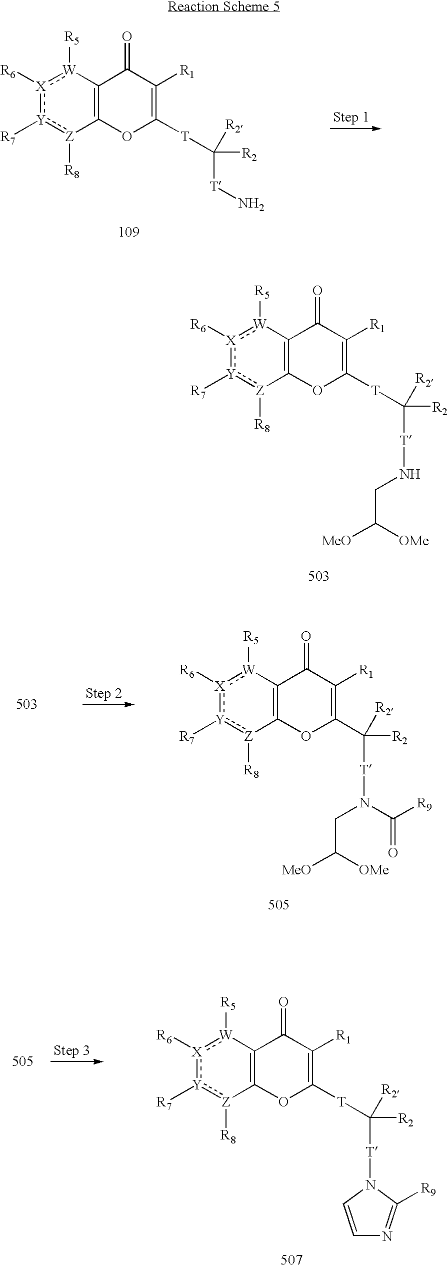
Preparation of Formula 503

Referring to Reaction Scheme 5, Step 1, to an optionally substituted compound of Formula 109 dissolved in a polar, aprotic solvent (such as DMF) in the presence of a base (such as potassium carbonate) is added one equivalent of an optionally substituted suitably protected aldehyde wherein such aldehyde further comprises a leaving group, such as, a halide (for example, bromoacetaldehyde dimethylacetal). The solution is heated at reflux, monitoring completion of the reaction (e.g., by TLC). The reaction mixture is cooled and the corresponding, optionally substituted compound of Formula 503 is isolated and purified.

Preparation of Formula 505

Referring to Reaction Scheme 5, Step 2, to an optionally substituted compound of Formula 503 in an inert solvent (such as dichloromethane) in the presence of about 1.5 molar equivalents of an amine base (such as triethylamine) is added about 1.5 molar equivalents of an R₉ acid chloride, such as, Cl—C(O)—R₉, where R₉ is as described herein. The reaction takes place, with stirring, at room temperature over a period of 4 to 24 hours. Completion is monitored, e.g., by TLC. The corresponding compound of Formula 505 is isolated and purified.

Preparation of Formula 507

Referring to Reaction Scheme 5, Step 3, a solution of a compound of Formula 505 and an excess of ammonium acetate in acetic acid is heated at reflux for 1-4 hours. Completion is monitored, e.g., by TLC. The corresponding compound of Formula 507 is isolated and purified. 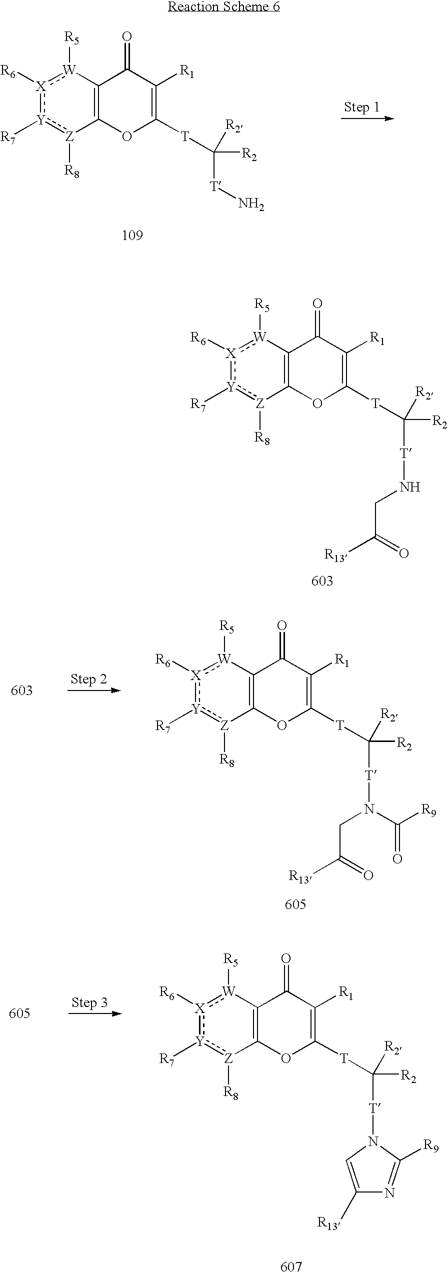
Preparation of Formula 603

Referring to Reaction Scheme 6, Step 1, a suspension of a compound of Formula 109, an alpha-haloketone reagent of the Formula R_{13′}(CO)CH₂X wherein X is a halide and R₁₃ is as described herein, and about an equivalent of a base, such as potassium carbonate in a polar, aprotic solvent such as DMF is stirred at room temperature. The reaction is diluted with water and the resulting compound, a compound of Formula 603, typically a sold, is used in the subsequent step without purification. Where the resulting compound is not a solid, it is isolated using standard procedures and then used in the subsequent step.

Preparation of Formula 605

Referring to Reaction Scheme 6, Step 2, a solution of the compound of Formula 603, about an equivalent of an amine base, such as triethylamine and about an equivalent of an acid chloride (such as a compound of Formula R₉—COCl) in an organic solvent such as methylene chloride is stirred at room temperature for several hours. Completion is monitored, e.g., by TLC. The corresponding compound of Formula 605 is isolated and purified.

Preparation of Formula 607

Referring to Reaction Scheme 6, Step 3, a solution of a compound of Formula 605 and an excess of ammonium acetate in acetic acid is heated at reflux using a Dean-Stark trap and condenser. Completion is monitored, e.g., by TLC. The corresponding compound of Formula 607 is isolated and purified.

If R_{13′} comprises a protected aminoalkyl group, the amino protected group may be removed. For example, when the amino group is protected as the corresponding phthalimide, the protecting group is removed as follows. A solution of a compound of Formula 607 and an excess of anhydrous hydrazine in a polar, protic solvent such as ethanol is heated at reflux. The reaction is cooled to about 5° C. and any precipitate is filtered off. The filtrate is concentrated in vacuo and purified to yield the corresponding free amine. One of skill in the art will appreciate that other conditions may be used to remove other protecting groups. 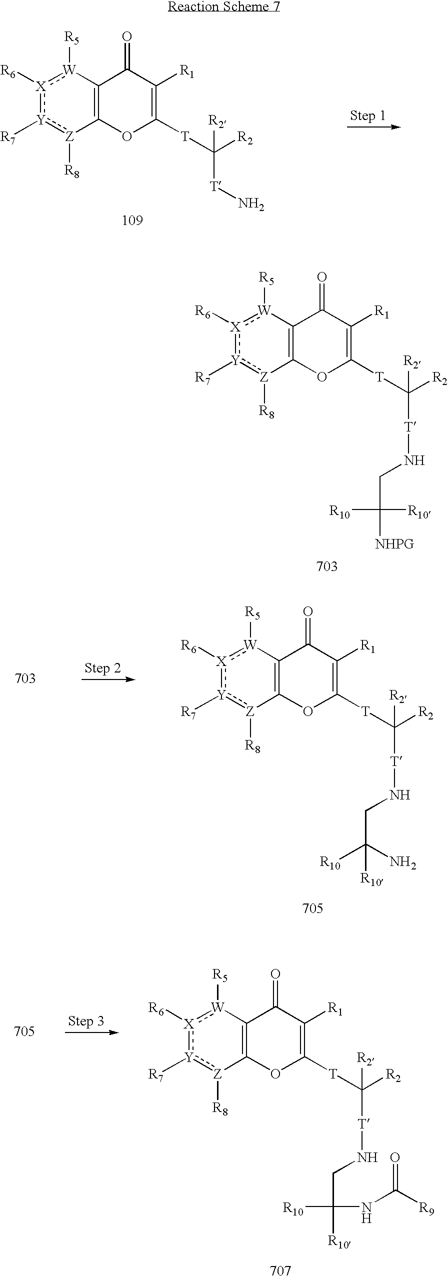 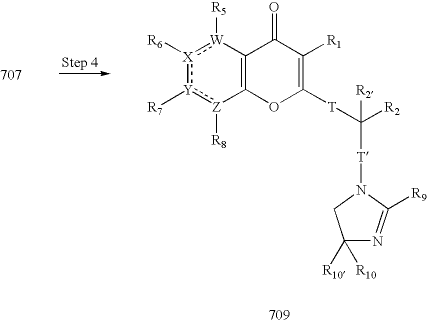
Preparation of Formula 703

Referring to Reaction Scheme 7, Step 1, reductive amination of amines of Formula 109 with an optionally substituted, aldehyde-containing carbamic acid ester gives urethane intermediates. More specifically, to a solution of a compound of Formula 109 and an equivalent of a suitably protected aldehyde (Seki et. al. Chem. Pharm. Bull. 1996, 44, 2061) in dichloromethane is added a slight excess of a reducing agent, such as sodium triacetoxyborohydride. The resultant cloudy mixture is maintained at ambient temperature. Completion is monitored, e.g., by TLC. The corresponding compound of Formula 703 is isolated and used in the subsequent step without purification.

Preparation of Formula 705

Referring to Reaction Scheme 7, Step 2, the amino protecting group PG is then removed. When PG is a Boc protecting group, this may be accomplished by the treatment of a solution of a compound of Formula 703 in a nonpolar, aprotic solvent such as dichloromethane with a strong acid such as trifluoroacetic acid. The resultant solution is maintained at ambient temperature overnight and concentrated under reduced pressure. The residue is isolated to give a compound of Formula 705 which was used in the subsequent step without purification.

Preparation of Formula 707

Referring to Reaction Scheme 7, Step 3, to a solution of a compound of Formula 705 in a nonpolar, aprotic solvent such as dichloromethane is added an excess, such as about two equivalents, of an amine base such as triethylamine, followed by about an equivalent or slight excess of an acid chloride of the formula R₉—CO—Cl. The resultant solution is stirred at ambient temperature for about 3 hours. Completion is monitored, e.g., by TLC. The corresponding compound of Formula 707 is isolated and purified.

Preparation of Formula 709

Referring to Reaction Scheme 7, Step 4, a solution of a compound of Formula 707 in an excess of phosphorus oxychloride is heated at reflux. After 8 hours, the reaction mixture is allowed to cool to ambient temperature and concentrated under reduced pressure. The corresponding compound of Formula 709 is isolated and purified. 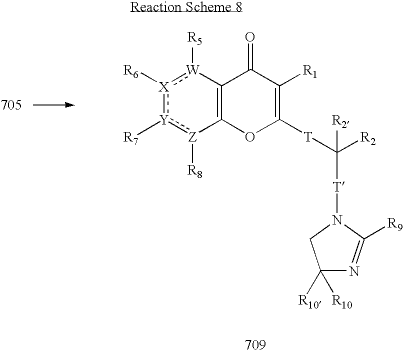
Preparation of Formula 709

As an alternative to Steps 3 and 4 of Reaction Scheme 7, acylation of primary amines of Formula 705, followed by acetic acid mediated cyclization, can proceed without isolation of the intermediate amides to provide the target compound of Formula 709. This route is shown in Reaction Scheme 8.

More specifically, to a solution of a compound of Formula 705 in a nonpolar, aprotic solvent such as dichloromethane is added an excess, such as about two equivalents of an amine base, such as triethylamine, followed by about an equivalent of an acid chloride of the formula R₉—CO—Cl. The resultant solution is stirred at ambient temperature for 2 hours, then evaporated under reduced pressure. The resultant solid is treated with glacial acetic acid, then the resultant suspension is heated at reflux for about 48 hours. The reaction is cooled to ambient temperature then evaporated under reduced pressure. The corresponding compound of Formula 709 is isolated and purified.

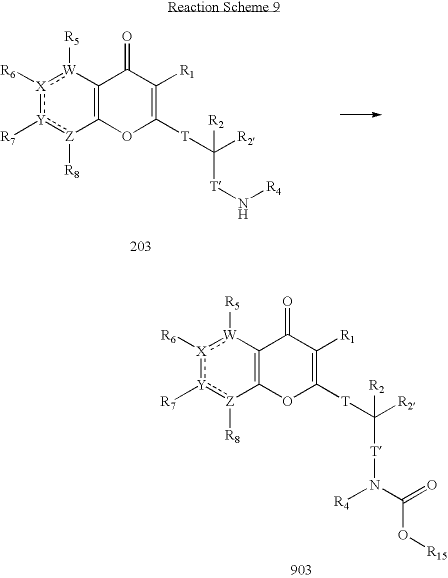

Referring to Reaction Scheme 9, a compound of Formula 203 is reacted with a slight excess of a compound of the formula R₁₅O(CO)Cl in the presence of a base such as triethylamine in a nonpolar, aprotic solvent such as dichloromethane. The product, a compound of Formula 903 is isolated and purified.

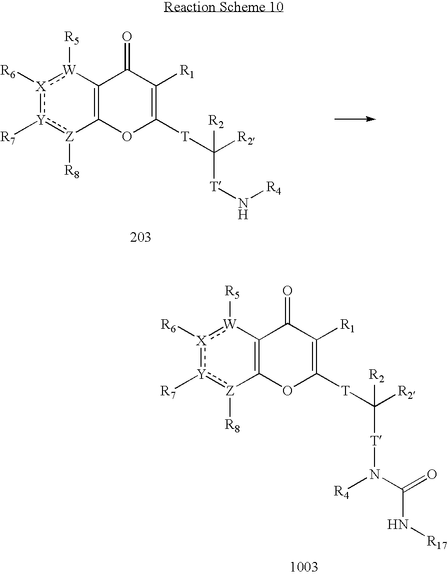

Referring to Reaction Scheme 10, a compound of Formula 203 is treated with a slight excess of an isocyanate R₁₇—N═C═O in the presence of a base, such as triethylamine, in a nonpolar, aprotic solvent, such as dichloromethane. The product, a compound of Formula 1003, is isolated and purified. 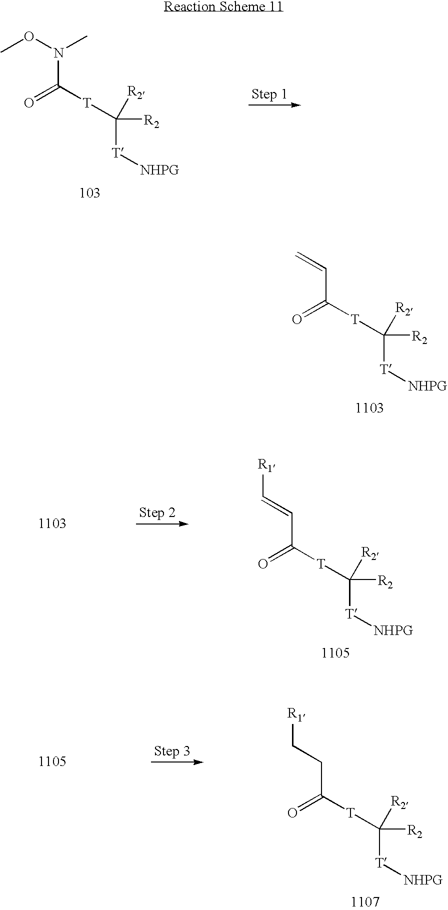
Preparation of Compounds of Formula 1103

Referring to Reaction Scheme 11, Step 1, a nonpolar, aprotic solvent, such as THF, and an excess of a solution of an optionally substituted vinyl magnesium bromide in a nonpolar, aprotic solvent (such as, about three equivalents of a 1.0 M solution of an optionally substituted vinyl magnesium bromide in THF) is cooled to −78° C. while stirring under a nitrogen atmosphere. The mixture is treated dropwise with a solution of a compound of Formula 1101 in a nonpolar, aprotic solvent, such as THF over about 30 min. After the mixture is stirred for 30 min at −78° C., the cooling bath is removed and the reaction mixture is warmed slowly to room temperature overnight (about 15 h). The product, a compound of Formula 1103, is isolated and purified.

Preparation of Compounds of Formula 1105

Referring to Reaction Scheme 11, Step 2, to a solution of a compound of Formula 1103 in an anhydrous, nonpolar, aprotic solvent, such as acetonitrile under an inert atmosphere, such as argon, is added about an equivalent of a compound of the Formula R_{1′}—X wherein R_{1′} is an optionally susbituted vinyl, optionally susbituted aryl, or optionally substituted heteroaryl and X is I, Br, or —OTf, and a base such as triethylamine followed by palladium (II) acetate (such as, about 0.025 equivalents). The resulting solution is heated to about 80° C. After about 15 h, the reaction mixture is allowed to cool to room temperature. The product, a compound of Formula 1105, is isolated and immediately purified.

Preparation of Compounds of Formula 1107

To a solution of a compound of Formula 1105 in a nonpolar, aprotic solvent such as ethyl acetate under nitrogen is added 10 wt % palladium on carbon. The nitrogen is replaced with a balloon of hydrogen and the flask is purged. After 3 h, the reaction flask is purged with nitrogen and filtered through a pad of celite (rinsing with a solvent such as ethyl acetate). The product, a compound of Formula 1107 is isolated and purified. 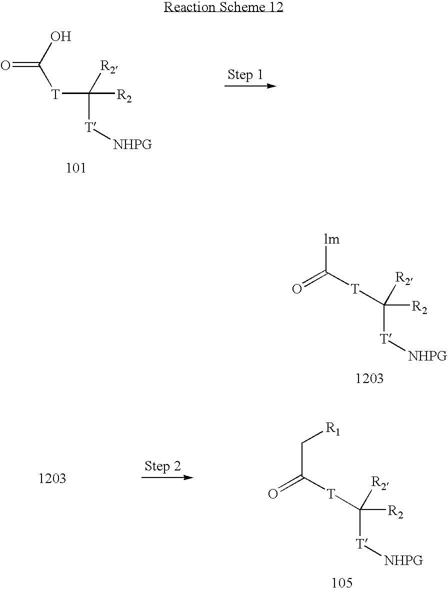
Preparation of Compounds of Formula 1203

Referring to Reaction Scheme 12, Step 1, about one equivalent of carbonyldiimidazole is added slowly to a room temperature solution of a compound of Formula 101 (for example, wherein the amino protecting group PG is Boc) in a nonpolar, aprotic solvent such THF. Afer about one hour, the product, a compound of Formula 1203, is isolated and used without further purification.

Preparation of Compounds of Formula 1205

Referring to Reaction Scheme 12, Step 2, a Grignard reagent is prepared from a compound of Formula R₁CH₂Br and magnesium turnings in a nonpolar, aprotic solvent such as THF. A solution of a compound of Formula 1203 in a nonpolar, aprotic solvent such as THF is cooled to about 0-5° C. The solution of the Grignard reagent is then added via syringe to the 0-5° C. solution of the compound of Formula 1203. The temperature is monitored by internal thermometer and is not allowed to exceed about 15° C. The reaction mixture is maintained at about 0-5° C. for about one hour. The product, a compound of Formula 1205, is isolated and purified.

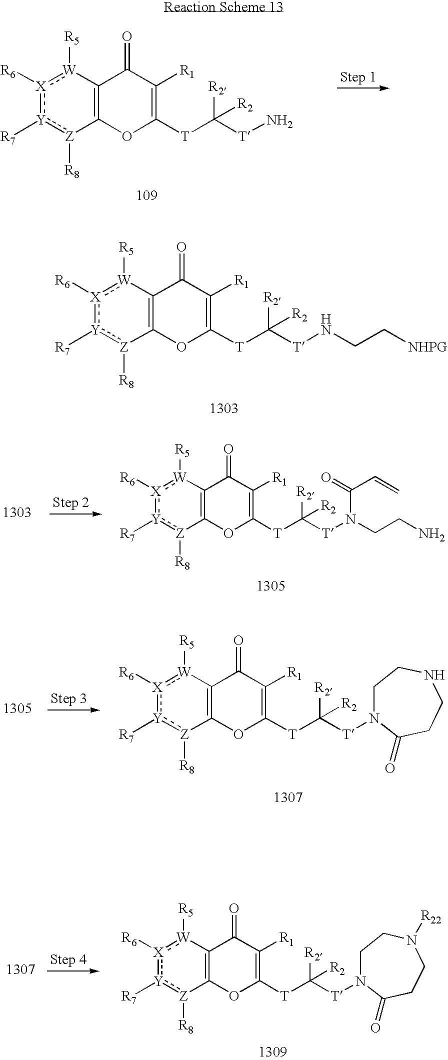

Referring to Reaction Scheme 13, reductive amination of the primary amino group in compounds of Formula 109 with (2-oxo-ethyl)-carbamic acid tert-butyl ester gave the corresponding secondary amine. Acylation with acryloyl chloride followed by deprotection of the tertiary amide and base mediated cyclisation gave the desired diazepanones. If desired, further functionalization of the basic amine could be accomplished under conditions well known to those skilled in the art.

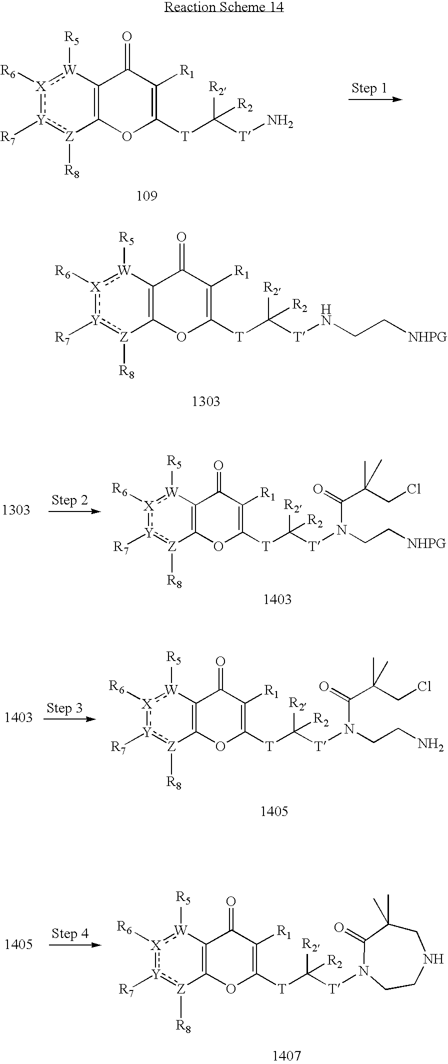

Referring to Reaction Scheme 14, reductive amination of the primary amino group in compounds of Formula 109 with (2-oxo-ethyl)-carbamic acid tert-butyl ester gave the corresponding secondary amine. Acylation with chloropivaloyl chloride followed by deprotection of the tertiary amide and base mediated cyclisation gave the desired diazepanones. If desired, further functionalization of the basic amine could be accomplished under conditions well known to those skilled in the art.

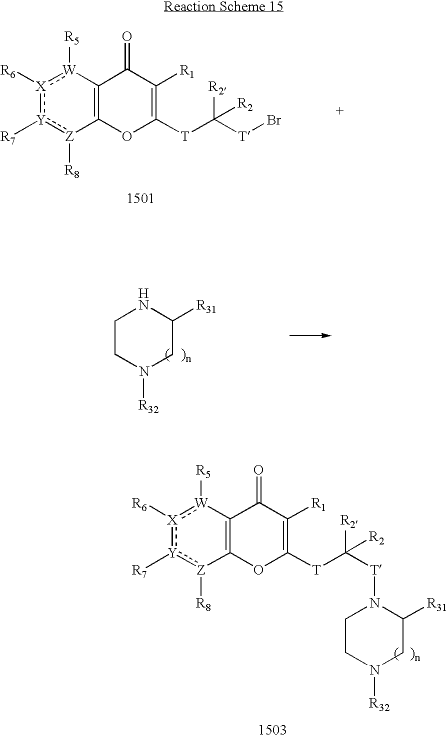

Referring to Reaction Scheme 15, a compound of Formula 1501, one-half molar equivalent of an optionally substituted piperazine or diazepam (as shown above, where R₃₂ is as described herein) and an excess of potassium carbonate are combined in an organic solvent (e.g., acetonitrile). The reaction takes place under a nitrogen atmosphere at elevated temperature (e.g., 100° C.) over a period of 8 hours, followed at a somewhat lower temperature (e.g., 60° C.) for a period of 5 days. The product, a compound of Formula 1503, is isolated and purified.

Optionally, in the event that R₃₂ is an amine protecting group, such as Boc, it may be removed by for example treatment with a 95/5 mixture of TFA/water followed by stirring at room temperature for 1 hour. The product, a compound of Formula 1803 wherein R₃₂ is hydrogen, can be isolated and purified. If desired, further functionalization of the basic amine could be accomplished under conditions well known to those skilled in the art.

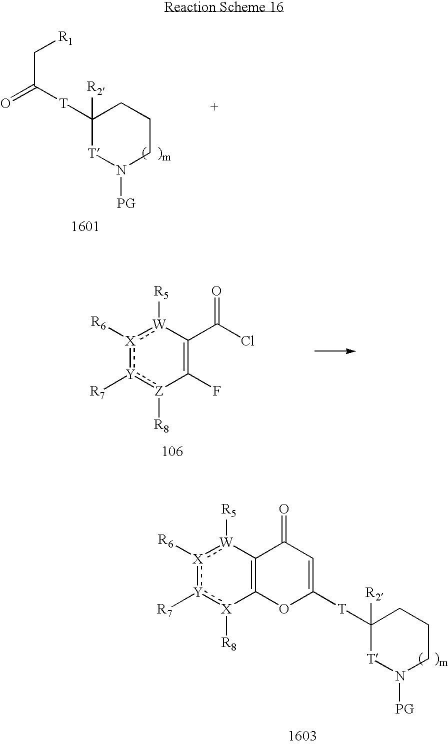

The synthesis of compounds of Formula 1 wherein R₁₂ taken together with R₂ form an optionally substituted 5- to 12-membered nitrogen-containing heterocycle can be accomplished according to the general procedure shown in Scheme 16 and as described further in Reaction Scheme 1 above. The requisite ketone of Formula 1601 can be prepared using methods well known to those skilled in the art, for example, as described by Quintero et al (1991), Canadian Journal of Chemistry, 2059-2063.

### Processes and Last Steps

A compound of Formula I, II, or III is optionally contacted with a pharmaceutically acceptable acid or base to form the corresponding acid or base addition salt.

A pharmaceutically acceptable acid addition salt of a compound of Formula I, II, or III is optionally contacted with a base to form the corresponding free base of Formula I, II, or III.

A pharmaceutically acceptable base addition salt of a compound of Formula I, II, or III is optionally contacted with an acid to form the corresponding free acid of Formula I, II, or III.

### Particular Embodiments of Compounds of the Invention

T and T′

When considering the compounds of Formula I or Formula II, T is optionally substituted alkylene or is a covalent bond; and T′ is optionally substituted alkylene or is a covalent bond. In some embodiments of Formula I or Formula II, one of T and T′ is a covalent bond and the other is optionally substituted alkylene (such as optionally substituted methylene). In some embodiments of Formula I or Formula II, both of T and T′ are independently optionally substituted alkylene. In some embodiments of Formula I, both of T and T′ are covalent bonds.

W, X, Y, and Z

When considering the compounds of Formula I, W, X, Y, and Z are independently N, C, O, and S, and Z is optionally absent. In some embodiments, the ring comprising W, X, Y, and optionally Z is heteroaromatic. In some embodiments, at least one of W, X, Y, or Z is other than C. In some embodiments, no more than two of W, X, Y, and Z are —N═. In some embodiments, W, X, or Y can be O or S only when Z is absent. In some embodiments, the ring comprising W, X, Y, and optionally Z is heteroaromatic; at least one of W, X, Y, and Z is not C; no more than two of W, X, Y, and Z are —N═; and W, X, or Y is O or S only when Z is absent.

In some embodiments, one of W, X, Y, and Z is N and the others are C. In some embodiments, two of W, X, Y, and Z are N with the others being C. In some embodiments, the ring incorporating W, X, Y, and optionally Z is an optionally substituted pyridinyl-, pyrimidinyl-, pyridazinyl, pyrazinyl, imidazolyl, isoxazolyl, isothiazolyl, pyrazolyl-, thiazolyl-, oxazolyl, furanyl, pyrrolyl, or thiophenyl ring.

R₁

When considering the compounds of Formula I, II, or III, in some embodiments, R₁ is selected from hydrogen, optionally substituted C₁-C₈ alkyl, optionally substituted aryl, optionally substituted heteroaryl, optionally substituted aryl-C₁-C₄-alkyl-, and optionally substituted heteroaryl-C₁-C₄-alkyl- (such as optionally substituted aryl and optionally substituted aryl-C₁-C₄-alkyl-). In some embodiments, R₁ is selected from hydrogen, optionally substituted C₁-C₄ alkyl, optionally substituted phenyl-C₁-C₄-alkyl-, optionally substituted heteroaryl-C₁-C₄-alkyl-, optionally substituted naphthalenylmethyl, optionally substituted phenyl, and naphthyl. In some embodiments, R₁ is optionally substituted phenyl-C₁-C₄-alkyl-, optionally substituted heteroaryl-C₁-C₄-alkyl-, optionally substituted naphthalenylmethyl, optionally substituted phenyl, or naphthyl (such as optionally substituted phenyl-C₁-C₄-alkyl- or optionally substituted heteroaryl-C₁-C₄-alkyl-).

In some embodiments, R₁ is naphthyl, phenyl, bromophenyl, chlorophenyl, methoxyphenyl, ethoxyphenyl, tolyl, dimethylphenyl, chlorofluorophenyl, methylchlorophenyl, ethylphenyl, phenethyl, benzyl, halobenzyl (such as chlorobenzyl or bromobenzyl), methylbenzyl, methoxybenzyl, cyanobenzyl, hydroxybenzyl, dichlorobenzyl, dimethoxybenzyl, or naphthalenylmethyl. In some embodiments, R₁ is benzyl, halobenzyl, methylbenzyl, hydroxybenzyl, cyanobenzyl, methoxybenzyl, or naphthalenylmethyl. In some embodiments, R₁ is benzyl.

R₂

When considering the compounds of Formula I, II, or III, and as will be appreciated by those skilled in the art, the compounds described herein possess a potentially chiral center at the carbon to which R₂ and R₂, are attached. The R₂ and R₂ groups may be the same or different; if different, the compound is chiral (i.e., has a stereogenic center). When R₂ and R_{2′} are different, in some embodiments, R_{2′} is hydrogen and R₂ is other than hydrogen. The invention contemplates the use of pure enantiomers and mixtures of enantiomers, including racemic mixtures, although the use of a substantially optically pure enantiomer will often be preferred. The term “substantially optically pure” or “enantiomerically pure” means having at least about 97.5% of the described enantiomer with no single impurity greater than about 1% and some embodiments, at least about 95% enantiomeric excess. In some embodiments, the stereogenic center to which R₂ and R_{2′} are attached is of the R configuration.

When considering the compounds of Formula I, II, or III, in some embodiments, R₂ is optionally substituted C₁-C₄ alkyl, and R_{2′} is hydrogen or optionally substituted C₁-C₄ alkyl. In some embodiments, R_{2′} is hydrogen and R₂ is optionally substituted C₁-C₄ alkyl. In some embodiments, R₂ is chosen from methyl, ethyl, propyl (such as, c-propyl or i-propyl), butyl (such as, t-butyl), methylthioethyl, methylthiomethyl, aminobutyl, (CBZ)aminobutyl, cyclohexylmethyl, benzyloxymethyl, methylsulfinylethyl, methylsulfinylmethyl, and hydroxymethyl, and R_{2′} is hydrogen. In some embodiments, R_{2′} is hydrogen and R₂ is ethyl or propyl (such as, c-propyl or i-propyl). In some embodiments, R₂ is i-propyl. In some embodiments, the stereogenic center to which R₂ and R_{2′} is attached is of the R configuration.

When considering the compounds of Formula I, II, or III, in some embodiments, both R₂ and R_{2′} are hydrogen.

R₂ taken together with R₄

When considering the compounds of Formula I, II, or III, in some embodiments, R₂ and R₄ taken together form a 5- to 12-membered ring that optionally incorporates from one to two additional heteroatoms, selected from N, O, and S in the heterocycle ring and may optionally be substituted with one or more of the following groups: alkyl, aryl, aralkyl, heteroaryl, substituted alkyl, substituted aryl, substituted aralkyl, substituted heteroaryl, hydroxyl, alkoxy, cyano, optionally substituted amino, oxo, or carbamyl.

In some embodiments, R₂ and R₄ taken together form an optionally substituted ring of the formula: 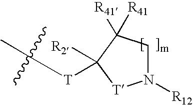
wherein R₄₁ and R_{41′} are independently chosen from hydrogen, alkyl, aryl, aralkyl, heteroaryl, substituted alkyl, substituted aryl, substituted aralkyl, and substituted heteroaryl; m is 0, 1, 2, or 3; and T, T′, R₁₂, and R_{2′} are as defined herein (provided that in compounds of Formula III, T and T′ are absent). In some embodiments, R₄₁ is hydrogen. In some embodiments, both R₄₁ and R_{41′} are hydrogen. In some embodiments, R₁₂ is optionally substituted aralkyl (such as benzyl) or optionally substituted acyl (i.e., R₁₂ is —(CO)R₃ where R₃ is as defined herein, such as where R₃ is optionally subsitutetd phenyl). See, e.g., U.S. Ser. No. 60/414,756, which is incorporated herein by reference for all purposes.

In some embodiments, R₂ and R₄ taken together form an optionally substituted ring of the formula: 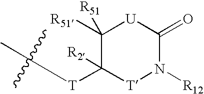
wherein R₁₂, R_{2′}, T, and T′ are as defined herein (provided that in compounds of Formula III, T′ and T are absent); R₅₁ and R_{51′} are independently chosen from hydrogen, alkyl, aryl, aralkyl, heteroaryl, substituted alkyl, substituted aryl, substituted aralkyl and substituted heteroaryl; U is a covalent bond, CR′R″ or NR′″; R′ and R″ are independently chosen from hydrogen, hydroxy, amino, optionally substituted aryl, optionally substituted alkylamino, optionally substituted alkyl and optionally substituted alkoxy; and R′″ is chosen from hydrogen, optionally substituted alkyl, optionally substituted aryl, optionally substituted aralkyl, optionally substituted heteroaryl, and optionally substituted heteroaralkyl.

In some embodiments, R_{51′} is hydrogen or optionally substituted lower alkyl; in some embodiments, R₅₁ is hydrogen. In some embodiments, R_{51′} is hydrogen or optionally substituted lower alkyl; in some embodiments, R_{51′} is hydrogen.

In some embodiments, R₁₂ is optionally substituted aryl or optionally substituted aralkyl; in some embodiments, R₁₂ is optionally substituted phenyl, benzyl or methyl-benzyl (such as benzyl or methyl-benzyl).

In some embodiments, U is CR′R″ where R′ and/or R″ are hydrogen. In some embodiments, U is NR′″ where R′″ is hydrogen or optionally substituted alkyl. In some embodiments, R′″ is hydrogen or optionally substituted amino-lower alkyl. See, e.g., U.S. Ser. No. 60/398,224, which is incorporated herein by reference for all purposes.

R₁₂

In some embodiments, R₁₂ is chosen from optionally substituted C₁-C₁₃ alkyl (such as substituted C₁-C₄ alkyl); optionally substituted aralkyl (such as optionally substituted benzyl or naphthylmethyl-); and optionally substituted heteroaralkyl. In some embodiments, R₁₂ is benzyl or benzyl substituted with one or more of the following groups: carboxy, alkoxycarbonyl, cyano, halo, C₁-C₄ alkyl-, C₁-C₄ alkoxy, nitro, methylenedioxy, or trifluoromethyl.

R₃ Groups When R₁₂ is —C(O)R₃

When considering the compounds of Formula I or II wherein R₁₂ is —C(O)R₃, in some embodiments, R₃ is selected from optionally substituted C₁-C₈ alkyl, optionally substituted aryl-C₁-C₄-alkyl-, optionally substituted heteroaryl-C₁-C₄-alkyl-, optionally substituted heteroaryl, optionally substituted aryl, R₁₅O—, and R₁₇—NH—, where R₁₅ is chosen from optionally substituted C₁-C₈ alkyl and optionally substituted aryl, and R₁₇ is chosen from hydrogen, optionally substituted C₁-C₈ alkyl and optionally substituted aryl.

In some embodiments R₃ is selected from optionally substituted C₁-C₈ alkyl, optionally substituted aryl-C₁-C₄-alkyl-, optionally substituted heteroaryl-C₁-C₄-alkyl-, optionally substituted heteroaryl, and optionally substituted aryl. In some embodiments, R₃ is chosen from phenyl;
phenyl substituted with one or more of the following substituents: halo; C₁-C₄ alkyl; C₁-C₄ alkyl substituted with hydroxy (e.g., hydroxymethyl); C₁-C₄ alkoxy; C₁-C₄ alkyl substituted with C₁-C₄ alkoxy, nitro, formyl, carboxy, cyano, methylenedioxy, ethylenedioxy, acyl (e.g., acetyl), —N-acyl (e.g., N-acetyl), or trifluoromethyl;
benzyl;
phenoxymethyl-;
halophenoxymethyl-;
phenylvinyl-;
heteroaryl;
heteroaryl-substituted with C₁-C₄ alkyl or C₁-C₄ alkyl substituted with halo (e.g., CF₃);
C₁-C₄ alkyl substituted with C₁-C₄ alkoxy-; and
benzyloxymethyl-.

In some embodiments, when R₃ is not R₁₇NH— or R₁₅O—, R₃ is chosen from phenyl, halophenyl, dihalophenyl, cyanophenyl, halo(trifluoromethyl)phenyl, hydroxymethylphenyl, methoxymethylphenyl, methoxyphenyl, ethoxyphenyl, carboxyphenyl, formylphenyl, ethylphenyl, tolyl, methylenedioxyphenyl, ethylenedixoyphenyl, methoxychlorophenyl, dihydro-benzodioxinyl, methylhalophenyl, trifluoromethylphenyl, furanyl, C₁-C₄ alkyl substituted furanyl, trifluoromethylfuranyl, C₁-C₄ alkyl substituted trifluoromethylfuranyl, benzofuranyl, thiophenyl, C₁-C₄ alkyl substituted thiophenyl, benzothiophenyl, benzothiadiazolyl, pyridinyl, indolyl, methylpyridinyl, trifluoromethylpyridinyl, pyrrolyl, quinolinyl, picolinyl, pyrazolyl, C₁-C₄ alkyl substituted pyrazolyl, N-methylpyrazolyl, C₁-C₄ alkyl substituted N-methylpyrazolyl, C₁-C₄ alkyl substituted pyrazinyl, C₁-C₄ alkyl substituted isoxazolyl, benzoisoxazolyl, morpholinomethyl, methylthiomethyl, methoxymethyl, N-methyl imidazolyl, and imidazolyl. In some embodiments, R₃ is optionally substituted phenyl (such as tolyl, halophenyl, methylhalophenyl, hydroxymethylphenyl, halo(trifluoromethyl)phenyl-, methylenedioxyphenyl, formylphenyl or cyanophenyl).

In some embodiments, when R₃ is R₁₇NH—, R₁₇ is chosen from hydrogen, C₁-C₄ alkyl; cyclohexyl; phenyl; and phenyl substituted with halo, C₁-C₄ alkyl, trifluoromethyl, C₁-C₄ alkoxy, or C₁-C₄ alkylthio.

In some embodiments, when R₃ is R₁₇NH—, R₁₇ is hydrogen, isopropyl, butyl, cyclohexyl, phenyl, bromophenyl, dichlorophenyl, methoxyphenyl, ethylphenyl, tolyl, trifluoromethylphenyl or methylthiophenyl.

In some embodiments wherein R₃ is R₁₅O—, R₁₅ is chosen from optionally substituted C₁-C₈ alkyl and optionally substituted aryl.

R₃ₐ Groups when R₁₂ is —SO₂R₃ₐ

In considering compounds of Formula I or II, in some embodiments, when R₁₂ is —SO₂R₃ₐ, R₃ₐ is chosen from C₁-C₁₃ alkyl; phenyl; naphthyl; phenyl substituted with halo, C₁-C₄ alkyl, C₁-C₄ alkoxy, cyano, nitro, methylenedioxy, or trifluoromethyl; biphenylyl; and heteroaryl. In some embodiments, R₃ₐ is chosen from naphthyl and phenyl substituted with halo, C₁-C₄ alkyl, C₁-C₄ alkoxy, cyano, nitro, methylenedioxy, and/or trifluoromethyl.

R₄ Groups

When considering compounds of Formula I, II, or III, in some embodiments, R₄ is chosen from hydrogen, optionally substituted C₁-C₁₃ alkyl, optionally substituted aryl, optionally substituted aryl-C₁-C₄-alkyl-, optionally substituted heterocyclyl, and optionally substituted heteroaryl-C₁-C₄-alkyl- (such as hydrogen or optionally substituted C₁-C₁₃ alkyl).

In some embodiments, R₄ is chosen from hydrogen; C₁-C₄ alkyl; cyclohexyl; phenyl substituted with hydroxyl, C₁-C₄ alkoxy, or C₁-C₄ alkyl; benzyl; and R₁₆-alkylene-, wherein R₁₆ is hydroxyl, carboxy, (C₁-C₄ alkoxy)carbonyl-, di(C₁-C₄ alkyl)amino-, (C₁-C₄ alkyl)amino-, amino, (C₁-C₄ alkoxy)carbonylamino-, C₁-C₄ alkoxy-, optionally substituted furanyl, or optionally substituted N-heterocyclyl- (such as azetidinyl, morpholinyl, pyridinyl, indolyl, pyrrolidinyl, piperidinyl, or imidazolyl, each of which may be optionally substituted).

In some embodiments, R₄ is chosen from hydrogen, methyl, ethyl, propyl, butyl, cyclohexyl, carboxyethyl, carboxymethyl, methoxyethyl, hydroxyethyl, hydroxypropyl, dimethylaminoethyl, dimethylaminopropyl, diethylaminoethyl, diethylaminopropyl, aminopropyl, methylaminopropyl, 2,2-dimethyl-3-(dimethylamino)propyl, aminoethyl, aminobutyl, aminopentyl, aminohexyl, isopropylaminopropyl, diisopropylaminoethyl, 1-methyl-4-(diethylamino)butyl, (t-Boc)aminopropyl, hydroxyphenyl, benzyl, methoxyphenyl, methylmethoxyphenyl, dimethylphenyl, tolyl, ethylphenyl, (oxopyrrolidinyl)propyl, (methoxycarbonyl)ethyl, benzylpiperidinyl, pyridinylethyl, pyridinylmethyl, morpholinylethyl, morpholinylpropyl, piperidinyl, azetidinylmethyl, azetidinylethyl, azetidinylpropyl, pyrrolidinylethyl, pyrrolidinylpropyl, piperidinylmethyl, piperidinylethyl, imidazolylpropyl, imidazolylethyl, (ethylpyrrolidinyl)methyl, (methylpyrrolidinyl)ethyl, (methylpiperidinyl)propyl, (methylpiperazinyl)propyl, furanylmethyl, and indolylethyl.

In some embodiments, R₄ is R₁₆-alkylene-, wherein R₁₆ is amino, C₁-C₄ alkylamino-, di(C₁-C₄ alkyl)amino-, C₁-C₄ alkoxy-, hydroxyl, or N-heterocyclyl. In some embodiments, R₁₆ is amino. In some embodiments, the alkylene moiety of R₁₆-alkylene- has from 1 to 6 carbon atoms.

In some embodiments, R₄ is aminoethyl, aminopropyl, aminobutyl, aminopentyl, aminohexyl, methylaminoethyl, methylaminopropyl, methylaminobutyl, methylaminopentyl, methylaminohexyl, dimethylaminoethyl, dimethylaminopropyl, dimethylaminobutyl, dimethylaminopentyl, dimethylaminohexyl, ethylaminoethyl, ethylaminopropyl, ethylaminobutyl, ethylaminopentyl, ethylaminohexyl, diethylaminoethyl, diethylaminopropyl, diethylaminobutyyl, diethylaminopentyl, or diethylaminohexyl, and in some embodiments, aminopropyl.

R₁₂ taken together with R₄

When considering the compounds of Formula I, II, or III, in some embodiments, R₄ taken together with R₁₂, and the nitrogen to which they are bound, form an optionally substituted 5- to 12-membered nitrogen-containing heterocycle, which optionally incorporates from one to two additional heteroatoms, selected from N, O, and S, in the heterocycle ring.

When considering the compounds of Formula I or II, in some embodiments, R₄ taken together with R₁₂ and the nitrogen to which they are bound, form an optionally substituted imidazolyl ring of the formula: 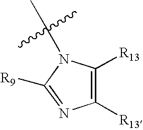
wherein

R₉ is chosen from hydrogen, optionally substituted C₁-C₈ alkyl, optionally substituted aryl, optionally substituted aryl-C₁-C₄-alkyl-, optionally substituted heteroaryl-C₁-C₄-alkyl-, optionally substituted aryl-C₁-C₄-alkoxy-, optionally substituted heteroaryl-C₁-C₄-alkoxy-, and optionally substituted heteroaryl-; and R₁₃ and R_{13′} are independently hydrogen, optionally substituted C₁-C₈ alkyl, optionally substituted aryl, or optionally substituted aryl-C₁-C₄-alkyl- (such as optionally substituted alkyl). In some embodiments, R₉ is phenyl substituted with C₁-C₄-alkyl, C₁-C₄-alkoxy-, and/or halo (such as C₁-C₄-alkyl and/or halo); phenyl; or benzyl. In some embodiments, R₉ is tolyl; halophenyl; or halomethylphenyl.

In some embodiments, R₁₃ is hydrogen and R₁₃ is substituted C₁-C₄ alkyl. In some embodiments, R₁₃ is hydrogen and R_{13′} is aminomethyl, aminoethyl, aminopropyl, acetylaminomethyl, acetylaminoethyl, benzyloxycarbonylamino-methyl, or benzyloxycarbonylamino-ethyl. See, e.g., PCT/US03/14787, which is incorporated herein by reference

In some embodiments of Formula I or II, R₁₂ taken together with R₄ forms an optionally substituted imidazolinyl ring of the formula: 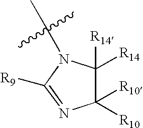
wherein R₉ is chosen from hydrogen, optionally substituted C₁-C₈ alkyl, optionally substituted aryl, optionally substituted aryl-C₁-C₄-alkyl-, and optionally substituted heteroaryl-; and R₁₀, R₁₀, R₁₄, and R₁₄ are independently chosen from hydrogen, optionally substituted C₁-C₈ alkyl, optionally substituted aryl, and optionally substituted aryl-C₁-C₄-alkyl-. In some embodiments, R₉ is methylenedioxyphenyl; phenyl; phenyl substituted with C₁-C₄ alkyl, C₁-C₄ alkoxy, and/or halo; or benzyl. In some embodiments, R₉ is optionally substituted phenyl (such as halophenyl, halomethylphenyl, tolyl, or methylenedioxyphenyl). In some embodiments, R₁₀, R_{10′}, R_{14′}, and R₁₄ are independently hydrogen or optionally substituted alkyl (such as optionally substituted C₁-C₄ alkyl). In some embodiments, R₁₀ and R_{10′} are independently selected from hydrogen and optionally substituted C₁-C₄ alkyl (and in some embodiments, methyl or aminoalkyl-), and R₁₄ and R₁₄ are hydrogen.

When considering the compounds of Formula I, II, or III, in some embodiments, R₄ taken together with R₁₂ forms an optionally substituted diazepinone ring of the formula: 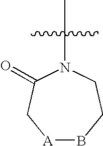
wherein A and B are each independently chosen from C(R₂₀)(R₂₁), N(R₂₂), O, or S, wherein R₂₀ and R₂₁ are each independently selected from H, optionally substituted alkyl, optionally substituted aryl, and optionally substituted heteroaryl; and R₂₂ is H, optionally substituted alkyl, optionally substituted aralkyl, optionally substituted heteroaralkyl, optionally substituted alkylcarbonyl, optionally substituted arylcarbonyl, optionally substituted heteroarylcarbonyl, optionally substituted aralkylcarbonyl, optionally substituted heteroaralkylcarbonyl, optionally substituted alkoxycarbonyl, optionally substituted aryloxycarbonyl, optionally substituted heteroaryloxycarbonyl, optionally substituted aralkyloxycarbonyl, or optionally substituted heteroaralkyloxycarbonyl. In some embodiments, the diazepinone ring is further substituted with one or more of the following groups: optionally substituted alkyl, optionally substituted aryl, optionally substituted aralkyl, optionally substituted heteroaryl, and optionally substituted heteroaralkyl.

In some embodiments of the compounds of Formula I, II, or III, one of A or B is C(R₂₀)(R₂₁), wherein R₂₀ and R₂₁ are each independently selected from H or C₁-C₄ alkyl, and the other of A or B is N(R₂₂), where R₂₂ is H, C₁-C₄ alkyl, optionally substituted aralkyl, optionally substituted heteroaralkyl, C₁-C₆ alkylcarbonyl, optionally substituted arylcarbonyl, optionally substituted heteroarylcarbonyl, optionally substituted aralkylcarbonyl, optionally substituted heteroaralkylcarbonyl, C₁-C₆ alkoxycarbonyl, optionally substituted aryloxycarbonyl, optionally substituted heteroaryloxycarbonyl, optionally substituted aralkyloxycarbonyl, or optionally substituted heteroaralkyloxycarbonyl, where the optionally substituted aryl or heteroaryl groups or moieties are unsubstituted or substituted with one or more substituents selected from C₁-C₄ alkyl, C₁-C₄ haloalkyl, C₁-C₄ alkoxy, C₁-C₄ haloalkoxy, amino, C₁-C₄ alkylamino, di-C₁-C₄ alkylamino, carboxy, C₁-C₄ alkylcarbonyloxy, C₁-C₄ alkoxycarbonyl, carboxamido, C₁-C₄ alkylcarboxamido, aminocarbonyl, C₁-C₄ alkylaminocarbonyl, di-C₁-C₄ alkylaminocarbonyl, cyano, C₁-C₄ alkylcarbonyl, halogen, hydroxyl, mercapto and nitro. In some embodiments, A is C(R₂₀)(R₂₁), wherein R₂₀ and R₂, are each H or C₁-C₄ alkyl, and B is N(R₂₂), where R₂₂ is H, C₁-C₄ alkyl, aralkyl, heteroaralkyl, C₁-C₆ alkylcarbonyl, arylcarbonyl, or heteroarylcarbonyl. In some embodiments of the compounds of Formula I, A is CH₂, and B is N(R₂₂), where R₂₂ is H, methyl, benzyl or acetyl (—C(O)methyl). See, e.g., U.S. Ser. No. 60/435,001, which is incorporated herein by reference for all purposes.

In some embodiments of Formula I, II, or III, R₄ taken together with R₁₂ forms an optionally substituted piperazine- or diazepam of the formula: 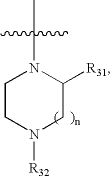
wherein R₃₁ and R₃₂ are independently chosen from hydrogen, optionally substituted alkyl, optionally substituted aryl, optionally substituted heteroaryl, optionally substituted aralkyl, and optionally substituted heteroaralkyl; and n is 1 or 2. In some embodiments, R₃₁ is aryl (such as phenyl), substituted aryl (such as lower alkyl-, lower alkoxy-, and/or halo-substituted phenyl), aralkyl (such as benzyl and phenylvinyl), heteroaralkyl, substituted aralkyl (such as substituted benzyl and substituted phenylvinyl), or substituted heteroaralkyl; R₃₂ is hydrogen; and n is 1. See, e.g., U.S. Ser. No. 60/404,864, which is incorporated herein by reference.
R₅, R₆, R₇, and R₈ Groups

When considering the compounds of Formula I, II, or III, in some embodiments R₅, R₆, R₇, and R₈ are independently chosen from hydrogen; acyl; alkyl; alkyl substituted with alkyl, alkoxy, halo, hydroxyl, nitro, cyano, optionally substituted amino, alkylsulfonyl, alkylsulfonamido, alkylthio, carboxyalkyl, carboxamido, aminocarbonyl, lower-alkylaminocarbonyl- (e.g., methylaminocarbonyl- or ethylaminocarbonyl-), di(lower-alkyl)aminocarbonyl- (e.g., dimethylaminocarbonyl- or diethylaminocarbonyl-), aryl, or heteroaryl; alkoxy; alkoxy substituted with alkyl, acyl, alkoxy, halo, hydroxyl, nitro, cyano, dialkylamino, alkylsulfonyl, alkylsulfonamido, alkylthio, carboxyalkyl, carboxamido, aminocarbonyl, lower-alkylaminocarbonyl- (e.g., methylaminocarbonyl- or ethylaminocarbonyl-), di(lower-alkyl)aminocarbonyl- (e.g., dimethylaminocarbonyl- or diethylaminocarbonyl-), aryl, or heteroaryl; halogen; hydroxyl; nitro; cyano; optionally substituted amino; alkylsulfonyl; alkylsulfonamido; alkylthio; carboxyalkyl; carboxamido; amidocarbonyl; aryl; aryl substituted with alkyl, acyl, alkoxy, halo, hydroxyl, nitro, cyano, dialkylamino, alkylsulfonyl, alkylsulfonamido, alkylthio, carboxyalkyl, carboxamido, aminocarbonyl, lower-alkylaminocarbonyl- (e.g., methylaminocarbonyl- or ethylaminocarbonyl-), di(lower-alkyl)aminocarbonyl- (e.g., dimethylaminocarbonyl- or diethylaminocarbonyl-), aryl, or heteroaryl; heteroaryl or heteroaryl substituted with alkyl, acyl, alkoxy, halo, hydroxyl, nitro, cyano, dialkylamino, alkylsulfonyl, alkylsulfonamido, alkylthio, carboxyalkyl, carboxamido, aminocarbonyl, lower-alkylaminocarbonyl- (e.g., methylaminocarbonyl- or ethylaminocarbonyl-), di(lower-alkyl)aminocarbonyl- (e.g., dimethylaminocarbonyl- or diethylaminocarbonyl-), aryl, or heteroaryl. In compounds of Formula I, R₅, R₆, R₇, and R₅ is absent where W, X, Y, or Z, respectively, is —N═, O, S, or absent.

In some embodiments, R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano. In some embodiments, R₅, R₆, R₇, and R₈ are methoxy, hydrogen, cyano, or halo (such as chloro or fluoro). In some embodiments: R₅ is amino, alkylamino, trifluoromethyl, hydrogen, or halo; R₆ is hydrogen, alkyl (particulary methyl), or halo; R₇ is hydrogen, halo, alkyl (such as methyl), alkoxy (such as methoxy), cyano, or trifluoromethyl; and R₈ is hydrogen or halo. In some embodiments, only one of R₅, R₆, R₇, and R₈ is not hydrogen; in some embodiments, R₇ is not hydrogen. In some embodiments, R₅, R₆, and R₈ are hydrogen and R₇ is cyano, methoxy, or halogen (such as Cl, F).

Salt Forms

Compounds of the invention will generally be capable of forming acid addition salts (i.e., will comprise a site that reacts with a pharmaceutically acceptable acid to form an acid addition salt.) The present invention includes pharmaceutically acceptable acid addition salts of the compounds of Formula I, II, or III. Acid addition salts of the present compounds are prepared in a standard manner in a suitable solvent from the parent compound and an excess of an acid, such as hydrochloric, hydrobromic, sulfuric, phosphoric, acetic, maleic, succinic or methanesulfonic.

The salts and/or solvates of the compounds of Formula I, II, or III that are not pharmaceutically acceptable may be useful as intermediates in the preparation of pharmaceutically acceptable salts and/or solvates of compounds of Formula I, II, or III or the compounds of Formula I, II, or III themselves, and as such form another aspect of the present invention.

Particular Subgenus

Compounds of Formula I

When considering the compounds of Formula I, in some embodiments, one of W, X, Y, and Z is N and the others are C; R₁ is benzyl, halobenzyl, methylbenzyl, hydroxybenzyl, methoxybenzyl-, cyanobenzyl, or naphthalenylmethyl-; R₂ is optionally substituted C₁-C₄ alkyl; R_{2′} is hydrogen; R₅, R₆, R₇, and R₉ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano; T and T′ are independently a covalent bond or optionally substituted lower alkylene (such as where T and T′ are both covalent bonds); R₁₂ is —C(O)R₃ wherein R₃ is tolyl, halophenyl, methylhalophenyl-, hydroxymethylphenyl, halo(trifluoromethyl)phenyl-, methylenedioxyphenyl-, formylphenyl, or cyanophenyl; and R₄ is R₁₆-alkylene-, wherein R₁₆ is amino, C₁-C₄ alkylamino-, di(C₁-C₄ alkyl)amino-, C₁-C₄ alkoxy-, hydroxyl, or N-heterocyclyl. In some embodiments, R₂ is propyl (such as i- or c-propyl).

In some embodiments of compounds of Formula I, one of W, X, Y, and Z is N and the others are C; R₁ is benzyl, halobenzyl, methylbenzyl, hydroxybenzyl, cyanobenzyl, methoxybenzyl, or naphthalenylmethyl; R₂ is optionally substituted C₁-C₄ alkyl; R₂ is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano; T and T′ are independently a covalent bond or optionally substituted lower alkylene (such as where T and T′ are both covalent bonds); and R₄ taken together with R₁₂ form an optionally substituted imidazolinyl of the above formula wherein R₁₀, R_{10′}, R₁₄ and R_{14′} are independently hydrogen or optionally substituted alkyl (such as optionally substituted C₁-C₄ alkyl); and R₉ is optionally substituted phenyl (such as halophenyl, halomethylphenyl, tolyl, or methylenedioxyphenyl). In some embodiments, R₂ is propyl (such as i- or c-propyl).

In some embodiments of compounds of Formula I, one of W, X, Y, and Z is N and the others are C; R₁ is benzyl, halobenzyl, methylbenzyl, hydroxybenzyl, cyanobenzyl, methoxybenzyl, or naphthalenylmethyl; R₂ is optionally substituted C₁-C₄ alkyl; R₂ is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano; T and T′ are independently a covalent bond or optionally substituted lower alkylene (such as where T and T′ are both covalent bonds); and R₄ taken together with R₁₂ form an optionally substituted imidazolyl of the above formula wherein R₁₃ is hydrogen and R_{13′} is hydrogen or optionally substituted alkyl (such as optionally substituted C₁-C₄ alkyl); and R₉ is optionally substituted aryl (such as halophenyl, halomethylphenyl, or tolyl). In some embodiments, R₁₃ is hydrogen and R_{13′} is aminomethyl, aminoethyl, aminopropyl, acetylaminomethyl, acetylaminoethyl, benzyloxycarbonylamino-methyl, or benzyloxycarbonylamino-ethyl.

In some embodiments of compounds of Formula I, one of W, X, Y, and Z is N and the others are C; R₁ is benzyl, halobenzyl, methylbenzyl, hydroxybenzyl, cyanobenzyl, methoxybenzyl, or naphthalenylmethyl; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano; T and T′ are independently a covalent bond or optionally substituted lower alkylene (such as where T and T′ are both covalent bonds); and R₄ taken together with R₁₂ form an optionally substituted imidazolidinyl ring.

In some embodiments of compounds of Formula I, one of W, X, Y, and Z is N and the others are C; R₁ is benzyl, halobenzyl, methylbenzyl, hydroxybenzyl, cyanobenzyl, methoxybenzyl, or naphthalenylmethyl; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano; T and T′ are independently a covalent bond or optionally substituted lower alkylene (such as where T and T′ are both covalent bonds); and R₄ taken together with R₁₂ form an optionally substituted piperazinyl ring.

In some embodiments of compounds of Formula I, one of W, X, Y, and Z is N and the others are C; R₁ is benzyl, halobenzyl, methylbenzyl, hydroxybenzyl, cyanobenzyl, methoxybenzyl, or naphthalenylmethyl; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano; T and T′ are independently a covalent bond or optionally substituted lower alkylene (such as where T and T′ are both covalent bonds); and R₄ taken together with R₁₂ form an optionally substituted diazepinoyl ring.

Compounds of Formula II

When considering the compounds of Formula II, in some embodiments, R₁ is benzyl, halobenzyl, methylbenzyl, hydroxybenzyl, methoxybenzyl-, cyanobenzyl, or naphthalenylmethyl-; R₂ is optionally substituted C₁-C₄ alkyl; R₂ is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano; one of T and T′ is a covalent bond and the other is optionally substituted lower alkylene; R₁₂ is —C(O)R₃ wherein R₃ is tolyl, halophenyl, methylhalophenyl-, hydroxymethylphenyl, halo(trifluoromethyl)phenyl-, methylenedioxyphenyl-, formylphenyl, or cyanophenyl; and R₄ is R₁₆-alkylene-, wherein R₁₆ is amino, C₁-C₄ alkylamino-, di(C₁-C₄ alkyl)amino-, C₁-C₄ alkoxy-, hydroxyl, or N-heterocyclyl. In some embodiments, R₂ is propyl (such as i- or c-propyl).

In some embodiments of compounds of Formula II, R₁ is benzyl, halobenzyl, methylbenzyl, hydroxybenzyl, methoxybenzyl-, cyanobenzyl, or naphthalenylmethyl-; R₂ is optionally substituted C₁-C₄ alkyl; R₂ is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano; one of T and T′ is a covalent bond and the other is optionally substituted lower alkylene; and R₄ taken together with R₁₂ form an optionally substituted imidazolinyl of the above formula wherein R₁₀, R_{10′}, R₁₄ and R₁₄ are independently hydrogen or optionally substituted alkyl (such as optionally substituted C₁-C₄ alkyl); and R₉ is optionally substituted phenyl (such as halophenyl, halomethylphenyl, tolyl, or methylenedioxyphenyl). In some embodiments, R₂ is propyl (such as i- or c-propyl).

In some embodiments of compounds of Formula II, R₁ is benzyl, halobenzyl, methylbenzyl, hydroxybenzyl, methoxybenzyl-, cyanobenzyl, or naphthalenylmethyl-; R₂ is optionally substituted C₁-C₄ alkyl; R₂ is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano; one of T and T′ is a covalent bond and the other is optionally substituted lower alkylene; and R₄ taken together with R₁₂ form an optionally substituted imidazolyl of the above formula wherein R₁₃ is hydrogen and R_{13′} is hydrogen or optionally substituted alkyl (such as optionally substituted C₁-C₄ alkyl); and R₉ is optionally substituted aryl (such as halophenyl, halomethylphenyl, or tolyl). In some embodiments, R₁₃ is hydrogen and R_{13′} is aminomethyl, aminoethyl, aminopropyl, acetylaminomethyl, acetylaminoethyl, benzyloxycarbonylamino-methyl or benzyloxycarbonylamino-ethyl.

In some embodiments of compounds of Formula II, R₁ is benzyl, halobenzyl, methylbenzyl, hydroxybenzyl, methoxybenzyl-, cyanobenzyl, or naphthalenylmethyl-; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano; one of T and T′ is a covalent bond and the other is optionally substituted lower alkylene; and R₄ taken together with R₁₂ form an optionally substituted imidazolidinyl ring.

In some embodiments of compounds of Formula II, R₁ is benzyl, halobenzyl, methylbenzyl, hydroxybenzyl, methoxybenzyl-, cyanobenzyl, or naphthalenylmethyl-; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano; one of T and T′ is a covalent bond and the other is optionally substituted lower alkylene; and R₄ taken together with R₁₂ form an optionally substituted piperazinyl ring.

In some embodiments of compounds of Formula II, R₁ is benzyl, halobenzyl, methylbenzyl, hydroxybenzyl, methoxybenzyl-, cyanobenzyl, or naphthalenylmethyl-; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano; one of T and T′ is a covalent bond and the other is optionally substituted lower alkylene; and R₄ taken together with R₁₂ form an optionally substituted diazepinoyl ring.

Compounds of Formula III

When considering the compounds of Formula III, in some embodiments of compounds of Formula III, R₁ is benzyl, halobenzyl, methylbenzyl, hydroxybenzyl, methoxybenzyl, cyanobenzyl, or naphthalenylmethyl; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy, and cyano; and R₄ taken together with R₁₂ form an optionally substituted imidazolidinyl ring.

In some embodiments of compounds of Formula III, R₁ is benzyl, halobenzyl, methylbenzyl, hydroxybenzyl, methoxybenzyl, cyanobenzyl, or naphthalenylmethyl; R₂ is optionally substituted C₁-C₄ alkyl; R₂ is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy and cyano; and R₄ taken together with R₁₂ form an optionally substituted piperazinyl ring.

In some embodiments of compounds of Formula III, R₁ is benzyl, halobenzyl, methylbenzyl, hydroxybenzyl, methoxybenzyl, cyanobenzyl, or naphthalenylmethyl; R₂ is optionally substituted C₁-C₄ alkyl; R₂ is hydrogen; R₅, R₆, R₇, and R₈ are independently chosen from hydrogen, amino, alkylamino, dialkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy and cyano; and R₄ taken together with R₁₂ form an optionally substituted diazepinoyl ring.

### Utility, Testing and Administration

General Utility

Once made, the compounds of the invention find use in a variety of applications involving alteration of mitosis. As will be appreciated by those skilled in the art, mitosis may be altered in a variety of ways; that is, one can affect mitosis either by increasing or decreasing the activity of a component in the mitotic pathway. Stated differently, mitosis may be affected (e.g., disrupted) by disturbing equilibrium, either by inhibiting or activating certain components. Similar approaches may be used to alter meiosis.

In some embodiments, the compounds of the invention are used to inhibit mitotic spindle formation, thus causing prolonged cell cycle arrest in mitosis. By “inhibit” in this context is meant decreasing or interfering with mitotic spindle formation or causing mitotic spindle dysfunction. By “mitotic spindle formation” herein is meant organization of microtubules into bipolar structures by mitotic kinesins. By “mitotic spindle dysfunction” herein is meant mitotic arrest and monopolar spindle formation.

The compounds of the invention are useful to bind to, and/or inhibit the activity of, a mitotic kinesin, KSP. In some embodiments, the KSP is human KSP, although the compounds may be used to bind to or inhibit the activity of KSP kinesins from other organisms. In this context, “inhibit” means either increasing or decreasing spindle pole separation, causing malformation, i.e., splaying, of mitotic spindle poles, or otherwise causing morphological perturbation of the mitotic spindle. Also included within the definition of KSP for these purposes are variants and/or fragments of KSP. See U.S. Pat. No. 6,437,115, hereby incorporated by reference in its entirety. The compounds of the invention have been shown to have specificity for KSP. However, the present invention includes the use of the compounds to bind to or modulate other mitotic kinesins.

The compounds of the invention are used to treat cellular proliferation diseases. Such disease states which can be treated by the compounds, compositions and methods provided herein include, but are not limited to, cancer (further discussed below), autoimmune disease, fungal disorders, arthritis, graft rejection, inflammatory bowel disease, cellular proliferation induced after medical procedures, including, but not limited to, surgery, angioplasty, and the like. Treatment includes inhibiting cellular proliferation. It is appreciated that in some cases the cells may not be in an abnormal state and still require treatment. Thus, in some embodiments, the invention herein includes application to cells or individuals afflicted or subject to impending affliction with any one of these disorders or states.

The compounds, compositions and methods provided herein are useful for the treatment of cancer including solid tumors such as skin, breast, brain, cervical carcinomas, testicular carcinomas, etc. In some embodiments, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above identified conditions.

Testing

For assay of KSP-modulating activity, generally either KSP or a compound according to the invention is non-diffusably bound to an insoluble support having isolated sample receiving areas (e.g., a microtiter plate, an array, etc.). The insoluble support may be made of any composition to which the sample can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are convenient because a large number of assays can be carried out simultaneously, using small amounts of reagents and samples. The particular manner of binding of the sample is not crucial so long as it is compatible with the reagents and overall methods of the invention, maintains the activity of the sample and is nondiffusable. Methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the sample, excess unbound material is removed by washing. The sample receiving areas may then be blocked through incubation with bovine serum albumin (BSA), casein or other innocuous protein or other moiety.

The compounds of the invention may be used on their own to inhibit the activity of a mitotic kinesin, such as KSP. In some embodiments, a compound of the invention is combined with KSP and the activity of KSP is assayed. Kinesin (including KSP) activity is known in the art and includes one or more kinesin activities. Kinesin activities include the ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes, such as kinases or proteases; and specific kinesin cellular activities such as spindle pole separation.

Methods of performing motility assays are well known to those of skill in the art. (See e.g., Hall, et al. (1996), Biophys. J., 71: 3467-3476, Turner et al., 1996, Anal. Biochem. 242 (1): 20-5; Gittes et al., 1996, Biophys. J. 70 (1): 418-29; Shirakawa et al., 1995, J. Exp. Biol. 198: 1809-15; Winkelmann et al., 1995, Biophys. J. 68: 2444-53; Winkelmann et al., 1995, Biophys. J. 68: 72S.)

Methods known in the art for determining ATPase hydrolysis activity also can be used. Generally, solution based assays are utilized. U.S. Pat. No. 6,410,254, hereby incorporated by reference in its entirety, describes such assays. Alternatively, conventional methods are used. For example, Pᵢ release from kinesin can be quantified. In some embodiments, the ATPase hydrolysis activity assay utilizes 0.3 M PCA (perchloric acid) and malachite green reagent (8.27 mM sodium molybdate II, 0.33 mM malachite green oxalate, and 0.8 mM Triton X-1 00). To perform the assay, 10 μL of the reaction mixture is quenched in 90 μL of cold 0.3 M PCA. Phosphate standards are used so data can be converted to mM inorganic phosphate released. When all reactions and standards have been quenched in PCA, 100 μL of malachite green reagent is added to the relevant wells in e.g., a microtiter plate. The mixture is developed for 10-15 minutes and the plate is read at an absorbance of 650 nm. If phosphate standards were used, absorbance readings can be converted to mM Pᵢ and plotted over time. Additionally, ATPase assays known in the art include the luciferase assay.

ATPase activity of kinesin motor domains also can be used to monitor the effects of agents and are well known to those skilled in the art. In some embodiments ATPase assays of kinesin are performed in the absence of microtubules. In some embodiments, the ATPase assays are performed in the presence of microtubules. Different types of agents can be detected in the above assays. In some embodiments, the effect of an agent is independent of the concentration of microtubules and ATP. In some embodiments, the effect of the agents on kinesin ATPase can be decreased by increasing the concentrations of ATP, microtubules or both. In some embodiments, the effect of the agent is increased by increasing concentrations of ATP, microtubules or both.

Compounds that inhibit the biochemical activity of KSP in vitro may then be screened in vivo. In vivo screening methods include assays of cell cycle distribution, cell viability, or the presence, morphology, activity, distribution, or number of mitotic spindles. Methods for monitoring cell cycle distribution of a cell population, for example, by flow cytometry, are well known to those skilled in the art, as are methods for determining cell viability. See for example, U.S. Pat. No. 6,437,115, hereby incorporated by reference in its entirety. Microscopic methods for monitoring spindle formation and malformation are well known to those of skill in the art (see, e.g., Whitehead and Rattner (1998), J. Cell Sci. 111: 2551-61; Galgio et al, (1996) J. Cell Biol., 135: 399-414), each incorporated herein by reference in its entirety.

The compounds of the invention inhibit the KSP kinesin. One measure of inhibition is IC₅₀, defined as the concentration of the compound at which the activity of KSP is decreased by fifty percent relative to a control. Generally, compounds have IC₅₀'s of less than about 1 mM, with some embodiments having IC₅₀'s of less than about 100 μM, with some embodiments having IC₅₀'s of less than about 10 μM, with some embodiments having IC₅₀'s of less than about 1 μM, and with some embodiments having IC₅₀'s of less than about 100 nM, and with some embodiments having IC₅₀'s of less than about 10 nM. Measurement of IC₅₀ is done using an ATPase assay such as described herein.

Another measure of inhibition is Kᵢ. For compounds with IC₅₀'s less than 1 μM, the Kᵢ or K_{d} is defined as the dissociation rate constant for the interaction of the compounds described herein with KSP. Generally, compounds have Kᵢ's of less than about 100 μM, with some embodiments having Kᵢ's of less than about 10 μM, and with some embodiments having Kᵢ's of less than about 1 μM and some embodiments having Kᵢ's of less than about 100 nM, and with some embodiments having Kᵢ's of less than about 10 nM.

The Kᵢ for a compound is determined from the IC₅₀ based on three assumptions and the Michaelis-Menten equation. First, only one compound molecule binds to the enzyme and there is no cooperativity. Second, the concentrations of active enzyme and the compound tested are known (i.e., there are no significant amounts of impurities or inactive forms in the preparations). Third, the enzymatic rate of the enzyme-inhibitor complex is zero. The rate (i.e., compound concentration) data are fitted to the equation:

$$V = {V}_{max} {E}_{0} [ I - \frac{( {E}_{0} + {I}_{0} + Kd ) - \sqrt{{( {E}_{0} + {I}_{0} + Kd )}^{2} - 4 {E}_{0} {I}_{0}}}{2 {E}_{0}} ]$$

where V is the observed rate, Vₘₐₓ is the rate of the free enzyme, 10 is the inhibitor concentration, E₀ is the enzyme concentration, and K_{d} is the dissociation constant of the enzyme-inhibitor complex.

Another measure of inhibition is GI₅₀, defined as the concentration of the compound that results in a decrease in the rate of cell growth by fifty percent. Generally, compounds have GI₅₀'s of less than about 1 mM; those having a GI₅₀ of less than about 20 μM are more preferred; those having a GI₅₀ of less than about 10 μM more so; those having a GI₅₀ of less than about 1 μM more so; those having a GI₅₀ of less than about 100 nM more so; and those having a GI₅₀ of less than about 10 μM even more so. Measurement of GI₅₀ is done using a cell proliferation assay such as described herein. Compounds of this class were found to inhibit cell proliferation.

In vitro potency of small molecule inhibitors is determined, for example, by assaying human ovarian cancer cells (SKOV3) for viability following a 72-hour exposure to a 9-point dilution series of compound. Cell viability is determined by measuring the absorbance of formazon, a product formed by the bioreduction of MTS/PMS, a commercially available reagent. Each point on the dose-response curve is calculated as a percent of untreated control cells at 72 hours minus background absorption (complete cell kill).

Anti-proliferative compounds that have been successfully applied in the clinic to treatment of cancer (cancer chemotherapeutics) have GI₅₀'S that vary greatly. For example, in A549 cells, paclitaxel GI₅₀ is 4 nM, doxorubicin is 63 nM, 5-fluorouracil is 1 μM, and hydroxyurea is 500 μM (data provided by National Cancer Institute, Developmental Therapeutic Program, http://dtp.nci.nih.gov/). Therefore, compounds that inhibit cellular proliferation, irrespective of the concentration demonstrating inhibition, may be useful.

To employ the compounds of the invention in a method of screening for compounds that bind to KSP kinesin, the KSP is bound to a support, and a compound of the invention is added to the assay. Alternatively, the compound of the invention is bound to the support and KSP is added. Classes of compounds among which novel binding agents may be sought include specific antibodies, non-natural binding agents identified in screens of chemical libraries, peptide analogs, etc. Of particular interest are screening assays for candidate agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the compound of the invention to KSP may be done in a number of ways. In some embodiments, the compound is labeled, for example, with a fluorescent or radioactive moiety, and binding is determined directly. For example, this may be done by attaching all or a portion of KSP to a solid support, adding a labeled test compound (for example a compound of the invention in which at least one atom has been replaced by a detectable isotope), washing off excess reagent, and determining whether the amount of the label is that present on the solid support.

By “labeled” herein is meant that the compound is either directly or indirectly labeled with a label which provides a detectable signal, e.g., radioisotope, fluorescent tag, enzyme, antibodies, particles such as magnetic particles, chemiluminescent tag, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the kinesin proteins may be labeled at tyrosine positions using ¹²⁵I, or with fluorophores. Alternatively, more than one component may be labeled with different labels; using ¹²⁵I for the proteins, for example, and a fluorophor for the antimitotic agents.

The compounds of the invention may also be used as competitors to screen for additional drug candidates. “Candidate agent” or “drug candidate” or grammatical equivalents as used herein describe any molecule, e.g., protein, oligopeptide, small organic molecule, polysaccharide, polynucleotide, etc., to be tested for bioactivity. They may be capable of directly or indirectly altering the cellular proliferation phenotype or the expression of a cellular proliferation sequence, including both nucleic acid sequences and protein sequences. In other cases, alteration of cellular proliferation protein binding and/or activity is screened. Screens of this sort may be performed either in the presence or absence of microtubules. In the case where protein binding or activity is screened, certain embodiments exclude molecules already known to bind to that particular protein, for example, polymer structures such as microtubules, and energy sources such as ATP. Some embodiments of assays herein include candidate agents which do not bind the cellular proliferation protein in its endogenous native state termed herein as “exogenous” agents. In some embodiments, exogenous agents further exclude antibodies to KSP.

Candidate agents can encompass numerous chemical classes, though typically they are small organic compounds having a molecular weight of more than 100 and less than about 2,500 daltons. Candidate agents comprise functional groups necessary for structural interaction with proteins, such as hydrogen bonding and lipophilic binding, and typically include at least an amine, carbonyl, hydroxyl, ether, or carboxyl group. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, and/or amidification to produce structural analogs.

Competitive screening assays may be done by combining KSP and a drug candidate in a first sample. A second sample comprises a compound of the present invention, KSP and a drug candidate. This may be performed in either the presence or absence of microtubules. The binding of the drug candidate is determined for both samples, and a change, or difference in binding between the two samples indicates the presence of a drug candidate capable of binding to KSP and potentially inhibiting its activity. That is, if the binding of the drug candidate is different in the second sample relative to the first sample, the drug candidate is capable of binding to KSP.

In some embodiments, the binding of the candidate agent to KSP is determined through the use of competitive binding assays. In some embodiments, the competitor is a binding moiety known to bind to KSP, such as an antibody, peptide, binding partner, ligand, etc. Under certain circumstances, there may be competitive binding as between the candidate agent and the binding moiety, with the binding moiety displacing the candidate agent.

In some embodiments, the candidate agent is labeled. Either the candidate agent, or the competitor, or both, is added first to KSP for a time sufficient to allow binding, if present. Incubations may be performed at any temperature which facilitates optimal activity, typically between 4 and 40° C.

Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

In some embodiments, the competitor is added first, followed by the candidate agent. Displacement of the competitor is an indication the candidate agent is binding to KSP and thus is capable of binding to, and potentially inhibiting, the activity of KSP. In some embodiments, either component can be labeled. Thus, for example, if the competitor is labeled, the presence of label in the wash solution indicates displacement by the agent. Alternatively, if the candidate agent is labeled, the presence of the label on the support indicates displacement.

In some embodiments, the candidate agent is added first, with incubation and washing, followed by the competitor. The absence of binding by the competitor may indicate the candidate agent is bound to KSP with a higher affinity. Thus, if the candidate agent is labeled, the presence of the label on the support, coupled with a lack of competitor binding, may indicate the candidate agent is capable of binding to KSP.

Inhibition is tested by screening for candidate agents capable of inhibiting the activity of KSP comprising the steps of combining a candidate agent with KSP, as above, and determining an alteration in the biological activity of KSP. Thus, in some embodiments, the candidate agent should both bind to KSP (although this may not be necessary), and alter its biological or biochemical activity as defined herein. The methods include both in vitro screening methods and in vivo screening of cells for alterations in cell cycle distribution, cell viability, or for the presence, morpohology, activity, distribution, or amount of mitotic spindles, as are generally outlined above.

Alternatively, differential screening may be used to identify drug candidates that bind to the native KSP, but cannot bind to modified KSP.

Positive controls and negative controls may be used in the assays. Generally all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g., albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Administration

Accordingly, the compounds of the invention are administered to cells. By “cells” herein is meant any cell in which mitosis or meiosis can be altered. By “administered” herein is meant administration of a therapeutically effective dose of a compound of the invention to a cell either in cell culture or in a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for systemic versus localized delivery, route of administration, age, body weight, general health, sex, diet, time of administration, nature of the formulation, drug interaction, and the precise condition requiring treatment and its severity may be necessary, and will be ascertainable with routine experimentation by those skilled in the art. However, an effective amount of a compound of Formula I, II, or III for the treatment of neoplastic growth (typically by intravenous administration), for example colon or breast carcinoma, will generally be in the range of 0.1 to 100 (including 1 to 100) mg/m² of surface area of the recipient per dose on a once a week to once a month schedule and usually in the range of 2 to 30 mg/m² of surface area of the recipient per dose on a once a week to once a month schedule. An effective amount of a salt, solvate, or solvate of a salt of a compound of Formula I, II, or III may be determined as a proportion of the effective amount of the compound of Formula I, II, or III per se. It is envisaged that similar dosages would be appropriate for treatment of the other conditions referred to herein.

A “patient” for the purposes of the present invention includes both humans and other animals, such as mammals, and other organisms. Thus the methods are applicable to both human therapy and veterinary applications. In some embodiments the patient is a mammal, and in some embodiments the patient is human.

Compounds of the invention having the desired pharmacological activity may be administered, generally as a pharmaceutically acceptable composition comprising an pharmaceutical excipient, to a patient, as described herein. Depending upon the manner of introduction, the compounds may be formulated in a variety of ways as discussed below. The concentration of therapeutically active compound in the formulation may vary from about 0.1-100 wt. %.

The agents may be administered alone or in combination with other treatments, i.e., radiation, or other chemotherapeutic agents such as the taxane class of agents that appear to act on microtubule formation or the camptothecin class of topoisomerase I inhibitors. When used, other chemotherapeutic agents may be administered before, concurrently, or after administration of a compound of the present invention. In one aspect of the invention, a compound of the present invention is co-administered with one or more other chemotherapeutic agents. By “co-administer” it is meant that the present compounds are administered to a patient such that the present compounds as well as the co-administered compound may be found in the patient's bloodstream at the same time, regardless when the compounds are actually administered, including simultaneously.

The administration of the compounds and compositions of the present invention can be done in a variety of ways, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the compound or composition may be directly applied as a solution or spray.

Pharmaceutical dosage forms include a compound of Formula I, II, or III or a pharmaceutically acceptable salt or solvate thereof, and one or more pharmaceutical excipients. As is known in the art, pharmaceutical excipients are secondary ingredients which function to enable or enhance the delivery of a drug or medicine in a variety of dosage forms (e.g.: oral forms such as tablets, capsules, and liquids; topical forms such as dermal, opthalmic, and otic forms; suppositories; injectables; respiratory forms and the like). Pharmaceutical excipients include inert or inactive ingredients, synergists or chemicals that substantively contribute to the medicinal effects of the active ingredient. For example, pharmaceutical excipients may function to improve flow characteristics, product uniformity, stability, taste, or appearance, to ease handling and administration of dose, for convenience of use, or to control bioavailability. While pharmaceutical excipients are commonly described as being inert or inactive, it is appreciated in the art that there is a relationship between the properties of the pharmaceutical excipients and the dosage forms containing them.

Pharmaceutical excipients suitable for use as carriers or diluents are well known in the art, and may be used in a variety of formulations. See, e.g., Remington's Pharmaceutical Sciences, 18th Edition, A. R. Gennaro, Editor, Mack Publishing Company (1990); Remington: The Science and Practice of Pharmacy, 20th Edition, A. R. Gennaro, Editor, Lippincott Williams & Wilkins (2000); Handbook of Pharmaceutical Excipients, 3rd Edition, A. H. Kibbe, Editor, American Pharmaceutical Association, and Pharmaceutical Press (2000); and Handbook of Pharmaceutical Additives, compiled by Michael and Irene Ash, Gower (1995), each of which is incorporated herein by reference for all purposes.

Oral solid dosage forms such as tablets will typically comprise one or more pharmaceutical excipients, which may for example help impart satisfactory processing and compression characteristics, or provide additional desirable physical characteristics to the tablet. Such pharmaceutical excipients may be selected from diluents, binders, glidants, lubricants, disintegrants, colors, flavors, sweetening agents, polymers, waxes or other solubility-retarding materials.

Compositions for intravenous administration will generally comprise intravenous fluids, i.e., sterile solutions of simple chemicals such as sugars, amino acids or electrolytes, which can be easily carried by the circulatory system and assimilated. Such fluids are prepared with water for injection USP.

Fluids used commonly for intravenous (IV) use are disclosed in Remington, the Science and Practice of Pharmacy [full citation previously provided], and include:
alcohol (e.g., in dextrose and water (“D/W”) [e.g., 5% dextrose] or dextrose and water [e.g., 5% dextrose] in normal saline solution (“NSS”); e.g. 5% alcohol);
synthetic amino acid such as Aminosyn, FreAmine, Travasol, e.g., 3.5 or 7; 8.5; 3.5, 5.5 or 8.5% respectively;
ammonium chloride e.g., 2.14%;
dextran 40, in NSS e.g., 10% or in D5/W e.g., 10%;
dextran 70, in NSS e.g., 6% or in D5/W e.g., 6%;
dextrose (glucose, D5/W) e.g., 2.5-50%;
dextrose and sodium chloride e.g., 5-20% dextrose and 0.22-0.9% NaCl;
lactated Ringer's (Hartmann's) e.g., NaCl 0.6%, KCl 0.03%, CaCl₂ 0.02%;
lactate 0.3%;
mannitol e.g., 5%, optionally in combination with dextrose e.g., 10% or NaCl e.g., 15 or 20%;
multiple electrolyte solutions with varying combinations of electrolytes, dextrose, fructose, invert sugar Ringer's e.g., NaCl 0.86%, KCl 0.03%, CaCl₂ 0.033%;
sodium bicarbonate e.g., 5%;
sodium chloride e.g., 0.45, 0.9, 3, or 5%;
sodium lactate e.g., ⅙ M; and
sterile water for injection
   The pH of such fluids may vary, and will typically be from 3.5 to 8 such as known in the art.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All publications, including but not limited to patents and patent applications, cited in this specification are herein incorporated by reference as if each individual publication were specifically and individually indicated to be incorporated by reference herein as though fully set forth.

### EXAMPLES

All anhydrous solvents were purchased from Aldrich Chemical Company in SureSeal® containers. Reagents were added and aqueous extractions performed with single or multichannel pipettors. Filtrations were performed using Whatman/Polyfiltronics 24 well, 10 mL filtration blocks. Evaporation of volatile materials from the array was performed with a Labconco Vortex-Evaporator or by sweeping with a 4×6 nitrogen manifold.

### Example 1

Synthesis of Compounds 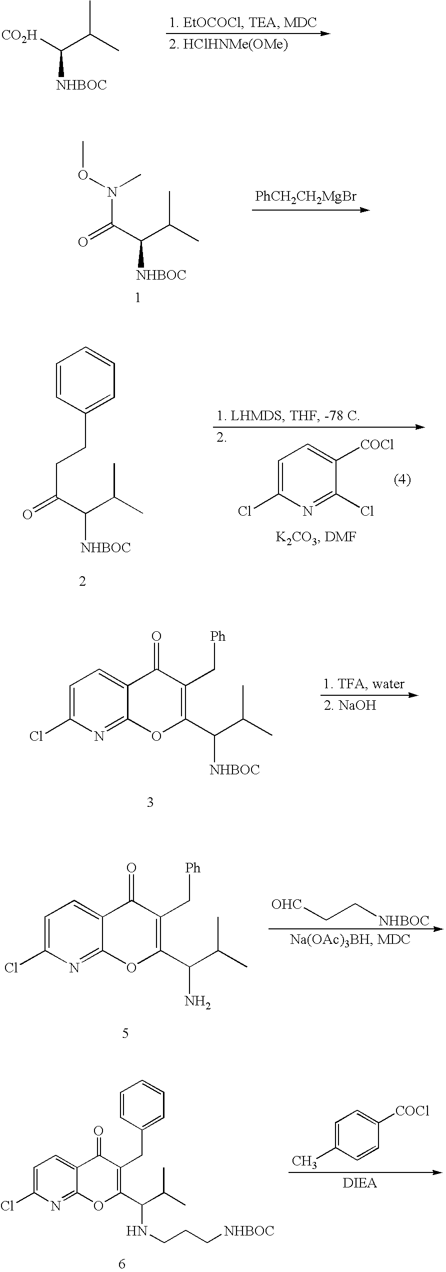 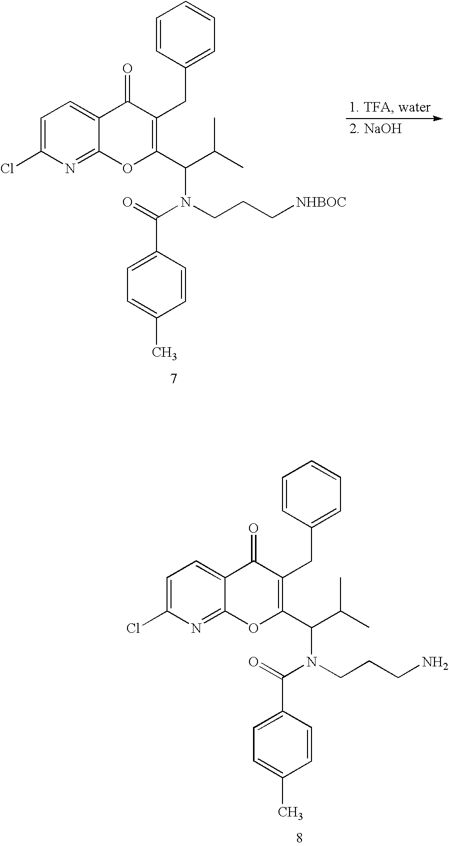
a). N²-{[(1,1-Dimethylethyl)oxy]carbonyl}-N¹-methyl-N¹-(methyloxy)valinamide (1)

Ethylchloroformate (11.0 mL, 115 mmol) was added over 1 minute to a 0-5° C. solution of BOC-D-Valine (25.0 g, 115 mmol), triethylamine (16.0 mL, 115 mmol), and THF (145 mL) under N₂. The internal temperature of the reaction solution rose to 9° C. After 15 mins, a mixture of dimethylhydroxylamine hydrochloride (13.46 g, 138 mmol), triethylamine (32.0 mL, 230 mmol), and THF (110 mL) was added over 5 minutes. The internal temperature rose to 17° C. Upon completion of addition, the ice/H₂O bath was removed and the reaction solution maintained at 23° C. for 1 hour. The reaction solution was then concentrated. The crude residue was dissolved in EtOAc (200 mL) and washed with 1 N HCl (200 mL) and brine (100 mL). The organic layer was dried (MgSO₄), filtered, and concentrated to provide 30 g (˜100%) of 1 as a colorless oil, which was used without further purification.
b). 1,1-Dimethylethyl [1-(1-methylethyl)-2-oxo-4-phenylbutyl]carbamate (2).

(2-Bromoethyl)benzene (38.0 mL, 273 mmol), magnesium turnings (7.0 g, 289 mmol), and Et₂O (500 mL) were mixed in a 1 L round-bottom flask equipped with a reflux condenser at 23° C. under a N₂ atmosphere. After ˜10 mins the reaction mixture begins to exotherm and the reaction mixture was allowed to progress to reflux with intermittent cooling with an ice/H₂O bath. After 1.5 hour, the Grignard reaction was complete and the solution had cooled to 23° C. A solution of 1 (18.0 g, 82.7 mmol) and Et₂O (200 mL) was added via cannula to the 20° C. solution of the phenethylmagnesium bromide. The temperature was monitored by internal thermometer and was not allowed to exceed ˜30° C. The reaction mixture temperature was monitored by an internal thermometer and regulated (20-30° C.) with an ice/H₂O bath. After 1 h at 23° C., the reaction mixture was quenched by pouring into 1 N HCl (300 mL). The layers were separated and the organic layer was washed with brine (100 mL). The organic layer was dried (MgSO₄), filtered, and concentrated. The resulting residue was purified by flash column chromatography (10:1 hexanes:EtOAc) to yield 13.4 g (53%) of 2. LRMS (MH-tBuOCO) m/z 206.1.
c) 1,1-Dimethylethyl {1-[7-chloro-4-oxo-3-(phenylmethyl)-4H-pyrano[2,3-b]pyridin-2-yl]-2-methylpropyl} carbamate (3).

Lithium bis(trimethylsilyl)amide (LHMDS, 1.0 M in THF, 94.0 mL, 3.3 equiv) was added slowly over ˜3 minutes via syringe to a −78° C. solution of ketone 2 (8.74 g, 28.62 mmol) and THF (100 mL). The reaction solution temperature was monitored by an internal thermometer, and addition of the base was done at a rate sufficient to prevent the temperature from exceeding −54° C. After the addition was complete the resulting solution was maintained at −78° C. for 30 mins. Neat 2,6-dichloronicotinoyl chloride (U.S. Pat. No. 6,511,974, 4, 5.9 g, 28 mmol) was added drop-wise over ˜1 minute via syringe. The reaction solution was maintained at −78° C. for 30 mins. The reaction solution was quenched with saturated aqueous ammonium chloride (100 mL). The layers were separated and the organic layer was washed with brine (100 mL). The organic layer was dried (MgSO₄), filtered, and concentrated. The resulting residue, used without further purification, was treated with K₂CO₃ (excess), and DMF (12 mL) at r.t. for 30 mins. The reaction was quenched with brine (50 mL), and diluted with Et₂O (50 mL). The layers were separated and the organic layer was washed with brine (2×50 mL). The organic layer was dried (MgSO₄), filtered, and concentrated. The resulting residue was purified by flash column chromatography.
d). 2-(1-Amino-2-methylpropyl)-7-chloro-3-(phenylmethyl)-4H-pyrano[2,3-b]pyridin-4-one (5).

Chromenone 3 (4.18 mmol) and TFA:H₂O (97.5:2.5, 30 mL) was maintained at 23° C. for 1 h. The reaction mixture was concentrated. The residue was dissolved in EtOAc (100 mL) and washed with 1 N NaOH (25 mL) and brine (25 mL). The organic layer was dried (MgSO₄), filtered, and concentrated to provide 5 which was used without further purification.
e). 1,1-Dimethylethyl [3-({1-[7-chloro-4-oxo-3-(phenylmethyl)-4H-pyrano[2,3-b]pyridin-2-yl]-2-methylpropyl}amino)propyl]carbamate (6).

Chromenone 5 (0.22 mmol), 1,1-dimethylethyl (3-oxopropyl)carbamate (0.32 mmol), Na(OAc)₃BH (0.87 mmol), and CH₂Cl₂ (1 mL) was maintained at 23° C. for 3 h. The reaction mixture was diluted with EtOAc (20 mL) and washed with 1 N NaOH (5 mL) and brine (5 mL). The organic layer was dried (MgSO₄), filtered, and concentrated. The resulting residue was purified by flash column chromatography to yield 6.
f). 1,1-Dimethylethyl (3-{{1-[7-chloro-4-oxo-3-(phenylmethyl)-4H-pyrano[2,3-b]pyridin-2-yl]-2-methylpropyl}[(4-methylphenyl)carbonyl]amino}propyl)carbamate (7).

To a solution of chromenone 6 (2.6 mmol), diisopropylethylamine (DIEA, 1.8 mL), and CH₂Cl₂ (7.5 mL) at 23° C. was added p-toluoyl chloride (5.22 mmol). After 2.5 h, the reaction mixture was diluted with EtOAc (100 mL) and washed with saturated aqueous NaHCO₃ (2×20 mL) and brine (20 mL). The organic layer was dried (MgSO₄), filtered, and concentrated. The resulting residue was purified by flash column chromatography to yield 7 as a colorless oil.
g). N-(3-aminopropyl)-N-{1-[7-chloro-4-oxo-3-(phenylmethyl)-4H-pyrano[2,3-b]pyridin-2-yl]-2-methylpropyl}-4-methylbenzamide (8).

Chromenone 7 (2.32 mmol) and TFA:H₂O (97.5:2.5, 30 mL) was maintained at 23° C. for 1 h. The reaction mixture was concentrated. The residue was dissolved in EtOAc (100 mL) and washed with 1 N NaOH (25 mL) and brine (25 mL). The organic layer was dried (MgSO₄), filtered, and concentrated to provide the title compound.

### Example 2

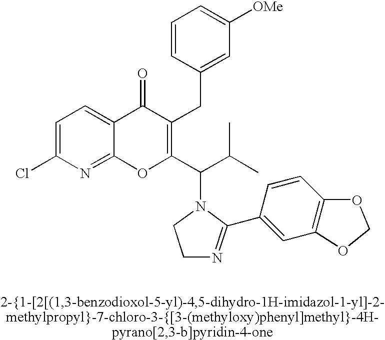 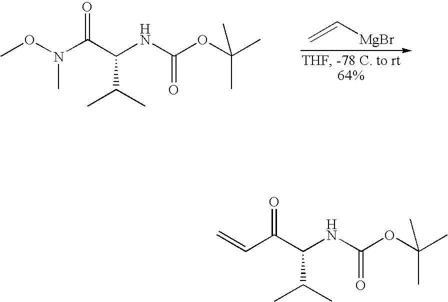 [0360] a). ((R)-1-Isopropyl-2-oxo-but-3-enyl)-carbamic Acid tert-butyl Ester

Tetrahydrofuran (THF, 100 mL) and a 1.0M solution of vinyl magnesium bromide in THF (360 mL, 360 mmol, 3.1 equiv) was cooled to −78° C. while stirring under a nitrogen atmosphere. The mixture was treated dropwise with a solution of [(R)-(methoxy-methyl-carbamoyl)-methyl-propyl]-carbamic acid tert-butyl ester (30.3 g, 116 mmol, 1 equiv) in THF (50 mL) over 30 min. After the resultant dark yellow mixture was stirred for 30 min at −78° C., the cooling bath was removed and the reaction mixture was warmed slowly to room temperature overnight (15 h). The reaction mixture was poured slowly into an ice-chilled solution of 1N aqueous hydrochloric acid (700 mL) and then warmed to room temperature. The organics were extracted with ethyl acetate (3×600 mL), dried over sodium sulfate, filtered, and concentrated in vacuo. Purification by flash column chromatography (5-10% ethyl acetate/hexanes) provided the product as a white solid (16.8 g, 64%). ESMS [M+H]⁺: 228.4. 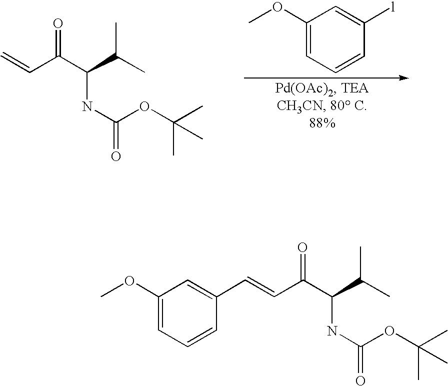
b). [(R)-(E)-1-Isopropyl-4-(3-methoxy-phenyl)-2-oxo-but-3-enyl]-carbamic Acid tert-butyl Ester

To a solution of ((R)-1-Isopropyl-2-oxo-but-3-enyl)-carbamic acid tert-butyl ester (13.54 g, 59.6 mmol) in dry acetonitrile (150 mL) under argon, was added 3-iodoanisole (13.96 g, 59.6 mmol), triethylamine (9.1 mL, 65.6 mmol) followed by palladium (II) acetate (335 mg, 1.49 mmol). The resulting clear yellow solution was heated to 80° C. Upon heating, the reaction darkened and the precipitation of palladium black occurred. After 15 h, the reaction mixture was allowed to cool to room temperature, quenched with water (150 mL) and diluted with ether (150 mL). The ether layer was washed with brine (100 mL) and the combined aqueous layers were extracted with ether (two 50 mL portions). The extracts were dried over magnesium sulfate, filtered and concentrated under reduced pressure. The residue was immediately purified by silica gel chromatography (9:1 hexanes/EtOAc) to provide 17.6 g (88%) of [(R)-(E)-1-isopropyl-4-(3-methoxy-phenyl)-2-oxo-but-3-enyl]-carbamic acid tert-butyl ester as a yellow oil. MS(ES+) m/e 334.0 [M+H]⁺. 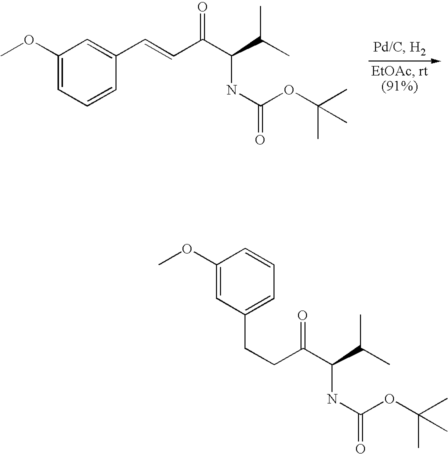
c). [(R)-(E)-1-Isopropyl-4-(3-methoxy-phenyl)-2-oxo-butyl]-carbamic acid tert-butyl ester

To a solution of [(R)-(E)-1-isopropyl-4-(3-methoxy-phenyl)-2-oxo-but-3-enyl]-carbamic acid tert-butyl ester (17.6 g, 52.9 mmol) in ethyl acetate (450 mL) under nitrogen was added 10 wt % palladium on carbon (300 mg). The nitrogen was replaced with a balloon of hydrogen and the flask was purged. After 3 h, the reaction flask was purged with nitrogen and filtered through a pad of celite (rinsing with ethyl acetate). The filtrate was concentrated under reduced pressure and the residue was purified by silica gel chromatography (9:1 hexanes/EtOAc) to provide 16.2 g (91%) of [(R)-(E)-1-isopropyl-4-(3-methoxy-phenyl)-2-oxo-butyl]-carbamic acid tert-butyl ester as a colorless oil. MS(ES+) m/e 336.4 [M+H]⁺. [α]_{D}²⁰=+19.1 (c=0.755, MeOH). ¹H NMR (400 MHz, CDCl₃) δ 7.21 (m, 1H), 6.80-6.77 (m, 2H), 6.75 (s, 1H), 5.13 (d, J=8.4 Hz, 1H), 4.28 (dd, J=8.8, 4.4 Hz, 1H), 3.81 (s, 3H), 2.93-2.88 (m, 2H), 2.85-2.76 (m, 2H), 2.14 (m, 1H), 1.46 (s, 9H), 1.00 (d, J=6.8 Hz, 3H), 0.75 (d, J=6.8 Hz, 3H).
d). 1,1-Dimethylethyl [1-(7-chloro-3-{[3-(methyloxy)phenyl]methyl}-4-oxo-4H-pyrano[2,3-b]pyridin-2-yl)-2-methylpropyl]carbamate

Substituting [(R)-(E)-1-isopropyl-4-(3-methoxy-phenyl)-2-oxo-butyl]-carbamic acid tert-butyl ester for 1,1-dimethylethyl [1-(1-methylethyl)-2-oxo-4-phenylbutyl]carbamate and using the appropriate molar equivalents of the other reagents and the procedure of Example 1(c) above gave 1,1-dimethylethyl [1-(7-chloro-3-{[3-(methyloxy)phenyl]methyl}-4-oxo-4H-pyrano[2,3-b]pyridin-2-yl)-2-methylpropyl]carbamate.
e). 2-(1-Amino-2-methylpropyl)-7-chloro-3-{[3-(methyloxy)phenyl]methyl}-4H-pyrano[2,3-b]pyridin-4-one.

Substituting 1,1-dimethylethyl [1-(7-chloro-3-{[3-(methyloxy)phenyl]methyl}-4-oxo-4H-pyrano[2,3-b]pyridin-2-yl)-2-methylpropyl]carbamate for 1,1-dimethylethyl {1-[7-chloro-4-oxo-3-(phenylmethyl)-4H-pyrano[2,3-b]pyridin-2-yl]-2-methylpropyl} carbamate and using the appropriate molar equivalents of the other reagents and the procedure of Example 1 (d) above gave 2-(1-amino-2-methylpropyl)-7-chloro-3-{[3-(methyloxy)phenyl]methyl}-4H-pyrano[2,3-b]pyridin-4-one.
f). 1,1-Dimethylethyl (2-{[1-(7-chloro-3-{[3-(methyloxy)phenyl]methyl}-4-oxo-4H-pyrano[2,3-b]pyridin-2-yl)-2-methylpropyl]amino} ethyl)carbamate

To a solution 2-(1-amino-2-methylpropyl)-7-chloro-3-{[3-(methyloxy)phenyl]methyl}-4H-pyrano[2,3-b]pyridin-4-one. (18 mmol) and (2-oxoethyl)carbamic acid tert-butyl ester (23 mmol) in methylene chloride (150 mL) was added sodium triacetoxyborohydride (35 mmol). The reaction mixture was stirred at room temperature for 16 hours, at which time it was diluted with 1 N sodium hydroxide (150 mL) and stirred vigorously for 2 hours. The organic layer was washed with 1 N sodium hydroxide (100 mL) and brine (100 mL), dried over magnesium sulfate, and concentrated. The residue was purified by flash chromatography to give 1,1-Dimethylethyl (2-{[1-(7-chloro-3-{[3-(methyloxy)phenyl]methyl}-4-oxo-4H-pyrano[2,3-b]pyridin-2-yl)-2-methylpropyl]amino}ethyl)carbamate.
g) 2-{1-[(2-Aminoethyl)amino]-2-methylpropyl}-7-chloro-3-{[3-(methyloxy)phenyl]methyl}-4H-pyrano[2,3-b]pyridin-4-one

A solution of 1,1-dimethylethyl (2-{[1-(7-chloro-3-{[3-(methyloxy)phenyl]methyl}-4-oxo-4H-pyrano[2,3-b]pyridin-2-yl)-2-methylpropyl]amino} ethyl)carbamate (12 mmol) in 4:1 methylene chloride/trifluoroacetic acid (250 mL) was maintained at room temperature for 1.5 hours, at which time it was concentrated. The residue was dissolved in methylene chloride (200 mL), washed with 10% sodium carbonate, saturated sodium bicarbonate, and brine, dried over magnesium sulfate, and concentrated. The residue was used without futher puification.
h) N-(2-{[1-(7-Chloro-3-{[3-(methyloxy)phenyl]methyl}-4-oxo-4H-pyrano [2,3-b]pyridin-2-yl)-2-methylpropyl]amino}ethyl)-1,3-benzodioxole-5-carboxamide

To a cooled (0° C.) solution of 2-{1-[(2-aminoethyl)amino]-2-methylpropyl}-7-chloro-3-{[3-(methyloxy)phenyl]methyl}-4H-pyrano[2,3-b]pyridin-4-one (11 mmol) and triethylamine (17 mmol) in methylene chloride (100 mL) was added a solution of piperonyl chloride (11 mmol) in methylene chloride (20 mL). The reaction was maintained at 0° C. for 2 hours, at which time it was diluted with ether (250 mL). The resultant solution was washed with 1 N hydrogen chloride (2×200 mL), saturated sodium bicarbonate (200 mL) and brine (150 mL), dried over magnesium sulfate, and concentrated. The residue was purified by flash chromatography to give the title compound.
i) 2-{1-[2-(1,3-benzodioxol-5-yl)-4,5-dihydro-1H-imidazol-1-yl]-2-methylpropyl}-7-chloro-3-{[3-(methyloxy)phenyl]methyl}-4H-pyrano[2,3-b]pyridin-4-one

A mixture of N-(2-{[1-(7-chloro-3-{[3-(methyloxy)phenyl]methyl}-4-oxo-4H-pyrano[2,3-b]pyridin-2-yl)-2-methylpropyl]amino}ethyl)-1,3-benzodioxole-5-carboxamide (5.5 mmol) and phosphorus oxychloride (100 mmol) in toluene (60 mL) was heated at 85° C. for 7 hours, then heated at reflux for 1 hour. The reaction was concentrated and the residual phosphorus oxychloride removed by toluene azeotrope. The residue was diluted with ethyl acetate (100 mL) and washed with saturated sodium bicarbonate (100 mL) and brine (100 mL), dried over magnesium sulfate, and concentrated. The resultant residue was purified by flash to give the title compound.

### Example 3

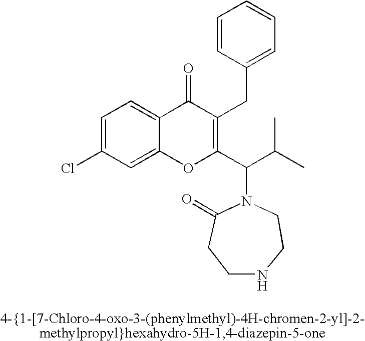 [0379] a) (2-Oxo-ethyl)-carbamic Acid tert-butyl Ester.

To a stirred solution of oxalyl chloride (1.92 mL, 22 mmol) in CH₂Cl₂ (40 mL) was added dropwise DMSO (3.12 mL, 44 mmol) at −78° C. After 15 min., a solution of (2-hydroxy-ethyl)-carbamic acid tert-butyl ester (3.22 g, 20 mmol) in CH₂Cl₂ (20 mL) was added. After another 45 min., Et₃N (13.9 mL, 100 mmol) was added. The reaction mixture was then warmed to room temperature, diluted with CH₂Cl₂ (100 mL), washed with water, 10% HCl, brine, dried and concentrated. Purification by flash chromatography on silica gel (10-15% EtOAc in hexane) gave the title compound (600 mg) as a clear oil: ¹H NMR (400 MHz, CDCl₃) δ 9.58 (S, 1H), 5.16 (br s, 1H), 4.02 (s, 2H), 1.46 (s, 9H).
b) 1,1-Dimethylethyl [2-({1-[7-chloro-4-oxo-3-(phenylmethyl)-4H-chromen-2-yl]-2-methylpropyl}amino)ethyl]carbamate

Sodium triacetoxyborohydride (0.81 mmol) was added to a solution containing 2-(1-amino-2-methylpropyl)-7-chloro-3-(phenylmethyl)-4H-chromen-4-one (0.54 mmol) and (2-oxo-ethyl)-carbamic acid tert-butyl ester (0.65 mmol) in CH₂Cl₂ (5.4 mL). The resulting mixture was stirred at room temperature overnight. The reaction was diluted with CH₂Cl₂ (10 mL), washed with brine, dried and concentrated under vacuum. Purification by flash chromatography on silica gel gave the title compound.
c) 1,1-Dimethylethyl [2-(acryloyl {1-[7-chloro-4-oxo-3-(phenylmethyl)-4H-chromen-2-yl]-2-methylpropyl}amino)ethyl]carbamate

Acryloyl chloride (1.6 mmol) was added to 1,1-dimethylethyl [2-({1-[7-chloro-4-oxo-3-(phenylmethyl)-4H-chromen-2-yl]-2-methylpropyl} amino)ethyl]carbamate (1.03 mmol) and Et₃N (0.15 mmol) in CH₂Cl₂ (10 mL). The resulting mixture was stirred at room temperature overnight. The reaction mixture was diluted with CH₂Cl₂ (10 mL), washed with brine, dried and concentrated under vacuum. Purification by flash chromatography on silica gel gave the title compound.
d) N-(2-aminoethyl)-N-{1-[7-chloro-4-oxo-3-(phenylmethyl)-4H-chromen-2-yl]-2-methylpropyl}-2-propenamide

1,1-Dimethylethyl [2-(acryloyl {1-[7-chloro-4-oxo-3-(phenylmethyl)-4H-chromen-2-yl]-2-methylpropyl}amino)ethyl]carbamate (0.56 mmol) was treated with 50% TFA in CH₂Cl₂ (3 mL) at room temperature. After 2 h the mixture was concentrated under vacuum, redissolved in CH₂Cl₂, washed with 10% NaHCO₃, brine, dried and concentrated to give the title compound.
e) 4-{1-[7-Chloro-4-oxo-3-(phenylmethyl)-4H-chromen-2-yl]-2-methylpropyl}hexahydro-5H-1,4-diazepin-5-one

A solution N-(2-aminoethyl)-N-{1-[7-chloro-4-oxo-3-(phenylmethyl)-4H-chromen-2-yl]-2-methylpropyl}-2-propenamide (0.53 mmol) in MeOH was refluxed under argon overnight. The reaction mixture was concentrated and the residue purified by flash chromatography on silica gel to give the title compound.

### Example 4

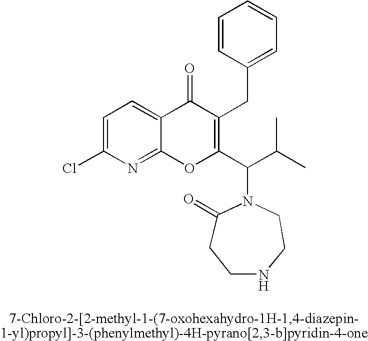 [0390] a) 1,1-Dimethylethyl [2-({1-[7-chloro-4-oxo-3-(phenylmethyl)-4H-pyrano[2,3-b]pyridin-2-yl]-2-methylpropyl} amino)ethyl]carbamate

Sodium triacetoxyborohydride (0.81 mmol) was added to a solution containing 2-(1-amino-2-methylpropyl)-7-chloro-3-(phenylmethyl)-4H-pyrano[2,3-b]pyridin-4-one (0.54 mmol, prepared according to the procedure of Example 1d above) and (2-oxo-ethyl)-carbamic acid tert-butyl ester (0.65 mmol) in CH₂Cl₂ (5 mL). The resulting mixture was stirred at room temperature overnight. The reaction was diluted with CH₂Cl₂, washed with brine, dried and concentrated under vacuum. Purification by flash chromatography on silica gel gave the title compound.
b) 1,1-Dimethylethyl [2-(acryloyl{1-[7-chloro-4-oxo-3-(phenylmethyl)-4H-pyrano[2,3-b]pyridin-2-yl]-2-methylpropyl}amino)ethyl]carbamate

Acryloyl chloride (1.6 mmol) was added to 1,1-dimethylethyl [2-({1-[7-chloro-4-oxo-3-(phenylmethyl)-4H-pyrano[2,3-b]pyridin-2-yl]-2-methylpropyl} amino)ethyl]carbamate (1 mmol) and Et₃N (0.15 mmol) in CH₂Cl₂ (10 mL). The resulting mixture was stirred at room temperature overnight. The reaction mixture was diluted with CH₂Cl₂ (10 mL), washed with brine, dried and concentrated under vacuum. Purification by flash chromatography on silica gel gave the title compound.
c) N-(2-Aminoethyl)-N-{1-[7-chloro-4-oxo-3-(phenylmethyl)-4H-pyrano[2,3-b]pyridin-2-yl]-2-methylpropyl}-2-propenamide

1,1-Dimethylethyl [2-(acryloyl{1-[7-chloro-4-oxo-3-(phenylmethyl)-4H-pyrano[2,3-b]pyridin-2-yl]-2-methylpropyl}amino)ethyl]carbamate (0.56 mmol) was treated with 50% TFA in CH₂Cl₂ (3 mL) at room temperature. After 2 h the mixture was concentrated under vacuum, redissolved in CH₂Cl₂, washed with 10% NaHCO₃, brine, dried and concentrated to give the title compound.
d) 7-Chloro-2-[2-methyl-1-(7-oxohexahydro-1H-1,4-diazepin-1-yl)propyl]-3-(phenylmethyl)-4H-pyrano[2,3-b]pyridin-4-one

A solution N-(2-aminoethyl)-N-{1-[7-chloro-4-oxo-3-(phenylmethyl)-4H-pyrano[2,3-b]pyridin-2-yl]-2-methylpropyl}-2-propenamide (0.53 mmol) in MeOH was refluxed under argon overnight. The reaction mixture was concentrated and the residue purified by flash chromatography on silica gel to give the title compound.

### Example 5

Inhibition of Cellular Viability in Tumor Cell Lines Treated with KSP Inhibitors Materials and Solutions:
Cells: SKOV3, Ovarian Cancer (human).
Media: Phenol Red Free RPMI+5% Fetal Bovine Serum+2 mM L-glutamine.
Colorimetric Agent for Determining Cell Viability: Promega MTS tetrazolium compound.
Control Compound for max cell kill: Topotecan, 1 μM.
   Procedure: Day 1—Cell Plating:

Adherent SKOV3 cells are washed with 10 mLs of PBS followed by the addition of 2 mLs of 0.25% trypsin and incubation for 5 minutes at 37° C. The cells are rinsed from the flask using 8 mL of media (phenol red-free RPMI+5% FBS) and transferred to fresh flask. Cell concentration is determined using a Coulter counter and the appropriate volume of cells to achieve 1000 cells/100 μL is calculated. 100 μL of media cell suspension (adjusted to 1000 cells/100 μL) is added to all wells of 96-well plates, followed by incubation for 18 to 24 hours at 37° C., 100% humidity, and 5% CO₂, allowing the cells to adhere to the plates.

Procedure: Day 2—Compound Addition:

To one column of the wells of an autoclaved assay block are added an initial 2.5 μL of test compound(s) at 400× the highest desired concentration. 1.25 μL of 400× (400 μM) Topotecan is added to other wells (optical density's from these wells are used to subtract out for background absorbance of dead cells and vehicle). 500 μL of media without DMSO are added to the wells containing test compound, and 250 μL to the Topotecan wells. 250 μL of media+0.5% DMSO is added to all remaining wells, into which the test compound(s) are serially diluted. By row, compound-containing media is replica plated (in duplicate) from the assay block to the corresponding cell plates. The cell plates are incubated for 72 hours at 37° C., 100% humidity, and 5% CO₂.

Procedure: Day 4—MTS Addition and OD Reading:

The plates are removed from the incubator and 40 μl MTS/PMS is added to each well. Plates are then incubated for 120 minutes at 37° C., 100% humidity, 5% CO₂, followed by reading the ODs at 490 nm after a 5 second shaking cycle in a ninety-six well spectrophotometer.

Data Analysis

The normalized % of control (absorbance—background) is calculated and an XLfit is used to generate a dose-response curve from which the concentration of compound required to inhibit viability by 50% is determined. The compounds of the present invention show activity when tested by this method as described above.

### Example 6

Monopolar Spindle Formation following Application of a KSP Inhibitor

Human tumor cells Skov-3 (ovarian) were plated in 96-well plates at densities of 4,000 cells per well, allowed to adhere for 24 hours, and treated with various concentrations of the chromenone compounds for 24 hours. Cells were fixed in 4% formaldehyde and stained with antitubulin antibodies (subsequently recognized using fluorescently-labeled secondary antibody) and Hoechst dye (which stains DNA).

Visual inspection revealed that the compounds caused cell cycle arrest in the prometaphase stage of mitosis. DNA was condensed and spindle formation had initiated, but arrested cells uniformly displayed monopolar spindles, indicating that there was an inhibition of spindle pole body separation. Microinjection of anti-KSP antibodies also causes mitotic arrest with arrested cells displaying monopolar spindles.

### Example 7

Inhibition of Cellular Proliferation in Tumor Cell Lines Treated with KSP Inhibitors

Cells were plated in 96-well plates at densities from 1000-2500 cells/well of a 96-well plate and allowed to adhere/grow for 24 hours. They were then treated with various concentrations of drug for 48 hours. The time at which compounds are added is considered T₀. A tetrazolium-based assay using the reagent 3-(4,5-dimethylthiazol-2-yl)-5-(3-carboxymethoxyphenyl)-2-(4-sulfophenyl)-2H-tetrazolium (MTS) (Pat. No. 5,185,450) (see Promega product catalog #G3580, CellTiter 96® AQᵤᵣₑₒᵤₛ One Solution Cell Proliferation Assay) was used to determine the number of viable cells at T₀ and the number of cells remaining after 48 hours compound exposure. The number of cells remaining after 48 hours was compared to the number of viable cells at the time of drug addition, allowing for calculation of growth inhibition.

The growth over 48 hours of cells in control wells that had been treated with vehicle only (0.25% DMSO) is considered 100% growth and the growth of cells in wells with compounds is compared to this.

A GI₅₀ was calculated by plotting the concentration of compound in μM vs the percentage of cell growth in treated wells. The Gi₅₀ calculated for the compounds is the estimated concentration at which growth is inhibited by 50% compared to control, i.e., the concentration at which:
100×[(Treated₄₈−T₀)/(Control₄₈−T₀)]=50
wherein Treated₄₈ is the value at 48 hours for the treated cells and Control₄₈ is the value at 48 hours for the control population.

All concentrations of compounds are tested in duplicate and controls are averaged over 12 wells. A very similar 96-well plate layout and Gi₅₀ calculation scheme is used by the National Cancer Institute (see Monks, et al., J. Natl. Cancer Inst. 83: 757-766 (1991)). However, the method by which the National Cancer Institute quantitates cell number does not use MTS, but instead employs alternative methods.

Compounds of Examples 1-4 above inhibited cell proliferation in human ovarian tumor cell lines (SKOV-3).

### Example 8

Calculation of IC₅₀:

Measurement of a compound's IC₅₀ for KSP activity uses an ATPase assay. The following solutions are used: Solution 1 consists of 3 mM phosphoenolpyruvate potassium salt (Sigma P-7127), 2 mM ATP (Sigma A-3377), 1 mM IDTT (Sigma D-9779), 5 μM paclitaxel (Sigma T-7402), 10 ppm antifoam 289 (Sigma A-8436), 25 mM Pipes/KOH pH 6.8 (Sigma P6757), 2 mM MgCl2 (VWR JT400301), and 1 mM EGTA (Sigma E3889). Solution 2 consists of 1 mM NADH (Sigma N8129), 0.2 mg/ml BSA (Sigma A7906), pyruvate kinase 7 U/ml, L-lactate dehydrogenase 10 U/ml (Sigma PO₂₉₄), 100 nM KSP motor domain, 50 μg/ml microtubules, 1 mM DTT (Sigma D9779), 5 μM paclitaxel (Sigma T-7402), 10 ppm antifoam 289 (Sigma A-8436), 25 mM Pipes/KOH pH 6.8 (Sigma P6757), 2 mM MgCl2 (VWR JT4003-01), and 1 mM EGTA (Sigma E3889). Serial dilutions (8-12 two-fold dilutions) of the compound are made in a 96-well microtiter plate (Corning Costar 3695) using Solution 1. Following serial dilution each well has 50 μl of Solution 1. The reaction is started by adding 50 μl of solution 2 to each well. This may be done with a multichannel pipettor either manually or with automated liquid handling devices. The microtiter plate is then transferred to a microplate absorbance reader and multiple absorbance readings at 340 nm are taken for each well in a kinetic mode. The observed rate of change, which is proportional to the ATPase rate, is then plotted as a function of the compound concentration. For a standard IC₅₀ determination the data acquired is fit by the following four parameter equation using a nonlinear fitting program (e.g., Grafit 4):

$$y = \frac{Range}{1 + {( \frac{x}{{IC}_{50}} )}^{s}} + \text{Background}$$

where y is the observed rate and x is the compound concentration.

1. A compound having the structure: 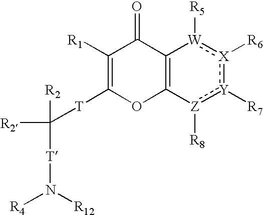
wherein:
W, X, Y, and Z are independently N, C, O, or S, and Z is optionally absent, provided that:
the ring comprising W, X, Y, and optionally Z is heteroaromatic;
at least one of W, X, Y, and Z is not C;
no more than two of W, X, Y, and Z is —N═, and
W, X, or Y is O or S only when Z is absent;
the dashed lines in the structure depict optional double bonds;
T and T′ are independently a covalent bond or optionally substituted lower alkylene;
R₁ is chosen from hydrogen, optionally substituted alkyl-, optionally substituted aryl-, optionally substituted aralkyl-, optionally substituted heteroaryl-, and optionally substituted heteroaralkyl-;
R₂ and R₂ are independently chosen from hydrogen, optionally substituted alkyl-, optionally substituted aryl-, optionally substituted aralkyl-, optionally substituted heteroaryl-, and optionally substituted heteroaralkyl-; or R₂ and R₂ taken together form an optionally substituted 3- to 7-membered ring that optionally incorporates in the ring between zero and two heteroatoms selected from N, O, and S;
R₁₂ is chosen from hydrogen, optionally substituted alkyl-, optionally substituted aryl-, optionally substituted aralkyl-, optionally substituted heteroaryl-, optionally substituted heteroaralkyl-, —C(O)—R₃, and —S(O)₂—R₃ₐ;
R₄ is chosen from hydrogen, optionally substituted alkyl-, optionally substituted aryl-, optionally substituted aralkyl-, optionally substituted heteroaralkyl-, and optionally substituted heterocyclyl-;
or R₄ taken together with R₁₂ and the nitrogen to which they are bound, form an optionally substituted 5- to 12-membered nitrogen-containing heterocycle, which optionally incorporates from one to two additional heteroatoms, selected from N, O, and S, in the heterocycle ring;
or R₄ taken together with R₂ form an optionally substituted 5- to 12-membered nitrogen-containing heterocycle, which optionally incorporates from one to two additional heteroatoms, selected from N, O, and S in the heterocycle ring;
R₃ is chosen from hydrogen, optionally substituted alkyl-, optionally substituted aryl-, optionally substituted aralkyl-, optionally substituted heteroaryl-, optionally substituted heteroaralkyl-, R₁₅O— and R₁₇—NH—;
R₃ₐ is chosen from optionally substituted alkyl-, optionally substituted aryl-, optionally substituted aralkyl-, optionally substituted heteroaryl-, optionally substituted heteroaralkyl-, and R₁₇—NH—;
R₅, R₆, R₇ and R₈ are independently chosen from hydrogen, acyl, optionally substituted alkyl-, optionally substituted alkoxy, halogen, hydroxyl, nitro, cyano, optionally substituted amino, alkylsulfonyl-, alkylsulfonamido-, alkylthio-, carboxyalkyl-, carboxamido-, aminocarbonyl-, optionally substituted aryl and optionally substituted heteroaryl-, provided that R₅, R₆, R₇ or R₈ is absent where W. X, Y, or Z, respectively, is —N═, O, S or absent;
R₁₅ is chosen from optionally substituted alkyl-, optionally substituted aryl-, optionally substituted aralkyl-, optionally substituted heteroaryl-, and optionally substituted heteroaralkyl-; and
R₁₇ is hydrogen, optionally substituted alkyl-, optionally substituted aryl-, optionally substituted aralkyl-, optionally substituted heteroaryl-, or optionally substituted hetero-aralkyl-, including single stereoisomers, mixtures of stereoisomers;
a pharmaceutically acceptable salt of a compound of Formula I;
a pharmaceutically acceptable solvate of a compound of Formula I; or
a pharmaceutically acceptable solvate of a pharmaceutically acceptable salt of a compound of Formula I.

2. A compound having the structure: 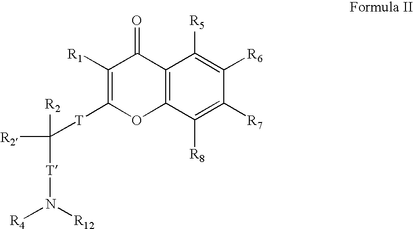
wherein:
T and T′ are independently a covalent bond or optionally substituted lower alkylene, provided that T and T′ are not both covalent bonds;
R₁ is chosen from hydrogen, optionally substituted alkyl-, optionally substituted aryl-, optionally substituted aralkyl-, optionally substituted heteroaryl-, and optionally substituted heteroaralkyl-;
R₂ and R_{2′} are independently chosen from hydrogen, optionally substituted alkyl-, optionally substituted aryl-, optionally substituted aralkyl-, optionally substituted heteroaryl-, and optionally substituted heteroaralkyl-; or R₂ and R₂ taken together form an optionally substituted 3- to 7-membered ring that optionally incorporates in the ring between zero and two heteroatoms selected from N, O, and S;
R₁₂ is chosen from hydrogen, optionally substituted alkyl-, optionally substituted aryl-, optionally substituted aralkyl-, optionally substituted heteroaryl-, optionally substituted heteroaralkyl-, —C(O)—R₃, and —S(O)₂—R₃ₐ;
R₄ is chosen from hydrogen, optionally substituted alkyl-, optionally substituted aryl-, optionally substituted aralkyl-, optionally substituted heteroaralkyl-, and optionally substituted heterocyclyl-;
or R₄ taken together with R₁₂ and the nitrogen to which they are bound, form an optionally substituted 5- to 12-membered nitrogen-containing heterocycle, which optionally incorporates from one to two additional heteroatoms, selected from N, O, and S, in the heterocycle ring;
or R₄ taken together with R₂ form an optionally substituted 5- to 12-membered nitrogen-containing heterocycle, which optionally incorporates from one to two additional heteroatoms, selected from N, O, and S in the heterocycle ring;
R₃ is chosen from hydrogen, optionally substituted alkyl-, optionally substituted aryl-, optionally substituted aralkyl-, optionally substituted heteroaryl-, optionally substituted heteroaralkyl-, R₁₅O— and R₁₇—NH—;
R₃ₐ is chosen from optionally substituted alkyl-, optionally substituted aryl-, optionally substituted aralkyl-, optionally substituted heteroaryl-, optionally substituted heteroaralkyl-, and R₁₇—NH—;
R₅, R₆, R₇ and R₈ are independently chosen from hydrogen, acyl, optionally substituted alkyl-, optionally substituted alkoxy, halogen, hydroxyl, nitro, cyano, optionally substituted amino, alkylsulfonyl-, alkylsulfonamido-, alkylthio-, carboxyalkyl-, carboxamido-, aminocarbonyl-, optionally substituted aryl and optionally substituted heteroaryl-, provided that R₅, R₆, R₇ or R₈ is absent where W, X, Y, or Z, respectively, is —N═, O, S or absent;
R₁₅ is chosen from optionally substituted alkyl-, optionally substituted aryl-, optionally substituted aralkyl-, optionally substituted heteroaryl-, and optionally substituted heteroaralkyl-; and
R₁₇ is hydrogen, optionally substituted alkyl-, optionally substituted aryl-, optionally substituted aralkyl-, optionally substituted heteroaryl-, or optionally substituted hetero-aralkyl-, including single stereoisomers, mixtures of stereoisomers;
a pharmaceutically acceptable salt of a compound of Formula II;
a pharmaceutically acceptable solvate of a compound of Formula II; or
a pharmaceutically acceptable solvate of a pharmaceutically acceptable salt of a compound of Formula II.

3. A compound having the structure: 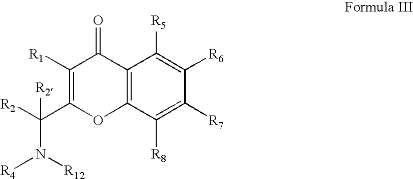
wherein:
R₁ is chosen from hydrogen, optionally substituted alkyl-, optionally substituted aryl-, optionally substituted aralkyl-, optionally substituted heteroaryl-, and optionally substituted heteroaralkyl-;
R₂ and R_{2′} are independently chosen from hydrogen, optionally substituted alkyl-, optionally substituted aryl-, optionally substituted aralkyl-, optionally substituted heteroaryl-, and optionally substituted heteroaralkyl-; or R₂ and R₂ taken together form an optionally substituted 3- to 7-membered ring which optionally incorporates from one to two additional heteroatoms, selected from N, O, and S, in the ring;
R₁₂ taken together with R₄, and the nitrogen to which they are bound, form an optionally substituted 5- to 12-membered nitrogen-containing heterocycle, which optionally incorporates from one to two additional heteroatoms, selected from N, O, and S, in the heterocycle ring, provided that such 5-membered nitrogen-containing heterocycle is not an optionally substituted imidazolyl or imidazolinyl ring; or
R₄ taken together with R₂, and the nitrogen to which R₄ is bound, form an optionally substituted 5- to 12-membered nitrogen-containing heterocycle, which optionally incorporates from one to two additional heteroatoms, selected from N, O, and S, in the heterocycle ring; and R₁₂ is chosen from hydrogen, optionally substituted alkyl-, optionally substituted aryl-, optionally substituted aralkyl-, optionally substituted heteroaralkyl-, and optionally substituted heterocyclyl-; and
R₅, R₆, R₇ and R₈ are independently chosen from hydrogen, acyl, optionally substituted alkyl-, optionally substituted alkoxy, halogen, hydroxyl, nitro, cyano, optionally substituted amino, alkylsulfonyl-, alkylsulfonamido-, alkylthio-, carboxyalkyl-, carboxamido-, aminocarbonyl-, optionally substituted aryl and optionally substituted heteroaryl-; including single stereoisomers, mixtures of stereoisomers;
a pharmaceutically acceptable salt of a compound of Formula III;
a pharmaceutically acceptable solvate of a compound of Formula III; or
a pharmaceutically acceptable solvate of a pharmaceutically acceptable salt of a compound of Formula III.

4. A compound according to claim 1, wherein one of T and T′ is optionally substituted alkylene, and the other is a covalent bond.

5. A compound according to claim 4, wherein one of T and T′ is optionally substituted methylene, and the other is a covalent bond.

6. A compound according to claim 1, wherein T and T′ are covalent bonds.

7. A compound according to claim 1, wherein one of W, X, Y, and Z is N, and the others are C.

8. A compound according to claim 1, wherein two of W, X, Y, and Z are N, and the others are C.

9. A compound according to claim 1, wherein the ring incorporating W, X, Y, and optionally Z is a pyridinyl, pyrimidinyl, pyridazinyl, pyrazinyl, imidazolyl, isoxazolyl, isothiazolyl, pyrazolyl, thiazolyl, oxazolyl, furanyl, pyrrolyl, or thiophenyl ring, each of which is optionally substituted.

10. A compound according to claim 1 any of claims 19, wherein only one of R₂ or R_{2′} is hydrogen.

11. A compound according to claim 1, wherein R₂ is optionally substituted C₁-C₄ alkyl and R₂ is hydrogen or optionally substituted C₁-C₄ alkyl.

12. A compound according to claim 11, wherein R_{2′} is hydrogen and R₂ is optionally substituted C₁-C₄ alkyl.

13. A compound according to claim 12, wherein R_{2′} is hydrogen and R₂ is ethyl or propyl.

14. A compound according to claim 13, wherein R₂ is i-propyl.

15. A compound according to claim 1, wherein R₁ is hydrogen, optionally substituted C₁-C₄ alkyl, optionally substituted phenyl-C₁-C₄-alkyl-, optionally substituted heteroaryl-C₁-C₄-alkyl-, optionally substituted naphthalenylmethyl, optionally substituted phenyl, or naphthyl.

16. A compound according to claim 15, wherein R₁ is optionally substituted phenyl-C₁-C₄-alkyl-, optionally substituted heteroaryl-C₁-C₄-alkyl-, optionally substituted naphthalenylmethyl-, optionally substituted phenyl, or napthyl.

17. A compound according to claim 16, wherein R₁ is naphthyl, phenyl, bromophenyl, chlorophenyl, methoxyphenyl, ethoxyphenyl, tolyl, dimethylphenyl, chorofluorophenyl, methylchlorophenyl, ethylphenyl, phenethyl, benzyl, chlorobenzyl, bromobenzyl, methylbenzyl, methoxybenzyl, cyanobenzyl, hydroxybenzyl, dichlorobenzyl, dimethoxybenzyl, or naphthalenylmethyl.

18. A compound according to claim 17, wherein R₁ is benzyl-, halobenzyl, methylbenzyl, hydroxybenzyl, cyanobenzyl-, methoxybenzyl-, or naphthalenylmethyl.

19. A compound according to claim 18, wherein R₁ is benzyl.

20. A compound according to claim 1, wherein R₄ taken together with R₂ form an optionally substituted 5- to 12-membered nitrogen-containing heterocycle, which optionally incorporates from one to two additional heteroatoms, selected from N, O, and S in the heterocycle ring.

21. A compound according to claim 1, wherein R₁₂ is chosen from optionally substituted C₁-C₁₃ alkyl; optionally substituted aralkyl; and optionally substituted heteroaralkyl.

22. A compound according to claim 21, wherein R₁₂ is benzyl or benzyl substituted with one or more of the following groups: carboxy, alkoxycarbonyl, cyano, halo, C₁-C₄ alkyl-, C₁-C₄ alkoxy, nitro, methylenedioxy, and trifluoromethyl.

23. A compound according to claim 1, wherein R₁₂ is —C(O)R₃ and R₃ is selected from optionally substituted C₁-C₈ alkyl-, optionally substituted aryl-C₁-C₄-alkyl-, optionally substituted heteroaryl-C₁-C₄-alkyl-, optionally substituted heteroaryl-, optionally substituted aryl-, R₁₅O—, and R₁₇NH—; R₁₅ is chosen from optionally substituted C₁-C₈-alkyl and optionally substituted aryl; and R₁₇ is chosen from hydrogen, optionally substituted C₁-C₈-alkyl, and optionally substituted aryl.

24. A compound according to claim 23, wherein R₃ is chosen from phenyl;
benzyl; phenoxymethyl-; halophenoxymethyl-; phenylvinyl-; heteroaryl-; heteroaryl-substituted with C₁-C₄ alkyl or C₁-C₄ alkyl substituted with halo; C₁-C₄ alkyl substituted with C₁-C₄ alkoxy-; benzyloxymethyl-; and phenyl substituted with one or more of the following substituents: halo, C₁-C₄ alkyl, C₁-C₄ alkyl substituted with hydroxy, C₁-C₄ alkoxy, C₁-C₄ alkyl substituted with C₁-C₄ alkoxy, nitro, formyl, carboxy, cyano, methylenedioxy, ethylenedioxy, acyl, —N-acyl, and trifluoromethyl.

25. A compound according to claim 24, wherein R₃ is chosen from phenyl, halophenyl, dihalophenyl, cyanophenyl, halo(trifluoromethyl)phenyl, hydroxymethlylphenyl, methoxymethylphenyl, methoxyphenyl, ethoxyphenyl, carboxyphenyl, formylphenyl, ethylphenyl, tolyl, methylenedioxyphenyl, ethylenedixoyphenyl, methoxychlorophenyl, dihydro-benzodioxinyl, methylhalophenyl, trifluoromethylphenyl, furanyl, C₁-C₄ alkyl substituted furanyl, trifluoromethylfuranyl, C₁-C₄ alkyl substituted trifluoromethylfuranyl, benzofuranyl, thiophenyl, C₁-C₄ alkyl substituted thiophenyl, benzothiophenyl, benzothiadiazolyl, pyridinyl, indolyl, methylpyridinyl, trifluoromethylpyridinyl, pyrrolyl, quinolinyl, picolinyl, pyrazolyl, C₁-C₄ alkyl substituted pyrazolyl, N-methylpyrazolyl, C₁-C₄ alkyl substituted N-methylpyrazolyl, C₁-C₄ alkyl substituted pyrazinyl, C₁-C₄ alkyl substituted isoxazolyl, benzoisoxazolyl, morpholinomethyl, methylthiomethyl, methoxymethyl, N-methyl imidazolyl, and imidazolyl.

26. A compound according to claim 25, wherein R₃ is tolyl, halophenyl, methylhalophenyl, hydroxymethylphenyl, halo(trifluoromethyl)phenyl-, methylenedioxyphenyl, formylphenyl or cyanophenyl.

27. A compound according to claim 23, wherein R₃ is R₁₇NH—; and R₁₇ is chosen from hydrogen, C₁-C₄ alkyl, cyclohexyl, phenyl, and phenyl substituted with halo, C₁-C₄ alkyl, trifluoromethyl, C₁-C₄ alkoxy, or C₁-C₄ alkylthio.

28. A compound according to claim 23, wherein R₃ is R₁₅O—; and R₁₅ is chosen from optionally substituted C₁-C₈ alkyl and optionally substituted aryl.

29. A compound according to claim 1 wherein R₁₂ is —SO₂R₃ₐ and R₃ₐ is chosen from C₁-C₁₃ alkyl; phenyl; naphthyl; phenyl substituted with halo, C₁-C₄ alkyl, C₁-C₄ alkoxy, cyano, nitro, methylenedioxy, or trifluoromethyl; biphenylyl; and heteroaryl.

30. A compound according to claim 1, wherein R₄ is chosen from hydrogen, optionally substituted C₁-C₁₃ alkyl, optionally substituted aryl, optionally substituted aryl-C₁-C₄-alkyl-, optionally substituted heterocyclyl, and optionally substituted heteroaryl-C₁-C₄-alkyl.

31. A compound according to claim 30, wherein R₄ is chosen from hydrogen; C₁-C₄alkyl; cyclohexyl; phenyl substituted with hydroxyl, C₁-C₄ alkoxy, or C₁-C₄ alkyl; benzyl; and R₁₆-alkylene-, wherein R₁₆ is hydroxyl, carboxy, (C₁-C₄ alkoxy)carbonyl; di(C₁-C₄ alkyl)amino-, (C₁-C₄ alkyl)amino-, amino, (C₁-C₄ alkoxy)carbonylamino-, C₁-C₄ alkoxy-, optionally substituted N-heterocyclyl-, or furanyl.

32. A compound according to claim 31, wherein R₄ is chosen from hydrogen, methyl, ethyl, propyl, butyl, cyclohexyl, carboxyethyl, carboxymethyl, methoxyethyl, hydroxyethyl, hydroxypropyl, dimethylaminoethyl, dimethylaminopropyl, diethylaminoethyl, diethylaminopropyl, aminopropyl, methylaminopropyl, 2,2-dimethyl-3-(dimethylamino)propyl, aminoethyl, aminobutyl, aminopentyl, aminohexyl, isopropylaminopropyl, diisopropylaminoethyl, 1-methyl-4-(diethylamino)butyl, (t-Boc)aminopropyl, hydroxyphenyl, benzyl, methoxyphenyl, methylmethoxyphenyl, dimethylphenyl, tolyl, ethylphenyl, (oxopyrrolidinyl)propyl, (methoxycarbonyl)ethyl, benzylpiperidinyl, pyridinylethyl, pyridinylmethyl, morpholinylethyl, morpholinylpropyl, piperidinyl, azetidinylmethyl, azetidinylethyl, azetidinylpropyl, pyrrolidinylethyl, pyrrolidinylpropyl, piperidinylmethyl, piperidinylethyl, imidazolylpropyl, imidazolylethyl, (ethylpyrrolidinyl)methyl, (methylpyrrolidinyl)ethyl, (methylpiperidinyl)propyl, (methylpiperazinyl)propyl, furanylmethyl, and indolylethyl.

33. A compound according to claim 31, wherein R₄ is R₁₆-alkylene-, wherein R₁₆ is amino, C₁-C₄ alkylamino-, di(C₁-C₄ alkyl)amino-, C₁-C₄ alkoxy-, hydroxyl, or N-heterocyclyl.

34. A compound according to claim 33, wherein R₄ is aminoethyl, aminopropyl, aminobutyl, aminopentyl, aminohexyl, methylaminoethyl, methylaminopropyl, methylaminobutyl, methylaminopentyl, methylaminohexyl, dimethylaminoethyl, dimethylaminopropyl, dimethylaminobutyl, dimethylaminopentyl, dimethylaminohexyl, ethylaminoethyl, ethylaminopropyl, ethylaminobutyl, ethylaminopentyl, ethylaminohexyl, diethylaminoethyl, diethylaminopropyl, diethylaminobutyyl, diethylaminopentyl, or diethylaminohexyl.

35. A compound according to claim 1, wherein R₁₂ and R₄ taken together with the nitrogen to which they are bound form an optionally substituted imidazolyl ring of the formula: 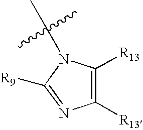
wherein
R₉ is chosen from hydrogen, optionally substituted C₁-C₈ alkyl, optionally substituted aryl, optionally substituted aryl-C₁-C₄-alkyl-, optionally substituted heteroaryl-C₁-C₄-alkyl-, optionally substituted aryl-C₁-C₄-alkoxy-, optionally substituted heteroaryl-C₁-C₄-alkoxy-, and optionally substituted heteroaryl-; and R₁₃ and R₁₃ are independently hydrogen, optionally substituted C₁-C₈ alkyl, optionally substituted aryl, or optionally substituted aryl-C₁-C₄-alkyl-.

36. A compound according to claim 1, wherein R₁₂ and R₄ taken together with the nitrogen to which they are bound form an optionally substituted imidazolinyl ring of the formula: 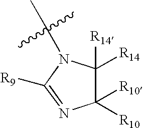
wherein R₉ is chosen from hydrogen, optionally substituted C₁-C₈ alkyl, optionally substituted aryl, optionally substituted aryl-C₁-C₄-alkyl-, and optionally substituted heteroaryl-; and R₁₀, R_{10′}, R₁₄, and R_{14′} are independently chosen from hydrogen, optionally substituted C₁-C₈ alkyl, optionally substituted aryl, and optionally substituted aryl-C₁-C₄-alkyl-.

37. A compound according to claim 1, wherein R₁₂ and R₄ form an optionally substituted diazepinone ring of the formula: 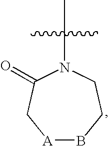
wherein
A and B are each independently chosen from C(R₂₀)(R₂₁), N(R₂₂), O, or S, wherein R₂₀ and R₂₁ are each independently selected from hydrogen, optionally substituted alkyl, optionally substituted aryl, and optionally substituted heteroaryl; and R₂₂ is hydrogen, optionally substituted alkyl, optionally substituted aralkyl, optionally substituted heteroaralkyl, optionally substituted alkylcarbonyl, optionally substituted arylcarbonyl, optionally substituted heteroarylcarbonyl, optionally substituted aralkylcarbonyl, optionally substituted heteroaralkylcarbonyl, optionally substituted alkoxycarbonyl, optionally substituted aryloxycarbonyl, optionally substituted heteroaryloxycarbonyl, optionally substituted aralkyloxycarbonyl, or optionally substituted heteroaralkyloxycarbonyl.

38. A compound according to claim 1, wherein R₁₂ and R₄ form an optionally substituted piperazine or diazepam of the formula: 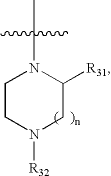
wherein
R₃, and R₃₂ are independently chosen from hydrogen, optionally substituted alkyl, optionally substituted aryl, optionally substituted heteroaryl, optionally substituted aralkyl, and optionally substituted heteroaralkyl; and n is 1 or 2.

39. A compound according to claim 1, wherein one of W, X, Y, and Z is N and the others are C; R₁ is benzyl, halobenzyl, methylbenzyl, hydroxybenzyl, cyanobenzyl, methoxybenzyl, or naphthalenylmethyl-; R₂ is optionally substituted C₁-C₄ alkyl; R₂, is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; T and T′ are independently a covalent bond or optionally substituted lower alkylene; and R₁₂ is —C(O)R₃, wherein R₃ is tolyl, halophenyl, methylhalophenyl-, hydroxymethylphenyl, halo(trifluoromethyl)phenyl-, methylenedioxyphenyl-, formylphenyl, or cyanophenyl; and R₄ is R₁₆-alkylene-, wherein R₁₆ is amino, C₁-C₄ alkylamino-, di(C₁-C₄ alkyl)amino-, C₁-C₄ alkoxy, hydroxyl, or N-heterocyclyl.

40. A compound according to claim 39, wherein R₂ is propyl.

41. A compound according to claim 1, wherein one of W, X, Y, and Z is N and the others are C; R₁ is benzyl, halobenzyl, methylbenzyl, hydroxybenzyl, cyanobenzyl, methoxybenzyl, or naphthalenylmethyl-; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; T and T′ are independently a covalent bond or optionally substituted lower alkylene; and R₄ taken together with R₁₂ form an optionally substituted imidazolinyl ring of the formula: 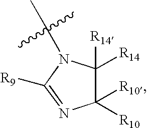
wherein R₁₀, R_{10′}, R₁₄, and R₁₄ are independently hydrogen or optionally substituted alkyl, and R₉ is optionally substituted phenyl.

42. A compound according to claim 41, wherein R₂ is propyl.

43. A compound according to claim 1, wherein one of W, X, Y, and Z is N and the others are C; R₁ is benzyl, halobenzyl, methylbenzyl, hydroxybenzyl, cyanobenzyl, methoxybenzyl, or naphthalenylmethyl-; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; T and T′ are independently a covalent bond or optionally substituted lower alkylene; and R₄ taken together with R₁₂ form an optionally substituted imidazolyl ring of the formula: 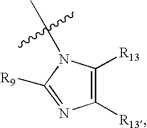
wherein R₁₃ is hydrogen; R_{13′} is hydrogen or optionally substutited alkyl; and R₉ is optionally substutited aryl.

44. A compound according to claim 43, wherein R₂ is propyl.

45. A compound according to claim 1, wherein one of W, X, Y, and Z is N and the others are C; R₁ is benzyl, halobenzyl, methylbenzyl, hydroxybenzyl, cyanobenzyl, methoxybenzyl, or naphthalenylmethyl-; R₂ is optionally substituted C₁-C₄ alkyl; R₂, is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; T and T′ are independently a covalent bond or optionally substituted lower alkylene; and R₄ taken together with R₁₂ form an optionally substituted imidazolidinyl ring.

46. A compound according to claim 45, wherein R₂ is propyl.

47. A compound according to claim 1, wherein one of W, X, Y, and Z is N and the others are C; R₁ is benzyl, halobenzyl, methylbenzyl, hydroxybenzyl, cyanobenzyl, methoxybenzyl, or naphthalenylmethyl-; R₂ is optionally substituted C₁-C₄ alkyl; R₂ is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; T and T′ are independently a covalent bond or optionally substituted lower alkylene; and R₄ taken together with R₁₂ form an optionally substituted piperazinyl ring.

48. A compound according to claim 47, wherein R₂ is propyl.

49. A compound according to claim 1, wherein one of W, X, Y, and Z is N and the others are C; R₁ is benzyl, halobenzyl, methylbenzyl, hydroxybenzyl, cyanobenzyl, methoxybenzyl, or naphthalenylmethyl-; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; T and T′ are independently a covalent bond or optionally substituted lower alkylene; and R₄ taken together with R₁₂ form an optionally substituted diazepinoyl ring.

50. A compound according to claim 49, wherein R₂ is propyl.

51. A compound according to claim 2, wherein R₁ is benzyl, halobenzyl, methylbenzyl, hydroxybenzyl, methoxybenzyl-, cyanobenzyl, or naphthalenylmethyl-; R₂ is optionally substituted C₁-C₄ alkyl; R₂ is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; one of T and T′ is a covalent bond and the other is optionally substituted lower alkylene; and R₁₂ is —C(O)R₃, wherein R₃ is tolyl, halophenyl, methylhalophenyl-, hydroxymethylphenyl, halo(trifluoromethyl)phenyl-, methylenedioxyphenyl-, formylphenyl, or cyanophenyl; and R₄ is R₁₆-alkylene-, wherein R₁₆ is amino, C₁-C₄ alkylamino-, di(C₁-C₄ alkyl)amino-, C₁-C₄ alkoxy, hydroxyl, or N-heterocyclyl.

52. A compound according to claim 51, wherein R₂ is propyl.

53. A compound according to claim 2, wherein R₁ is benzyl, halobenzyl, methylbenzyl, hydroxybenzyl, methoxybenzyl-, cyanobenzyl, or naphthalenylmethyl-; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; one of T and T′ is a covalent bond and the other is optionally substituted lower alkylene; and R₄ taken together with R₁₂ form an optionally substituted imidazolinyl ring of the formula: 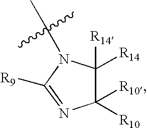
wherein R₁₀, R_{10′}, R₁₄, and R_{14′} are independently hydrogen or optionally substituted alkyl, and R₉ is optionally substituted phenyl.

54. A compound according to claim 53, wherein R₂ is propyl.

55. A compound according to claim 2, wherein R₁ is benzyl, halobenzyl, methylbenzyl, hydroxybenzyl, methoxybenzyl-, cyanobenzyl, or naphthalenylmethyl-; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; one of T and T′ is a covalent bond and the other is optionally substituted lower alkylene; and R₄ taken together with R₁₂ form an optionally substituted imidazolyl ring of the formula: 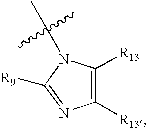
wherein R₁₃ is hydrogen; R_{13′} is hydrogen or optionally substutited alkyl; and R₉ is optionally substutited aryl.

56. A compound according to claim 55, wherein R₂ is propyl.

57. A compound according to claim 2, wherein R₁ is benzyl, halobenzyl, methylbenzyl, hydroxybenzyl, methoxybenzyl-, cyanobenzyl, or naphthalenylmethyl-; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; one of T and T′ is a covalent bond and the other is optionally substituted lower alkylene; and R₄ taken together with R₁₂ form an optionally substituted imidazolidinyl ring.

58. A compound according to claim 57, wherein R₂ is propyl.

59. A compound according to claim 2, wherein R₁ is benzyl, halobenzyl, methylbenzyl, hydroxybenzyl, methoxybenzyl-, cyanobenzyl, or naphthalenylmethyl-; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; one of T and T′ is a covalent bond and the other is optionally substituted lower alkylene; and R₄ taken together with R₁₂ form an optionally substituted piperazinyl ring.

60. A compound according to claim 59, wherein R₂ is propyl.

61. A compound according to claim 2, wherein R₁ is benzyl, halobenzyl, methylbenzyl, hydroxybenzyl, methoxybenzyl-, cyanobenzyl, or naphthalenylmethyl-; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; one of T and T′ is a covalent bond and the other is optionally substituted lower alkylene; and R₄ taken together with R₁₂ form an optionally substituted diazepinoyl ring.

62. A compound according to claim 61, wherein R₂ is propyl.

63. A compound according to claim 3, wherein R₁ is benzyl, halobenzyl, methylbenzyl, hydroxybenzyl, methoxybenzyl, cyanobenzyl, or naphthalenylmethyl; R₂ is optionally substituted C₁-C₄ alkyl; R₂ is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; and R₄ taken together with R₁₂ form an optionally substituted piperazinyl ring.

64. A compound according to claim 63, wherein R₂ is propyl.

65. A compound according to claim 3, wherein R₁ is benzyl, halobenzyl, methylbenzyl, hydroxybenzyl, methoxybenzyl, cyanobenzyl, or naphthalenylmethyl; R₂ is optionally substituted C₁-C₄ alkyl; R_{2′} is hydrogen; R₅, R₆, R₇, and R₈ are independently chosen from hydrogen, amino, alkylamino, dialkylamino, hydroxyl, halogen, C₁-C₄ alkyl, C₁-C₄ haloalkyl, C₁-C₄ alkoxy, C₁-C₄ haloalkoxy, and cyano; and R₄ taken together with R₁₂ form an optionally substituted diazepinoyl ring.

66. A compound according to claim 65, wherein R₂ is propyl.

67. A compound selected from
N-(3-aminopropyl)-N-{1-[7-chloro-4-oxo-3-(phenylmethyl)-4H-pyrano[2,3-b]pyridin-2-yl]-2-methylpropyl}-4-methylbenzamide;
2-{1-[2-(1,3-benzodioxol-5-yl)-4,5-dihydro-1H-imidazol-1-yl]-2-methylpropyl}-7-chloro-3-{[3-(methyloxy)phenyl]methyl}-4H-pyrano[2,3-b]pyridin-4-one;
4-{1-[7-Chloro-4-oxo-3-(phenylmethyl)-4H-chromen-2-yl]-2-methylpropyl} hexahydro-5H-1,4-diazepin-5-one; and
7-Chloro-2-[2-methyl-1-(7-oxohexahydro-1H-1,4-diazepin-1-yl)propyl]-3-(phenylmethyl)-4H-pyrano[2,3-b]pyridin-4-one.

68. A compound according to claim 1, wherein R₂ and R_{2′} are each attached to a stereogenic center having an R-configuration, or a pharmaceutically acceptable salt or solvate thereof.

69. A composition comprising a pharmaceutical excipient and a compound, salt, or solvate thereof of claim 1.

70. A composition according to claim 69, wherein said composition further comprises a chemotherapeutic agent other than a compound of Formula I, II, or III, or a pharmaceutical salt or solvate thereof.

71. A composition according to claim 70, wherein said composition further comprises a taxane.

72. A composition according to claim 70, wherein said composition further comprises a vinca alkaloid.

73. A composition according to claim 70, wherein said composition further comprises a topoisomerase I inhibitor.

74. A method of inhibiting KSP which comprises contacting said kinesin with an effective amount of a compound according to claim 1.

75. A method for the treatment of a cellular proliferative disease comprising administering to a subject in need thereof a compound according to claim 1.

76. A method according to claim 75 wherein said disease is selected from the group consisting of cancer, hyperplasias, restenosis, cardiac hypertrophy, immune disorders, and inflammation.

77. (canceled)

78. (canceled)
